(19) United States
(12) Patent Application Publication
(10) Pub. No.: US 2024/0269329 A1
(43) Pub. Date: Aug. 15, 2024

(54) GENE THERAPY FOR DUCHENNE MUSCULAR DYSTROPHY

(71) Applicant: University of Florida Research Foundation, Incorporated, Gainesville, FL (US)

(72) Inventor: Hugh Lee Sweeney, Gainesville, FL (US)

(73) Assignee: University of Florida Research Foundation, Incorporated, Gainesville, FL (US)

(21) Appl. No.: 18/567,763

(22) PCT Filed: Jun. 7, 2022
(86) PCT No.: PCT/US2022/032570

§ 371 Date: Dec. 6, 2023

Related U.S. Application Data
(60) Provisional application No. 63/197,976, filed on Jun. 7, 2021.

(51) Int. Cl. A61K 48/00; A61K 31/4985; A61K 31/519; A61K 38/00; C07K 14/47; C12N 15/86

(52) U.S. Cl. CPC A61K 48/0058; A61K 31/4985; A61K 31/519; A61K 48/0075; C07K 14/4708; C12N 15/86; A61K 38/00; C07K 2319/73; C12N 2750/14143

(57) ABSTRACT

The disclosure provides compositions and methods for producing micro-dystrophin proteins, and the use thereof (e.g., in gene therapy). The disclosure also provides skeletal muscle-specific promoters and the use thereof (e.g., in gene therapy). The disclosure further provides catheter-based methods of delivering gene therapy vectors to skeletal muscle and/or cardiac muscle.

### RELATED APPLICATIONS

This application claims the benefit under 35 U.S.C. 119(e) of the filing date of U.S. Provisional Application Ser. No. 63/197,976, filed Jun. 7, 2021, entitled “GENE THERAPY FOR DUCHENNE MUSCULAR DYSTROPHY”, the entire contents of which are incorporated herein by reference.

### BACKGROUND

Duchenne muscular dystrophy (DMD) is a genetic disorder characterized by progressive muscle degeneration and weakness. DMD is caused by mutations in the dystrophin gene. The multifaceted pathologies associated with DMD represent major challenges to the development and clinical implementation of effective therapeutics for the disease. Adeno-associated virus (AAV) gene therapy, delivering a micro-dystrophin construct to all striated muscles, is now poised to bring about transformative alterations in DMD disease course.

However, a number of significant problems remain. Accordingly, there is a need for alternative gene therapies for treating DMD.

### SUMMARY

In one aspect, the present disclosure provides a recombinant nucleic acid comprising a nucleotide sequence encoding a micro-dystrophin protein comprising: an amino-terminal actin-binding domain, hinge domain 1, spectrin-like repeat 1, and spectrin-like repeat 17, and wherein the micro-dystrophin protein does not contain spectrin-like repeat 2 and/or spectrin-like repeat 3 of dystrophin. In some embodiments, the micro-dystrophin protein further comprises spectrin-like repeat 18 and/or spectrin-like repeat 19.

In one aspect, the present disclosure provides a recombinant nucleic acid comprising a nucleotide sequence encoding a micro-dystrophin protein comprising: an amino-terminal actin-binding domain, hinge domain 1, spectrin-like repeat 1, spectrin-like repeat 17, spectrin-like repeat 18, and spectrin-like repeat 19, and wherein the micro-dystrophin protein does not contain spectrin-like repeat 2 and/or spectrin-like repeat 3 of dystrophin.

In some embodiments, the micro-dystrophin protein further comprises one or more of: spectrin-like repeat 16, hinge domain 3, spectrin-like repeat 21, spectrin-like repeat 22, spectrin-like repeat 24, hinge domain 4, and dystroglycan binding site of dystrophin. In some embodiments, the micro-dystrophin protein further comprises one or more syntrophin binding domains of dystrophin. In some embodiments, the micro-dystrophin protein further comprises one or more coiled coil domains of dystrophin.

In some embodiments, the spectrin-like repeat 1 is directly coupled to the spectrin-like repeat 17.

In some embodiments, the spectrin-like repeat 19 is directly coupled to spectrin-like repeat 24. In some embodiments, the spectrin-like repeat 19 is directly coupled to hinge domain 4.

In some embodiments, the spectrin-like repeat 17 is directly coupled to hinge domain 3 or spectrin-like repeat 22.

In one aspect, the present disclosure provides a recombinant nucleic acid comprising a nucleotide sequence encoding a micro-dystrophin protein comprising: an amino-terminal actin-binding domain, hinge domain 1, spectrin-like repeat 1, spectrin-like repeat 2, and spectrin-like repeat 17, and wherein the micro-dystrophin protein does not contain spectrin-like repeat 3 of dystrophin. In some embodiments, the micro-dystrophin protein further comprises spectrin-like repeat 16.

In one aspect, the present disclosure provides a recombinant nucleic acid comprising a nucleotide sequence encoding a micro-dystrophin protein comprising: an amino-terminal actin-binding domain, hinge domain 1, spectrin-like repeat 1, spectrin-like repeat 2, spectrin-like repeat 16, and spectrin-like repeat 17, and wherein the micro-dystrophin protein does not contain spectrin-like repeat 3 of dystrophin.

In some embodiments, the micro-dystrophin protein further comprises one or more of: hinge domain 3, spectrin-like repeat 22, spectrin-like repeat 23, spectrin-like repeat 24, hinge domain 4, and dystroglycan binding site of dystrophin. In some embodiments, the micro-dystrophin protein further comprises one or more syntrophin binding domains of dystrophin. In some embodiments, the micro-dystrophin protein further comprises one or more coiled coil domains of dystrophin.

In some embodiments, the spectrin-like repeat 2 is directly coupled to spectrin-like repeat 16. In some embodiments, the spectrin-like repeat 2 is directly coupled to spectrin-like repeat 17.

In some embodiments, the spectrin-like repeat 17 is directly coupled to hinge domain 3.

In some embodiments, the spectrin-like repeat 17 is directly coupled to spectrin-like repeat 24. In some embodiments, the spectrin-like repeat 17 is directly coupled to hinge domain 4.

In some embodiments, the micro-dystrophin protein comprises the region of dystrophin from spectrin-like repeat 24 to the C-terminus. In some embodiments, the micro-dystrophin protein comprises the region of dystrophin from spectrin-like repeat 24 to the end of the proline rich region following the first coiled coil domain. In some embodiments, the micro-dystrophin protein comprises the region of dystrophin from hinge domain 4 to the C-terminus.

In some embodiments, the micro-dystrophin protein comprises the region of dystrophin from hinge domain 4 to the end of the proline rich region following the first coiled coil domain.

In some embodiments, the micro-dystrophin protein comprises the region of dystrophin from hinge domain 4 to the end of the second syntrophin binding domain.

In some embodiments, the recombinant nucleic acid is less than 5 kb in length.

In some embodiments, a promoter is operably linked to the nucleotide sequence encoding the micro-dystrophin protein. In some embodiments, the promoter is a cardiac-specific promoter.

In some embodiments, the cardiac-specific promoter is a cardiac troponin T (cTnT) promoter. In some embodiments, the promoter is a skeletal muscle-specific promoter. In some embodiments, the skeletal muscle-specific promoter is a skeletal muscle alpha-actin promoter. In some embodiments, the skeletal-muscle specific promoter comprises a nucleotide sequence that is at least 80% identical to SEQ ID NO: 78 or SEQ ID NO: 80.

In some embodiments, the present disclosure provides a recombinant nucleic acid comprising a nucleotide sequence encoding a micro-dystrophin protein, wherein the nucleotide sequence encoding the micro-dystrophin protein comprises a nucleotide sequence that is at least 80% identical to the nucleotide sequence of any one of SEQ ID Nos. 7-12, 22-39, 92-97, 109-114, and 116-118.

In some embodiments, the present disclosure provides a recombinant nucleic acid comprising a nucleotide sequence encoding a micro-dystrophin protein that comprises an amino acid sequence that is at least 80% identical to the amino acid sequence of any one of SEQ ID Nos. 1-6, 13-21, 86-91, 98, 106-108, and 115.

In some embodiments, the present disclosure provides a micro-dystrophin protein encoded by a recombinant nucleic acid of the disclosure.

In some embodiments, the present disclosure provides a micro-dystrophin protein comprising an amino acid sequence that is at least 80% identical to the amino acid sequence of any one of SEQ ID Nos. 1-6, 13-21, 86-91, 98, 106-108, and 115.

In one aspect, the present disclosure provides a skeletal muscle-specific promoter. In one aspect, the present disclosure provides an isolated nucleic acid comprising a nucleotide sequence that is at least 80% identical to the nucleotide sequence of SEQ ID NO: 78 or SEQ ID NO: 80.

In some embodiments, the nucleotide sequence that is at least 80% identical to the nucleotide sequence of SEQ ID NO: 78 or SEQ ID NO: 80 is operably linked to a nucleotide sequence that is at least 80% identical to the nucleotide sequence of any one of SEQ ID Nos. 7-12, 22-39, 92-97, 109-114, and 116-118.

In some embodiments, the nucleotide sequence that is at least 80% identical to the nucleotide sequence of SEQ ID NO: 78 or SEQ ID NO: 80 is operably linked to a nucleotide sequence that encodes a micro-dystrophin protein that comprises an amino acid sequence that is at least 80% identical to the amino acid sequence of any one of SEQ ID Nos. 1-6,13-21, 86-91, 98, 106-108, and 115.

In one aspect, the present disclosure provides a recombinant adeno-associated virus (rAAV) vector comprising a recombinant nucleic acid or isolated nucleic acid of the disclosure.

In one aspect, the present disclosure provides an rAAV particle comprising a rAAV vector of the disclosure encapsidated in an AAV capsid. In some embodiments, the AAV capsid comprises a capsid protein derived from AAV1, AAV2, AAV3, AAV6, AAV7, AAV8, AAVrh.74, AAVrh.10, AAV2/6 or AAV9 serotypes.

In one aspect, the present disclosure provides a composition comprising an rAAV particle.

In one aspect, the present disclosure provides a method of treating a skeletal muscle disorder in a subject in need thereof, comprising administering to the subject a recombinant or isolated nucleic acid of the disclosure, a micro-dystrophin protein of the disclosure, an rAAV particle of the disclosure, or a composition of the disclosure.

In one aspect, the present disclosure provides a method of treating Duchenne muscular dystrophy (DMD) in a subject in need thereof, comprising administering to the subject a recombinant or isolated nucleic acid of the disclosure, a micro-dystrophin protein of the disclosure, an rAAV particle of the disclosure, or a composition of the disclosure.

In one aspect, the present disclosure provides a method of treating Duchenne muscular dystrophy (DMD) in a subject in need thereof, the method comprising delivering to the subject a first recombinant adeno-associated virus (rAAV) particle comprising a nucleotide sequence encoding a first micro-dystrophin protein to cardiac muscle and a second rAAV particle comprising a nucleotide sequence encoding a second micro-dystrophin to skeletal muscle.

In some embodiments, the first rAAV particle comprises a cardiac-specific promoter operably linked to the nucleotide sequence encoding the first micro-dystrophin protein. In some embodiments, the second rAAV particle comprises a skeletal muscle-specific promoter operably linked to the nucleotide sequence encoding the second micro-dystrophin protein.

In some embodiments, the first and second rAAV particles are of the same serotype. In some embodiments, the first and second rAAV particles are of different serotypes. In some embodiments, the first rAAV particle comprises a capsid protein derived from AAV9, AAVrh.74, or AAVrh.10. In some embodiments, the second rAAV particle comprises a capsid protein derived from AAV8.

In some embodiments, the first and second rAAV particles are delivered by the same method. In some embodiments, the first and second rAAV particles are delivered by different methods.

In some embodiments, the first and second rAAV particles are delivered via one or more catheters. In some embodiments, the method comprises: (i) introducing a catheter into the femoral artery and advancing to the heart; (ii) delivering the first rAAV particle into the left and right coronary arteries; (iii) retracting the catheter to the aortic arch; and (iv) delivering the second rAAV particle to the subclavian and/or carotid arteries. In some embodiments, the method further comprises (v) retracting the catheter into the descending aorta and delivering the second rAAV particle to skeletal muscle via descending aortic branches.

In some embodiments, the method further comprises administering a vasodilator to the subject prior to the delivery of the first and second rAAV particles.

In some embodiments, the vasodilator is a PDE5 inhibitor. In some embodiments, the PDE5 inhibitor is sildenafil or tadalafil.

In some embodiments, the vasodilator is administered 10 minutes, 20 minutes, 30 minutes, 40 minutes, 50 minutes, or 1 hour prior to the delivery of first rAAV and/or second rAAV.

In some embodiments, a blood pressure cuff is inflated on each limb at the time of delivery of the first rAAV particle and/or the second rAAV particle.

In some embodiments, the skeletal muscle is fast-twitch or slow-twitch.

In some embodiments, the first micro-dystrophin protein comprises: an amino-terminal actin-binding domain, hinge domain 1, spectrin-like repeat 1, and spectrin-like repeat 17, and wherein the micro-dystrophin protein does not contain spectrin-like repeat 2 and/or spectrin-like repeat 3 of dystrophin. In some embodiments, the first micro-dystrophin protein comprises: an amino-terminal actin-binding domain, hinge domain 1, spectrin-like repeat 1, spectrin-like repeat 17, spectrin-like repeat 18, and spectrin-like repeat 19, and wherein the micro-dystrophin protein does not contain spectrin-like repeat 2 and/or spectrin-like repeat 3 of dystrophin. In some embodiments, the first micro-dystrophin protein further comprises one or more of: spectrin-like repeat 16, hinge domain 3, spectrin-like repeat 21, spectrin-like repeat 22, spectrin-like repeat 24, hinge domain 4, and dystroglycan binding site of dystrophin. In some embodiments, the first micro-dystrophin protein further comprises one or more syntrophin binding domains of dystrophin. In some embodiments, the first micro-dystrophin protein further comprises one or more coiled coil domains of dystrophin. In some embodiments, the spectrin-like repeat 1 of the first micro-dystrophin protein is directly coupled to the spectrin-like repeat 17. In some embodiments, the spectrin-like repeat 19 of the first micro-dystrophin protein is directly coupled to spectrin-like repeat 24. In some embodiments, the spectrin-like repeat 19 of the first micro-dystrophin protein is directly coupled to hinge domain 4. In some embodiments, the spectrin-like repeat 17 is directly coupled to hinge domain 3. In some embodiments, the spectrin-like repeat 17 is directly coupled to spectrin-like repeat 22.

In some embodiments, the second micro-dystrophin protein comprises: an amino-terminal actin-binding domain, hinge domain 1, spectrin-like repeat 1, spectrin-like repeat 2, and spectrin-like repeat 17, and wherein the micro-dystrophin protein does not contain spectrin-like repeat 3 of dystrophin. In some embodiments, the second micro-dystrophin protein comprises: an amino-terminal actin-binding domain, hinge domain 1, spectrin-like repeat 1, spectrin-like repeat 2, spectrin-like repeat 16, and spectrin-like repeat 17, and wherein the micro-dystrophin protein does not contain spectrin-like repeat 3 of dystrophin. In some embodiments, the second micro-dystrophin protein further comprises one or more of: hinge domain 3, spectrin-like repeat 22, spectrin-like repeat 23, spectrin-like repeat 24, hinge domain 4, and dystroglycan binding site of dystrophin. In some embodiments, the second micro-dystrophin protein further comprises one or more syntrophin binding domains of dystrophin. In some embodiments, the second micro-dystrophin protein further comprises one or more coiled coil domains of dystrophin. In some embodiments, the spectrin-like repeat 2 of the second micro-dystrophin protein is directly coupled to spectrin-like repeat 16. In some embodiments, the spectrin-like repeat 2 of the second micro-dystrophin protein is directly coupled to spectrin-like repeat 17. In some embodiments, the spectrin-like repeat 17 of the second micro-dystrophin protein is directly coupled to hinge domain 3. In some embodiments, the spectrin-like repeat 17 of the second micro-dystrophin protein is directly coupled to spectrin-like repeat 24. In some embodiments, the spectrin-like repeat 17 of the second micro-dystrophin protein is directly coupled to hinge domain 4.

In some embodiments, the first or second micro-dystrophin protein comprises the region of dystrophin from spectrin-like repeat 24 to the C-terminus. In some embodiments, the first or second micro-dystrophin protein comprises the region of dystrophin from spectrin-like repeat 24 to the end of the proline rich region following the first coiled coil domain. In some embodiments, the first or second micro-dystrophin protein comprises the region of dystrophin from hinge domain 4 to the C-terminus. In some embodiments, the first or second micro-dystrophin protein comprises the region of dystrophin from hinge domain 4 to the end of the proline rich region following the first coiled coil domain. In some embodiments, the first or second micro-dystrophin protein comprises the region of dystrophin from hinge domain 4 to the end of the second syntrophin binding domain.

In one aspect, the present disclosure provides a method of delivering an rAAV particle to a skeletal muscle in a subject, the method comprising delivering the rAAV particle via a catheter.

In some embodiments, the method comprises delivering the rAAV particle to the subclavian and/or carotid arteries. In some embodiments, the catheter is first introduced into the femoral artery and advanced to the subclavian and/or carotid arteries. In some embodiments, the method further comprises retracting the catheter into the descending aorta and delivering the rAAV particle to skeletal muscle via descending aortic branches.

In some embodiments, the method comprises administering a vasodilator. In some embodiments, the vasodilator is a PDE5 inhibitor. In some embodiments, the PDE5 inhibitor is sildenafil or tadalafil. In some embodiments, the vasodilator is administered 10 minutes, 20 minutes, 30 minutes, 40 minutes, 50 minutes, or 1 hour prior to the delivery of the rAAV particle.

In some embodiments, the method further comprises inflating a blood pressure cuff on each limb at the time of delivery of the rAAV particle.

In some embodiments, the skeletal muscle is fast-twitch or slow-twitch.

### BRIEF DESCRIPTION OF DRAWINGS

FIG. 1 shows a diagram of dystrophin and its interactions, showing known syntrophin binding sites.

FIGS. 2A and 2B show a diagram of full-length dystrophin showing order of Repeats (R) and hinges (H), as well as the cysteine-rich portion of the C-terminus (CR), which is commonly included in micro-dystrophins and referred to as the truncated C-terminus, which is then followed by the rest of the C-terminus (CT).

FIGS. 3A and 3B show skeletal muscle function at one month following injection of AAV micro-dystrophins. FIG. 3A shows the force per unit area values (specific tension) for the diaphragm and EDL muscles that received delivery of either one of three micro-dystrophins in clinical trials (PF-06939926, SRP-9001-10 or SGT-001) or one of the exemplary micro-dystrophins of the disclosure (Nterm-R1_R17_H3_R21-R22_H4 to end of syntrophin region, Nterm-R2_R17_H3_R22_H4 to end of syntrophin region, Nterm-R1_R17_R22_H4 to 1st coiled coil, or Nterm-R1_R16-R17_R21-R22_H4 to end of syntrophin region). “H4 to end of syntrophin region” is shown as “H4-SynBS.” FIG. 3B shows the ability of each of these micro-dystrophins to protect the skeletal muscles from contraction-induced injury.

FIG. 4 shows cardiac function at one year of age following injection of AAV micro-dystrophins at 5 weeks of age. Shown in the two graphs are cardiac output values for separate groups of mice that received the indicated AAV-micro-dystrophins. Cardiac outputs (mis/minute) were determined by measuring stroke volume using a Vevo3100 echocardiographic instrument and multiplying by heart rate.

FIG. 5 shows ventricular end diastolic volumes (EDV) at one year of age following injection of AAV micro-dystrophins at 5 weeks of age. Shown in the two graphs are cardiac EDV values (determined using a Vevo3100 echocardiographic instrument) for separate groups of mice that received the indicated AAV-micro-dystrophins.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure.

### Overview

The present disclosure relates to compositions and methods of cardiac muscle and/or skeletal muscle gene therapy, e.g., for treating DMD.

Gene therapy for DMD has moved into the clinic, with four ongoing clinical trials and more planned. The ongoing trials deliver a truncated version of the deficient protein, dystrophin, to heart and skeletal muscle using AAV. The reason for delivery of a truncated protein, known as micro-dystrophin is that the full length protein will not fit within the packaging limits of AAV.

However, a number of significant problems remain. There is a need to prevent and resolve fibrosis in order to improve the efficiency of delivery and enable the use of lower viral doses. Additional therapeutics may be needed to slow the skeletal muscle disease progression that remains after micro-dystrophin therapy. Perhaps the most critical issue facing AAV gene therapy for DMD is that current micro-dystrophin designs and vector doses are optimized for skeletal muscle without sufficient evaluation of the potential impact on cardiac muscle function.

The micro-dystrophins in trials have resulted from more than a decade of investigation of constructs in the dystrophin-deficient mdx mouse. The inventors have recently analyzed these constructs in clinical trials in a newer, more severe mouse model (D2.mdx mouse) that is much more indicative of the human disease. Without wishing to be bound by theory, based on the results of these mouse studies, it is predicted that the long-term consequences of existing gene therapies will be a form of DMD with severe cardiomyopathy and slowly failing skeletal muscle function. Accordingly, the inventors have identified the need for a different approach to micro-dystrophin delivery to skeletal muscle versus to the heart.

Designing different micro-dystrophin constructs that are optimal for each tissue will significantly improve the therapeutic effects of gene therapy with micro-dystrophin. In some embodiments, this involves simultaneous delivery of two separate vectors, one delivered systemically to skeletal muscles and one delivered directly into the coronary arteries of the heart, using different micro-dystrophin constructs and doses. In some embodiments, a small, skeletal muscle (fast and slow)-specific promoter is used for skeletal muscle. In some embodiments, a cardiac-specific promoter is used for the heart. The disclosure provides micro-dystrophin constructs with improved cardiac-corrective properties, likely to be safer and more beneficial than those in current trials. By combining additional gene therapy approaches with micro-dystrophin, long-lasting benefits to both striated muscle types can be achieved.

Accordingly, in one aspect, the present disclosure provides compositions and methods for producing micro-dystrophin proteins, and the use thereof. The present disclosure provides nucleic acids encoding micro-dystrophin proteins optimized for either skeletal muscle or cardiac muscle. In some embodiments, the nucleic acids encoding micro-dystrophin proteins are optimized for cardiac muscle. In some embodiments, the nucleic acids encoding micro-dystrophin proteins are optimized for skeletal muscle.

In another aspect, the present disclosure relates to skeletal muscle-specific promoters and the use thereof.

In yet another aspect, the present disclosure relates to localized delivery (e.g., catheter-based delivery) of gene therapy vectors (e.g., rAAV vectors) to skeletal muscle and/or cardiac muscle. The inventors have found that catheter-based delivery of gene therapy vectors to skeletal muscle and/or cardiac muscle is more efficient and allows for a better distribution of the gene therapy vectors, enabling the use of lower doses of the gene therapy vectors. This in turn mitigates any unfavorable immune responses to the gene therapy vectors. In some embodiments, the present disclosure provides a catheter-based method of delivering a gene therapy vector (e.g., an rAAV vector) to skeletal muscle. In some embodiments, the present disclosure provides a catheter-based method of simultaneously delivering one or more gene therapy vectors (e.g., one or more rAAV vectors) to skeletal muscle and cardiac muscle. In some embodiments, the gene therapy vector targeted to skeletal muscle comprises a nucleic acid encoding a micro-dystrophin protein that is optimized for skeletal muscle and the gene therapy vector targeted to cardiac muscle comprises a nucleic acid encoding a micro-dystrophin protein that is optimized for cardiac muscle.

### Micro-Dystrophins

A micro-dystrophin gene as used herein refers to a truncated dystrophin gene that is generally less than 5 kb in length. A micro-dystrophin gene is generally small enough to fit into an AAV vector. In some embodiments, a micro-dystrophin gene is less than about 4900 bp in length. In some embodiments, a micro-dystrophin gene is less than about 4800 bp in length. In some embodiments, a micro-dystrophin gene is less than about 4700, 4600, or 4500 bp in length.

In some embodiments, a micro-dystrophin gene is between about 3600-5000 bp in length. In some embodiments, a micro-dystrophin gene is between about 3600-4800 bp in length, about 3600-4500 bp in length, about 3300-5000 bp in length, about 3300 to 4800 bp in length, about 3300 to 4500 bp in length, about 3000-5000 bp in length, about 3000-4800 bp in length, or about 3000-4500 bp in length. A micro-dystrophin protein as used herein is less than about 1700 aa in length. In some embodiments, a micro-dystrophin protein is less than about 1666 aa in length. In some embodiments, a micro-dystrophin protein is less than about 1600 aa in length. In some embodiments, a micro-dystrophin protein is between about 1200-1700 aa in length. In some embodiments, a micro-dystrophin protein is between about 1200-1666 aa in length, about 1200-1600 aa in length, about 1100-1700 aa in length, about 1100-1666 aa in length, about 1100 to 1600 aa in length, about 1100-1700 aa in length, about 1000-1666 aa in length, or about 1000-1600 aa in length. A micro-dystrophin gene may include naturally occurring dystrophin sequences from any species and variants derived from such genes by mutagenesis, or other modifications.

### Cardiac Muscle Micro-Dystrophins

The present disclosure provides micro-dystrophin genes and proteins optimized for cardiac muscle. The inventors have found that certain combinations of domains from full-length dystrophin are beneficial for cardiac muscle.

In some embodiments, a micro-dystrophin protein optimized for cardiac muscle does not comprise spectrin-like repeat 2 and/or spectrin-like repeat 3 of dystrophin. In some embodiments, a micro-dystrophin protein does not comprise spectrin-like repeat 2. In some embodiments, a micro-dystrophin protein does not comprise spectrin-like repeat 3. In some embodiments, a micro-dystrophin protein does not comprise spectrin-like repeat 2 and spectrin-like repeat 3. The inventors have found that including spectrin-like repeat 1 but not spectrin-like repeat 2 and/or spectrin-like repeat 3 is beneficial to the heart as such micro-dystrophin proteins outcompete utrophin for its membrane binding in the heart to a lesser extent, thus mitigating the harmful effects of displacing utrophin. Harmful effects of displacing utrophin include cardiomyopathy and heart failure.

In some embodiments, a micro-dystrophin protein optimized for cardiac muscle comprises an amino-terminal actin-binding domain, hinge domain 1, spectrin-like repeat 1, and spectrin-like repeat 17. In some embodiments, the micro-dystrophin protein further comprises spectrin-like repeat 18 of dystrophin. In some embodiments, the micro-dystrophin protein further comprises spectrin-like repeat 19 of dystrophin. In some embodiments, the micro-dystrophin protein further comprises spectrin-like repeat 18 and spectrin-like repeat 19 of dystrophin. In some embodiments, the micro-dystrophin protein does not contain spectrin-like repeat 2 and/or spectrin-like repeat 3 of dystrophin. In some embodiments, the micro-dystrophin protein does not comprise spectrin-like repeat 2. In some embodiments, the micro-dystrophin protein does not comprise spectrin-like repeat 3. In some embodiments, the micro-dystrophin protein does not comprise spectrin-like repeat 2 and spectrin-like repeat 3.

In some embodiments, a micro-dystrophin protein optimized for cardiac muscle comprises one or more of: spectrin-like repeat 16, hinge domain 3, spectrin-like repeat 21, spectrin-like repeat 22, spectrin-like repeat 24, hinge domain 4, and dystroglycan binding site of dystrophin. In some embodiments, a micro-dystrophin protein optimized for cardiac muscle comprises spectrin-like repeat 24 and hinge domain 4. In some embodiments, a micro-dystrophin protein optimized for cardiac muscle comprises spectrin-like repeat 24 and the dystroglycan binding site of dystrophin. In some embodiments, a micro-dystrophin protein optimized for cardiac muscle comprises hinge domain 4 and the dystroglycan binding site of dystrophin. In some embodiments, a micro-dystrophin protein optimized for cardiac muscle comprises hinge domain 3, spectrin-like repeat 21, spectrin-like repeat 22, hinge domain 4 and the dystroglycan binding site of dystrophin. In some embodiments, a micro-dystrophin protein optimized for cardiac muscle comprises spectrin-like repeat 22, hinge domain 4 and the dystroglycan binding site of dystrophin. In some embodiments, a micro-dystrophin protein optimized for cardiac muscle comprises spectrin-like repeat 16, spectrin-like repeat 21, spectrin-like repeat 22, hinge domain 4 and the dystroglycan binding site of dystrophin.

In some embodiments, a micro-dystrophin protein optimized for cardiac muscle comprises one or more syntrophin binding domains of dystrophin.

In some embodiments, a micro-dystrophin protein optimized for cardiac muscle comprises one or more coiled coil domains of dystrophin.

In some embodiments, a micro-dystrophin protein optimized for cardiac muscle comprises spectrin-like repeat 1 directly coupled to spectrin-like repeat 17.

In some embodiments, a micro-dystrophin protein optimized for cardiac muscle comprises spectrin-like repeat 19 directly coupled to spectrin-like repeat 24. In some embodiments, a micro-dystrophin protein optimized for cardiac muscle comprises spectrin-like repeat 19 directly coupled to hinge domain 4.

In some embodiments, a micro-dystrophin protein optimized for cardiac muscle comprises spectrin-like repeat 17 directly coupled to hinge domain 3. In some embodiments, a micro-dystrophin protein optimized for cardiac muscle comprises spectrin-like repeat 17 directly coupled to spectrin-like repeat 22.

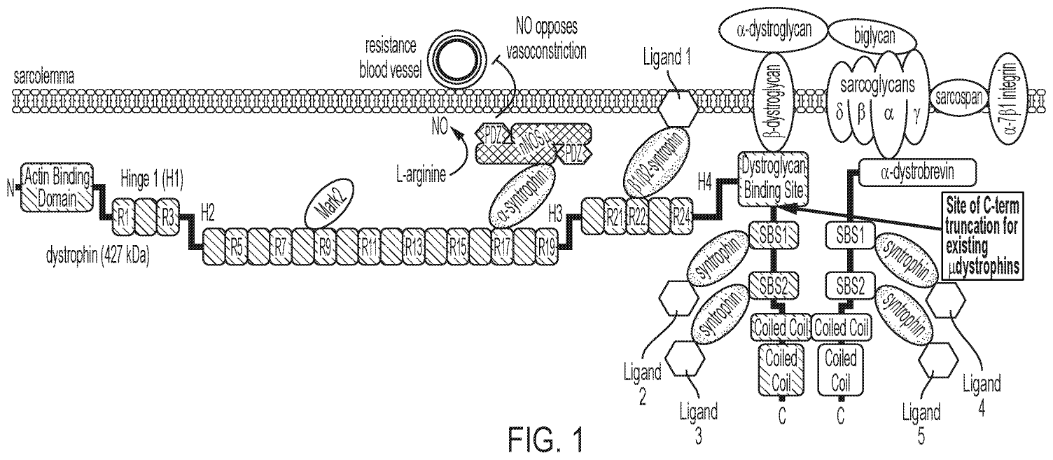

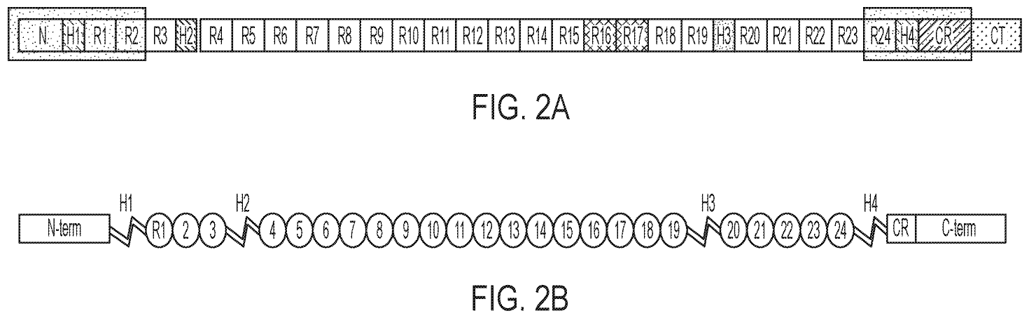

In some embodiments, a micro-dystrophin protein optimized for cardiac muscle comprises the region of dystrophin that is C-terminal to the cysteine-rich portion of the C-terminal region (FIGS. 1 and 2). In some embodiments, a micro-dystrophin protein optimized for cardiac muscle comprises the region of dystrophin that is C-terminal to the dystroglycan binding site of dystrophin.

In some embodiments, a micro-dystrophin protein optimized for cardiac muscle comprises the region of dystrophin from spectrin-like repeat 24 to the C-terminus. In some embodiments, a micro-dystrophin protein optimized for cardiac muscle comprises the region of dystrophin from spectrin-like repeat 24 to the end of the proline rich region following the first coiled coil domain.

In some embodiments, a micro-dystrophin protein optimized for cardiac muscle comprises the region of dystrophin from hinge domain 4 to the C-terminus. In some embodiments, a micro-dystrophin protein optimized for cardiac muscle comprises the region of dystrophin from hinge domain 4 to the end of the proline rich region following the first coiled coil domain. In some embodiments, a micro-dystrophin protein optimized for cardiac muscle comprises the region of dystrophin from hinge domain 4 to the end of the second syntrophin binding domain.

Domains of the dystrophin protein are known in the art. Exemplary sequences of domains of the dystrophin protein or combinations thereof are provided in Example 1. In some embodiments, a micro-dystrophin protein of the disclosure comprises an amino-terminal actin-binding domain and hinge domain 1 region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 40. In some embodiments, a micro-dystrophin protein of the disclosure comprises a spectrin-like repeat 1 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 41. In some embodiments, a micro-dystrophin protein of the disclosure comprises a spectrin-like repeat 2 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 42. In some embodiments, a micro-dystrophin protein of the disclosure comprises a spectrin-like repeat 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 43. In some embodiments, a micro-dystrophin protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44. In some embodiments, a micro-dystrophin protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44 without the linker sequence. In some embodiments, a micro-dystrophin protein of the disclosure comprises a spectrin-like repeat 17 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 45. In some embodiments, a micro-dystrophin protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 46. In some embodiments, a micro-dystrophin protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 81. In some embodiments, a micro-dystrophin protein of the disclosure comprises a hinge domain 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 119. In some embodiments, a micro-dystrophin protein of the disclosure comprises a spectrin-like repeat 21 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 120. In some embodiments, a micro-dystrophin protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 121. In some embodiments, a micro-dystrophin protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 121 without the linker sequence. In some embodiments, a micro-dystrophin protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 47. In some embodiments, a micro-dystrophin protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 82. In some embodiments, a micro-dystrophin protein of the disclosure comprises a spectrin-like repeat 24 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 48. In some embodiments, a micro-dystrophin protein of the disclosure comprises a hinge domain 4 and dystroglycan binding site (also referred to herein as “trCterm”) comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 49. In some embodiments, a micro-dystrophin protein of the disclosure comprises a syntrophin binding region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 50. In some embodiments, a micro-dystrophin protein of the disclosure comprises a 1^{st} coiled coil region and proline rich region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51. In some embodiments, a micro-dystrophin protein of the disclosure comprises a 1^{st} coiled coil region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51 without the proline rich region. In some embodiments, a micro-dystrophin protein of the disclosure comprises a 2^{nd} coiled coil region to end of dystrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 52. In some embodiments, a micro-dystrophin protein of the disclosure comprises a an amino acid sequence that has up to 10 (e.g., 1, 2, 3, 4, 5, 6, 7, 8, 9, or 10) amino acid additions or deletions relative to a sequence provided herein (e.g., any one of SEQ ID NOs. 40-52, 81, 82, and 119-121).

Non-limiting examples of human micro-dystrophin proteins optimized for cardiac muscle are described below.

Nterm-R1_R17-R19_R24 to trCterm:
(SEQ ID NO: 1)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDREDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQWQRKIDETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHELSTSVQGPWERAISPNKV
PYYINHETQTTCWDHPKMTELYQSLADLNNVRFSAYRTAMKLRRLQKALCLDLLSLSAACDALDQHNLKQNDQPMDI
LQIINCLTTIYDRLEQEHNNLVNVPLCVDMCLNWLLNVYDTGRIGRIRVLSFKTGIISLCKAHLEDKYRYLFKQVAS
STGFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETP
Nterm-R1_R17-R19_R24 to trCterm:
(SEQ ID NO: 86)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQWQRKIDETLERLQELQEATDELDLKLRQAEVIKGSWQPVGDLLIDSLQDHLEKVKALRGEIAPLKENVSHVN
DLARQLTTLGIQLSPYNLSTLEDLNTRWKLLQVAVEDRVRQLHEAHRDFGPASQHELSTSVQGPWERAISPNKVPYY
INHETQTTCWDHPKMTELYQSLADLNNVRFSAYRTAMKLRRLQKALCLDLLSLSAACDALDQHNLKQNDQPMDILQI
INCLTTIYDRLEQEHNNLVNVPLCVDMCLNWLLNVYDTGRIGRIRVLSFKTGIISLCKAHLEDKYRYLFKQVASSTG
FCDQRRLGLLLHDSIQIPRQLGEVASFGGSNIEPSVRSCFQFANNKPEIEAALFLDWMRLEPQSMVWLPVLHRVAAA
ETAKHQAKCNICKECPIIGFRYRSLKHFNYDICQSCFFSGRVAKGHKMHYPMVEYCTPTTSGEDVRDFAKVLKNKFR
TKRYFAKHPRMGYLPVQTVLEGDNMETP
Nterm-R1_R17-R19_H4 to 1st coiled coil and proline rich region that follows:
(SEQ ID NO: 2)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRFDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQGPASQHELSTSVQGPWERAISPNKVPYYINHETQTTCWDHPKMTELYQSLADLNNVRFSAYRTAMKLRR
LQKALCLDLLSLSAACDALDQHNLKQNDQPMDILQIINCLTTIYDRLEQEHNNLVNVPLCVDMCLNWLLNVYDTGRT
GRIRVLSFKTGIISLCKAHLEDKYRYLFKQVASSTGFCDQRRLGLLLHDSIQIPRQLGEVASEGGSNIEPSVRSCFQ
FANNKPEIEAALFLDWMRLEPQSMVWLPVLHRVAAAETAKHQAKCNICKECPIIGFRYRSLKHFNYDICQSCFFSGR
VAKGHKMHYPMVEYCTPTTSGEDVRDFAKVLKNKFRTKRYFAKHPRMGYLPVQTVLEGDNMETPVTLINFWPVDSAP
ASSPQLSHDDTHSRIEHYASRLAEMENSNGSYLNDSISPNESIDDEHLLIQHYCQSLNQDSPLSQPRSPAQILISLE
SEERGELERILADLEEENRNLQAEYDRLKQQHEHKGLSPLPSPPEMMPTSPQSP
Nterm-R1_R17-R19_H4 to 1st coiled coil and proline rich region that follows:
(SEQ ID NO: 87)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQGPASQHELSTSVQGPWERAISPNKVPYYINHETQTTCWDHPKMTELYQSLADLNNVRESAYRTAMKLRRLQK
ALCLDLLSLSAACDALDQHNLKQNDQPMDILQIINCLTTIYDRLEQEHNNLVNVPLCVDMCLNWLLNVYDTGRIGRI
RVLSFKTGIISLCKAHLEDKYRYLFKQVASSTGFCDQRRLGLLLHDSIQIPRQLGEVASFGGSNIEPSVRSCFQFAN
NKPEIEAALFLDWMRLEPQSMVWLPVLHRVAAAETAKHQAKCNICKECPIIGFRYRSLKHFNYDICQSCFFSGRVAK
GHKMHYPMVEYCTPTTSGEDVRDFAKVLKNKFRTKRYFAKHPRMGYLPVQTVLEGDNMETPVTLINFWPVDSAPASS
PQLSHDDTHSRIEHYASRLAEMENSNGSYLNDSISPNESIDDEHLLIQHYCQSLNQDSPLSQPRSPAQILISLESEE
RGELERILADLEEENRNLQAEYDRLKQQHEHKGLSPLPSPPEMMPTSPQSP
Nterm-R1_R17-R19_H4 to end of syntrophin region:
(SEQ ID NO: 3)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRFDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQGPASQHELSTSVQGPWERAISPNKVPYYINHETQTTCWDHPKMTELYQSLADLNNVRFSAYRTAMKLRR
LQKALCLDLLSLSAACDALDQHNLKQNDQPMDILQIINCLTTIYDRLEQEHNNLVNVPLCVDMCLNWLLNVYDTGRT
GRIRVLSFKTGIISLCKAHLEDKYRYLFKQVASSTGFCDQRRLGLLLHDSIQIPRQLGEVASEGGSNIEPSVRSCFQ
FANNKPEIEAALFLDWMRLEPQSMVWLPVLHRVAAAETAKHQAKCNICKECPIIGFRYRSLKHENYDICQSCFFSGR
VAKGHKMHYPMVEYCTPTTSGEDVRDFAKVLKNKFRTKRYFAKHPRMGYLPVQTVLEGDNMETPVTLINFWPVDSAP
ASSPQLSHDDTHSRIEHYASRLAEMENSNGSYLNDSISPNESIDDEHLLIQHYCQSLNQDSPLSQPRS
Nterm-R1_R17-R19_H4 to end of syntrophin region:
(SEQ ID NO: 88)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLILGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQGPASQHELSTSVQGPWERAISPNKVPYYINHETQTTCWDHPKMTELYQSLADLNNVRFSAYRTAMKLRRLQK
ALCLDLLSLSAACDALDQHNLKQNDQPMDILQIINCLTTIYDRLEQEHNNLVNVPLCVDMCLNWLLNVYDTGRIGRI
RVLSFKTGIISLCKAHLEDKYRYLFKQVASSTGFCDQRRLGLLLHDSIQIPRQLGEVASEGGSNIEPSVRSCFQFAN
NKPEIEAALFLDWMRLEPQSMVWLPVLHRVAAAETAKHQAKCNICKECPIIGFRYRSLKHFNYDICQSCFFSGRVAK
GHKMHYPMVEYCTPTTSGEDVRDFAKVLKNKFRTKRYFAKHPRMGYLPVQTVLEGDNMETPVTLINFWPVDSAPASS
PQLSHDDTHSRIEHYASRLAEMENSNGSYLNDSISPNESIDDEHLLIQHYCOSLNODSPLSQPRS
N-terminus through R1_R17-R19_R24 to 1st coiled coil and proline rich region that
follows:
(SEQ ID NO: 4)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDREDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQWQRKIDETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHFLSTSVQGPWERAISPNKV
PYYINHETQTTCWDHPKMTELYQSLADLNNVRFSAYRTAMKLRRLQKALCLDLLSLSAACDALDQHNLKQNDQPMDI
LQIINCLTTIYDRLEQEHNNLVNVPLCVDMCLNWLLNVYDTGRTGRIRVLSFKTGIISLCKAHLEDKYRYLFKQVAS
STGFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYASRLAEMENSNGSYL
NDSISPNESIDDEHLLIQHYCQSLNQDSPLSQPRSPAQILISLESEERGELERILADLEEENRNLQAEYDRLKQQHE
HKGLSPLPSPPEMMPTSPQSP
N-terminus through R1_R17-R19_R24 to 1st coiled coil and proline rich region that
follows:
(SEQ ID NO: 89)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQWQRKIDETLERLQELQEATDELDLKLRQAEVIKGSWQPVGDLLIDSLQDHLEKVKALRGEIAPLKENVSHVN
DLARQLTTLGIQLSPYNLSTLEDLNTRWKLLQVAVEDRVRQLHEAHRDFGPASQHELSTSVQGPWERAISPNKVPYY
INHETQTTCWDHPKMTELYQSLADLNNVRFSAYRTAMKLRRLQKALCLDLLSLSAACDALDQHNLKQNDQPMDILQI
INCLTTIYDRLEQEHNNLVNVPLCVDMCLNWLLNVYDTGRTGRIRVLSFKTGIISLCKAHLEDKYRYLFKQVASSTG
FCDQRRLGLLLHDSIQIPRQLGEVASFGGSNIEPSVRSCFQFANNKPEIEAALFLDWMRLEPQSMVWLPVLHRVAAA
ETAKHQAKCNICKECPIIGFRYRSLKHFNYDICQSCFFSGRVAKGHKMHYPMVEYCTPTTSGEDVRDFAKVLKNKFR
TKRYFAKHPRMGYLPVQTVLEGDNMETPVTLINFWPVDSAPASSPQLSHDDTHSRIEHYASRLAEMENSNGSYLNDS
ISPNESIDDEHLLIQHYCQSLNQDSPLSQPRSPAQILISLESEERGELERILADLEEENRNLQAEYDRLKQQHEHKG
LSPLPSPPEMMPTSPQSP
N-terminus through R1_R17-R19_H4 to end of dystrophin:
(SEQ ID NO: 5)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRFDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQGPASQHELSTSVQGPWERAISPNKVPYYINHETQTTCWDHPKMTELYQSLADLNNVRESAYRTAMKLRR
LQKALCLDLLSLSAACDALDQHNLKQNDQPMDILQIINCLTTIYDRLEQEHNNLVNVPLCVDMCLNWLLNVYDTGRT
GRIRVLSFKTGIISLCKAHLEDKYRYLFKQVASSTGFCDQRRLGLLLHDSIQIPRQLGEVASEGGSNIEPSVRSCFQ
FANNKPEIEAALFLDWMRLEPQSMVWLPVLHRVAAAETAKHQAKCNICKECPIIGFRYRSLKHFNYDICQSCFFSGR
VAKGHKMHYPMVEYCTPTTSGEDVRDFAKVLKNKFRTKRYFAKHPRMGYLPVQTVLEGDNMETPVTLINFWPVDSAP
ASSPQLSHDDTHSRIEHYASRLAEMENSNGSYLNDSISPNESIDDEHLLIQHYCQSLNQDSPLSQPRSPAQILISLE
SEERGELERILADLEEENRNLQAEYDRLKQQHEHKGLSPLPSPPEMMPTSPQSPRDAELIAEAKLLRQHKGRLEARM
QILEDHNKQLESQLHRLRQLLEQPQAEAKVNGTTVSSPSTSLQRSDSSQPMLLRVVGSQTSDSMGEEDLLSPPQDTS
TGLEEVMEQLNNSFPSSRGRNTPGKPMREDTM
N-terminus through R1_R17-R19_H4 to end of dystrophin:
(SEQ ID NO: 90)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQGPASQHELSTSVQGPWERAISPNKVPYYINHETQTTCWDHPKMTELYQSLADLNNVRFSAYRTAMKLRRLQK
ALCLDLLSLSAACDALDQHNLKQNDQPMDILQIINCLTTIYDRLEQEHNNLVNVPLCVDMCLNWLLNVYDTGRTGRI
RVLSFKTGIISLCKAHLEDKYRYLFKQVASSTGFCDQRRLGLLLHDSIQIPRQLGEVASFGGSNIEPSVRSCFQFAN
NKPEIEAALFLDWMRLEPQSMVWLPVLHRVAAAETAKHQAKCNICKECPIIGFRYRSLKHFNYDICQSCFFSGRVAK
GHKMHYPMVEYCTPTTSGEDVRDFAKVLKNKFRTKRYFAKHPRMGYLPVQTVLEGDNMETPVTLINFWPVDSAPASS
PQLSHDDTHSRIEHYASRLAEMENSNGSYLNDSISPNESIDDEHLLIQHYCQSLNQDSPLSQPRSPAQILISLESEE
RGELERILADLEEENRNLQAEYDRLKQQHEHKGLSPLPSPPEMMPTSPQSPRDAELIAEAKLLRQHKGRLEARMQIL
EDHNKQLESQLHRLRQLLEQPQAEAKVNGTTVSSPSTSLQRSDSSQPMLLRVVGSQTSDSMGEEDLLSPPQDTSTGL
EEVMEQLNNSFPSSRGRNTPGKPMREDTM
N-terminus through R1_R17-R19_R24 to end of dystrophin:
(SEQ ID NO: 6)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDREDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKNILSEFQRDLNEFVLWLEEADNIASIPLEPGKEQQLKEKLEQVKLLVEELPL
RQGILKQLNETGGPVLVSAPISPEEQDKLENKLKQTNLQWIKVSRALPEKQGEIEAQIKDLGQLEKKLEDLEEQLNH
LLLWLSPIRNQLEIYNQPNQEGPFDVKETEIAVQAKQPDVEEILSKGQHLYKEKPATQPVKRKLEDLSSEWKAVNRL
LQELRAKQWQRKIDETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHELSTSVQGPWERAISPNKV
PYYINHETQTTCWDHPKMTELYQSLADLNNVRFSAYRTAMKLRRLQKALCLDLLSLSAACDALDQHNLKQNDQPMDI
LQIINCLTTIYDRLEQEHNNLVNVPLCVDMCLNWLLNVYDTGRTGRIRVLSFKTGIISLCKAHLEDKYRYLFKQVAS
STGFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYASRLAEMENSNGSYL
NDSISPNESIDDEHLLIQHYCQSLNQDSPLSQPRSPAQILISLESEERGELERILADLEEENRNLQAEYDRLKQQHE
HKGLSPLPSPPEMMPTSPQSPRDAELIAEAKLLRQHKGRLEARMQILEDHNKQLESQLHRLRQLLEQPQAEAKVNGT
TVSSPSTSLQRSDSSQPMLLRVVGSQTSDSMGEEDLLSPPQDTSTGLEEVMEQLNNSFPSSRGRNTPGKPMREDTM
N-terminus through R1_R17-R19_R24 to end of dystrophin:
(SEQ ID NO: 91)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKNILSEFQRDLNEFVLWLEEADNIASIPLEPGKEQQLKEKLEQVKLLVEELPLRQG
ILKQLNETGGPVLVSAPISPEEQDKLENKLKQTNLQWIKVSRALPEKQGEIEAQIKDLGQLEKKLEDLEEQLNHLLL
WLSPIRNQLEIYNQPNQEGPFDVKETEIAVQAKQPDVEEILSKGQHLYKEKPATQPVKRKLEDLSSEWKAVNRLLQE
LRAKQWQRKIDETLERLQELQEATDELDLKLRQAEVIKGSWQPVGDLLIDSLQDHLEKVKALRGEIAPLKENVSHVN
DLARQLTTLGIQLSPYNLSTLEDLNTRWKLLQVAVEDRVRQLHEAHRDFGPASQHELSTSVQGPWERAISPNKVPYY
INHETQTTCWDHPKMTELYQSLADLNNVRFSAYRTAMKLRRLQKALCLDLLSLSAACDALDQHNLKQNDQPMDILQI
INCLITIYDRLEQEHNNLVNVPLCVDMCLNWLLNVYDTGRIGRIRVLSFKTGIISLCKAHLEDKYRYLFKQVASSTG
FCDQRRLGLLLHDSIQIPRQLGEVASFGGSNIEPSVRSCFQFANNKPEIEAALFLDWMRLEPQSMVWLPVLHRVAAA
ETAKHQAKCNICKECPIIGFRYRSLKHFNYDICQSCFFSGRVAKGHKMHYPMVEYCTPTTSGEDVRDFAKVLKNKER
TKRYFAKHPRMGYLPVQTVLEGDNMETPVTLINFWPVDSAPASSPQLSHDDTHSRIEHYASRLAEMENSNGSYLNDS
ISPNESIDDEHLLIQHYCQSLNQDSPLSQPRSPAQILISLESEERGELERILADLEEENRNLQAEYDRLKQQHEHKG
LSPLPSPPEMMPTSPQSPRDAELIAEAKLLRQHKGRLEARMQILEDHNKQLESQLHRLRQLLEQPQAEAKVNGTTVS
SPSTSLQRSDSSQPMLLRVVGSQTSDSMGEEDLLSPPQDTSTGLEEVMEQLNNSFPSSRGRNTPGKPMREDTM
Nterm-R1_R17_H3_R21-R22_H4 to end of syntrophin region
(SEQ ID NO: 106)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRFDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKQPDLAPGLTTIGASPTQTVTLVTQPVVTKETAISKLEMPSSLMLEVPKDSTQ
WLEAKEEAEQVLGQARAKLESWKEGPYTVDAIQKKITETKQLAKDLRQWQTNVDVANDLALKLLRDYSADDTRKVHM
ITENINASWRSIHKRVSEREAALEETHRLLQQFPLDLEKFLAWLTEAETTANVLQDATRKERLLEDSKGVKELMKQW
QDLQGEIEAHTDVYHNLDENSQKILRSLEGSDDAVLLQRRLDNMNEKWSELRKKSLNIRSHLEASSDQWKRLGPASQ
HELSTSVQGPWERAISPNKVPYYINHETQTTCWDHPKMTELYQSLADLNNVRFSAYRTAMKLRRLQKALCLDLLSLS
AACDALDQHNLKQNDQPMDILQIINCLTTIYDRLEQEHNNLVNVPLCVDMCLNWLLNVYDTGRTGRIRVLSFKTGII
SLCKAHLEDKYRYLFKQVASSTGFCDQRRLGLLLHDSIQIPRQLGEVASFGGSNIEPSVRSCFQFANNKPEIEAALF
LDWMRLEPQSMVWLPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSAPASSPQLSHDDTHS
RIEHYASRLAEMENSNGSYLNDSISPNESIDDEHLLIQHYCQSLNODSPLSQPRS
Nterm-R1_R17_R22_H4 to 1st coiled coil
(SEQ ID NO: 107)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDRFDRSVEKWRR
FHYDIKIFNQWLTEAEQFLRKTQIPENWEHAKYKWYLKELQDGIGQRQTVVRTLNATGEEIIQQSSKTDASILQEKL
GSLNLRWQEVCKQLSDRKKRLEEQKEETHRLLQQFPLDLEKFLAWLTEAETTANVLQDATRKERLLEDSKGVKELMK
QWQDLQGEIEAHTDVYHNLDENSQKILRSLEGSDDAVLLQRRLDNMNFKWSELRKKSLNIRSHLEASSDQWKRLGPA
SQHELSTSVQGPWERAISPNKVPYYINHETQTTCWDHPKMTELYQSLADLNNVRFSAYRTAMKLRRLQKALCLDLLS
LSAACDALDQHNLKQNDQPMDILQIINCLTTIYDRLEQEHNNLVNVPLCVDMCLNWLLNVYDTGRIGRIRVLSFKTG
IISLCKAHLEDKYRYLFKQVASSTGFCDQRRLGLLLHDSIQIPRQLGEVASEGGSNIEPSVRSCFQFANNKPEIEAA
LFLDWMRLEPQSMVWLPVLHRVAAAETAKHQAKCNICKECPIIGFRYRSLKHFNYDICQSCFFSGRVAKGHKMHYPM
VEYCTPTTSGEDVRDFAKVLKNKFRTKRYFAKHPRMGYLPVQTVLEGDNMETPVTLINFWPVDSAPASSPQLSHDDT
HSRIEHYASRLAEMENSNGSYLNDSISPNESIDDEHLLIQHYCQSLNQDSPLSQPRSPAQILISLESEERGELERIL
ADLEEENRNLQAEYDRLKQQHEHKGLS
Nterm-R1_R16-R17_R21-R22_H4 to end of syntrophin region
(SEQ ID NO: 108)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDHTVREETMMVM
TEDMPLEISYVPSTYLTEITHVSQALLEVEQLLNAPDLCAKDFEDLFKQEESLKNIKDSLQQSSGRIDIIHSKKTAA
LQSATPVERVKLQEALSQLDFQWEKVNKMYKDRQGREDRSVEKWRRFHYDIKIFNQWLTEAEQFLRKTQIPENWEHA
KYKWYLKELQDGIGQRQTVVRTLNATGEEIIQQSSKTDASILQEKLGSLNLRWQEVCKQLSDRKKRLEEQKKDSTQW
LEAKEEAEQVLGQARAKLESWKEGPYTVDAIQKKITETKQLAKDLRQWQTNVDVANDLALKLLRDYSADDTRKVHMI
TENINASWRSIHKRVSEREAALEETHRLLQQFPLDLEKFLAWLTEAETTANVLQDATRKERLLEDSKGVKELMKQWQ
DLQGEIEAHTDVYHNLDENSQKILRSLEGSDDAVLLQRRLDNMNEKWSELRKKSLNIRSHLEASSDQWKRLGPASQH
FLSTSVQGPWERAISPNKVPYYINHETQTTCWDHPKMTELYQSLADLNNVRFSAYRTAMKLRRLQKALCLDLLSLSA
ACDALDQHNLKQNDQPMDILQIINCLTTIYDRLEQEHNNLVNVPLCVDMCLNWLLNVYDTGRIGRIRVLSFKTGIIS
LCKAHLEDKYRYLFKQVASSTGFCDQRRLGLLLHDSIQIPRQLGEVASFGGSNIEPSVRSCFQFANNKPEIEAALFL
DWMRLEPQSMVWLPVLHRVAAAETAKHQAKCNICKECPIIGFRYRSLKHFNYDICQSCFFSGRVAKGHKMHYPMVEY
CTPTTSGEDVRDFAKVLKNKFRTKRYFAKHPRMGYLPVQTVLEGDNMETPVTLINFWPVDSAPASSPQLSHDDTHSR
IEHYASRLAEMENSNGSYLNDSISPNESIDDEHLLIQHYCQSLNQDSPLSQPRS
A micro-dystrophin protein optimized for cardiac muscle is further understood to
include proteins that are variants of any one of SEQ ID Nos. 1-6, 86-91, and
106-108. Variant polypeptides include polypeptides that differ by one or more
(e.g., 1, 2, 3, 4, 5, 6, 7, 8, 9, 10, 11, 12, 13, 14, 15, 16, 17, 18, 19, 20, or
more) amino acid substitutions, additions, or deletions, and

will, therefore, include amino acid sequences that differ from the amino acid sequences designated in any one of SEQ ID Nos. 1-6, 86-91, and 106-108.

In some embodiments, a micro-dystrophin protein optimized for cardiac muscle comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 86-91, and 106-108.

In some embodiments, a micro-dystrophin protein optimized for cardiac muscle consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 86-91, and 106-108.

In some embodiments, a micro-dystrophin protein optimized for cardiac muscle consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 86-91, and 106-108.

In some embodiments, functional variants or modified forms of micro-dystrophin proteins optimized for cardiac muscle include fusion proteins having at least a portion of the micro-dystrophin protein and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

The disclosure also provides nucleic acids encoding any of the micro-dystrophins optimized for cardiac muscle described herein. Such nucleic acids may be DNA or RNA molecules. These nucleic acids may be used, for example, in methods for making micro-dystrophins or as direct therapeutic agents in a gene therapy approach.

Non-limiting examples of nucleotide sequences encoding human micro-dystrophin proteins optimized for cardiac muscle are described below.

Nterm-R1_R17-R19_R24 to trCterm:
(SEQ ID NO: 7)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTIGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGTGGCAGAGAAAAATAGATGAGACCCTTGAAAGACTCCGGGAACTTCAAGAGGC
CACGGATGAGCTGGACCTCAAGCTGCGCCAAGCTGAGGTGATCAAGGGATCCTGGCAGCCCGTGGGCGATCTCCTCA
TTGACTCTCTCCAAGATCACCTCGAGAAAGTCAAGGCACTTCGAGGAGAAATTGCGCCTCTGAAAGAGAACGTGAGC
CACGTCAATGACCTTGCTCGCCAGCTTACCACTTTGGGCATTCAGCTCTCACCGTATAACCTCAGCACTCTGGAAGA
CCTGAACACCAGATGGAAGCTTCTGCAGGTGGCCGTCGAGGACCGAGTCAGGCAGCTGCATGAAGCCCACAGGGACT
TTGGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTAGC
TGACCTGAATAATGTCAGATTCTCAGCTTATAGGACTGCCATGAAACTCCGAAGACTGCAGAAGGCCCTTTGCTTGG
ATCTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCTCT
CTGCGTGGATATGTGTCTGAACTGGCTGCTGAATGTTTATGATACGGGACGAACAGGGAGGATCCGTGTCCTGTCTT
TTAAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGTGA
AGTTGCATCCTTTGGGGGCAGTAACATTGAGCCAAGTGTCCGGAGCTGCTTCCAATTTGCTAATAATAAGCCAGAGA
TCGAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTACAG
GAGTCTAAAGCACTTTAATTATGACATCTGCCAAAGCTGCTTTTTTTCTGGTCGAGTTGCAAAAGGCCATAAAATGC
ACTATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGGGA
CAACATGGAAACTCCC
Nterm-R1_R17-R19_R24 to trCterm:
(SEQ ID NO: 92)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTTGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGTGGCAGAGAAAAATAGATGAGACCCTTGAAAGACTCCGGGAACTTCAAGAGGCCACGGATGA
GCTGGACCTCAAGCTGCGCCAAGCTGAGGTGATCAAGGGATCCTGGCAGCCCGTGGGCGATCTCCTCATTGACTCTC
TCCAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAACAC
CAGATGGAAGCTTCTGCAGGTGGCCGTCGAGGACCGAGTCAGGCAGCTGCATGAAGCCCACAGGGACTTTGGTCCAG
CATCTCAGCACTTTCTTTCCACGTCTGTCCAGGGTCCCTGGGAGAGAGCCATCTCGCCAAACAAAGTGCCCTACTAT
ATCAACCACGAGACTCAAACAACTTGCTGGGACCATCCCAAAATGACAGAGCTCTACCAGTCTTTAGCTGACCTGAA
TAATGTCAGATTCTCAGCTTATAGGACTGCCATGAAACTCCGAAGACTGCAGAAGGCCCTTTGCTTGGATCTCTTGA
GCCTGTCAGCTGCATGTGATGCCTTGGACCAGCACAACCTCAAGCAAAATGACCAGCCCATGGATATCCTGCAGATT
ATTAATTGTTTGACCACTATTTATGACCGCCTGGAGCAAGAGCACAACAATTTGGTCAACGTCCCTCTCTGCGTGGA
TATGTGTCTGAACTGGCTGCTGAATGTTTATGATACGGGACGAACAGGGAGGATCCGTGTCCTGTCTTTTAAAACTG
GCATCATTTCCCTGTGTAAAGCACATTTGGAAGACAAGTACAGATACCTTTTCAAGCAAGTGGCAAGTTCAACAGGA
TTTTGTGACCAGCGCAGGCTGGGCCTCCTTCTGCATGATTCTATCCAAATTCCAAGACAGTTGGGTGAAGTTGCATC
CTTTGGGGGCAGTAACATTGAGCCAAGTGTCCGGAGCTGCTTCCAATTTGCTAATAATAAGCCAGAGATCGAAGCGG
CCCTCTTCCTAGACTGGATGAGACTGGAACCCCAGTCCATGGTGTGGCTGCCCGTCCTGCACAGAGTGGCTGCTGCA
GAAACTGCCAAGCATCAGGCCAAATGTAACATCTGCAAAGAGTGTCCAATCATTGGATTCAGGTACAGGAGTCTAAA
GCACTTTAATTATGACATCTGCCAAAGCTGCTTTTTTTCTGGTCGAGTTGCAAAAGGCCATAAAATGCACTATCCCA
TGGTGGAATATTGCACTCCGACTACATCAGGAGAAGATGTTCGAGACTTTGCCAAGGTACTAAAAAACAAATTTCGA
ACCAAAAGGTATTTTGCGAAGCATCCCCGAATGGGCTACCTGCCAGTGCAGACTGTCTTAGAGGGGGACAACATGGA
AACTCCC
Nterm-R1_R17-R19_H4 to 1st coiled coil and proline rich region that follows:
(SEQ ID NO: 8)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTTGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGGGTCCAGCATCTCAGCACTTTCTTTCCACGTCTGTCCAGGGTCCCTGGGAGAG
AGCCATCTCGCCAAACAAAGTGCCCTACTATATCAACCACGAGACTCAAACAACTTGCTGGGACCATCCCAAAATGA
CAGAGCTCTACCAGTCTTTAGCTGACCTGAATAATGTCAGATTCTCAGCTTATAGGACTGCCATGAAACTCCGAAGA
CTGCAGAAGGCCCTTTGCTTGGATCTCTTGAGCCTGTCAGCTGCATGTGATGCCTTGGACCAGCACAACCTCAAGCA
AAATGACCAGCCCATGGATATCCTGCAGATTATTAATTGTTTGACCACTATTTATGACCGCCTGGAGCAAGAGCACA
ACAATTTGGTCAACGTCCCTCTCTGCGTGGATATGTGTCTGAACTGGCTGCTGAATGTTTATGATACGGGACGAACA
GGGAGGATCCGTGTCCTGTCTTTTAAAACTGGCATCATTTCCCTGTGTAAAGCACATTTGGAAGACAAGTACAGATA
CCTTTTCAAGCAAGTGGCAAGTTCAACAGGATTTTGTGACCAGCGCAGGCTGGGCCTCCTTCTGCATGATTCTATCC
AAATTCCAAGACAGTTGGGTGAAGTTGCATCCTTTGGGGGCAGTAACATTGAGCCAAGTGTCCGGAGCTGCTTCCAA
TTTGCTAATAATAAGCCAGAGATCGAAGCGGCCCTCTTCCTAGACTGGATGAGACTGGAACCCCAGTCCATGGTGTG
GCTGCCCGTCCTGCACAGAGTGGCTGCTGCAGAAACTGCCAAGCATCAGGCCAAATGTAACATCTGCAAAGAGTGTC
CAATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGAGA
CTTTGCCAAGGTACTAAAAAACAAATTTCGAACCAAAAGGTATTTTGCGAAGCATCCCCGAATGGGCTACCTGCCAG
TGCAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATGGA
AAACAGCAATGGATCTTATCTAAATGATAGCATCTCTCCTAATGAGAGCATAGATGATGAACATTTGTTAATCCAGC
ATTACTGCCAAAGTTTGAACCAGGACTCCCCCCTGAGCCAGCCTCGTAGTCCTGCCCAGATCTTGATTTCCTTAGAG
AGTGAGGAAAGAGGGGAGCTAGAGAGAATCCTAGCAGATCTTGAGGAAGAAAACAGGAATCTGCAAGCAGAATATGA
CCGTCTAAAGCAGCAGCACGAACATAAAGGCCTGTCCCCACTGCCGTCCCCTCCTGAAATGATGCCCACCTCTCCCC
AGAGTCCC
Nterm-R1_R17-R19_H4 to 1st coiled coil and proline rich region that follows:
(SEQ ID NO: 93)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTIGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGGGTCCAGCATCTCAGCACTTTCTTTCCACGTCTGTCCAGGGTCCCTGGGAGAGAGCCATCTC
GCCAAACAAAGTGCCCTACTATATCAACCACGAGACTCAAACAACTTGCTGGGACCATCCCAAAATGACAGAGCTCT
ACCAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGACCA
GCCCATGGATATCCTGCAGATTATTAATTGTTTGACCACTATTTATGACCGCCTGGAGCAAGAGCACAACAATTTGG
TCAACGTCCCTCTCTGCGTGGATATGTGTCTGAACTGGCTGCTGAATGTTTATGATACGGGACGAACAGGGAGGATC
CGTGTCCTGTCTTTTAAAACTGGCATCATTTCCCTGTGTAAAGCACATTTGGAAGACAAGTACAGATACCTTTTCAA
GCAAGTGGCAAGTTCAACAGGATTTTGTGACCAGCGCAGGCTGGGCCTCCTTCTGCATGATTCTATCCAAATTCCAA
GACAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCCGT
CCTGCACAGAGTGGCTGCTGCAGAAACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGCTTTTTTTCTGGTCGAGTTGCAAAA
GGCCATAAAATGCACTATCCCATGGTGGAATATTGCACTCCGACTACATCAGGAGAAGATGTTCGAGACTTTGCCAA
GGTACTAAAAAACAAATTTCGAACCAAAAGGTATTTTGCGAAGCATCCCCGAATGGGCTACCTGCCAGTGCAGACTG
TCTTAGAGGGGGACAACATGGAAACTCCCGTTACTCTGATCAACTTCTGGCCAGTAGATTCTGCGCCTGCCTCGTCC
CCTCAGCTTTCACACGATGATACTCATTCACGCATTGAACATTATGCTAGCAGGCTAGCAGAAATGGAAAACAGCAA
TGGATCTTATCTAAATGATAGCATCTCTCCTAATGAGAGCATAGATGATGAACATTTGTTAATCCAGCATTACTGCC
AAAGTTTGAACCAGGACTCCCCCCTGAGCCAGCCTCGTAGTCCTGCCCAGATCTTGATTTCCTTAGAGAGTGAGGAA
AGAGGGGAGCTAGAGAGAATCCTAGCAGATCTTGAGGAAGAAAACAGGAATCTGCAAGCAGAATATGACCGTCTAAA
GCAGCAGCACGAACATAAAGGCCTGTCCCCACTGCCGTCCCCTCCTGAAATGATGCCCACCTCTCCCCAGAGTCCC
Nterm-R1_R17-R19_H4 to end of syntrophin region:
(SEQ ID NO: 9)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTTGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGGGTCCAGCATCTCAGCACTTTCTTTCCACGTCTGTCCAGGGTCCCTGGGAGAG
AGCCATCTCGCCAAACAAAGTGCCCTACTATATCAACCACGAGACTCAAACAACTTGCTGGGACCATCCCAAAATGA
CAGAGCTCTACCAGTCTTTAGCTGACCTGAATAATGTCAGATTCTCAGCTTATAGGACTGCCATGAAACTCCGAAGA
CTGCAGAAGGCCCTTTGCTTGGATCTCTTGAGCCTGTCAGCTGCATGTGATGCCTTGGACCAGCACAACCTCAAGCA
AAATGACCAGCCCATGGATATCCTGCAGATTATTAATTGTTTGACCACTATTTATGACCGCCTGGAGCAAGAGCACA
ACAATTTGGTCAACGTCCCTCTCTGCGTGGATATGTGTCTGAACTGGCTGCTGAATGTTTATGATACGGGACGAACA
GGGAGGATCCGTGTCCTGTCTTTTAAAACTGGCATCATTTCCCTGTGTAAAGCACATTTGGAAGACAAGTACAGATA
CCTTTTCAAGCAAGTGGCAAGTTCAACAGGATTTTGTGACCAGCGCAGGCTGGGCCTCCTTCTGCATGATTCTATCC
AAATTCCAAGACAGTTGGGTGAAGTTGCATCCTTTGGGGGCAGTAACATTGAGCCAAGTGTCCGGAGCTGCTTCCAA
TTTGCTAATAATAAGCCAGAGATCGAAGCGGCCCTCTTCCTAGACTGGATGAGACTGGAACCCCAGTCCATGGTGTG
GCTGCCCGTCCTGCACAGAGTGGCTGCTGCAGAAACTGCCAAGCATCAGGCCAAATGTAACATCTGCAAAGAGTGTC
CAATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGAGA
CTTTGCCAAGGTACTAAAAAACAAATTTCGAACCAAAAGGTATTTTGCGAAGCATCCCCGAATGGGCTACCTGCCAG
TGCAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATGGA
AAACAGCAATGGATCTTATCTAAATGATAGCATCTCTCCTAATGAGAGCATAGATGATGAACATTTGTTAATCCAGC
ATTACTGCCAAAGTTTGAACCAGGACTCCCCCCTGAGCCAGCCTCGTAGT
Nterm-R1_R17-R19_H4 to end of syntrophin region:
(SEQ ID NO: 94)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTTGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGGGTCCAGCATCTCAGCACTTTCTTTCCACGTCTGTCCAGGGTCCCTGGGAGAGAGCCATCTC
GCCAAACAAAGTGCCCTACTATATCAACCACGAGACTCAAACAACTTGCTGGGACCATCCCAAAATGACAGAGCTCT
ACCAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGACCA
GCCCATGGATATCCTGCAGATTATTAATTGTTTGACCACTATTTATGACCGCCTGGAGCAAGAGCACAACAATTTGG
TCAACGTCCCTCTCTGCGTGGATATGTGTCTGAACTGGCTGCTGAATGTTTATGATACGGGACGAACAGGGAGGATC
CGTGTCCTGTCTTTTAAAACTGGCATCATTTCCCTGTGTAAAGCACATTTGGAAGACAAGTACAGATACCTTTTCAA
GCAAGTGGCAAGTTCAACAGGATTTTGTGACCAGCGCAGGCTGGGCCTCCTTCTGCATGATTCTATCCAAATTCCAA
GACAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCCGT
CCTGCACAGAGTGGCTGCTGCAGAAACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGCTTTTTTTCTGGTCGAGTTGCAAAA
GGCCATAAAATGCACTATCCCATGGTGGAATATTGCACTCCGACTACATCAGGAGAAGATGTTCGAGACTTTGCCAA
GGTACTAAAAAACAAATTTCGAACCAAAAGGTATTTTGCGAAGCATCCCCGAATGGGCTACCTGCCAGTGCAGACTG
TCTTAGAGGGGGACAACATGGAAACTCCCGTTACTCTGATCAACTTCTGGCCAGTAGATTCTGCGCCTGCCTCGTCC
CCTCAGCTTTCACACGATGATACTCATTCACGCATTGAACATTATGCTAGCAGGCTAGCAGAAATGGAAAACAGCAA
TGGATCTTATCTAAATGATAGCATCTCTCCTAATGAGAGCATAGATGATGAACATTTGTTAATCCAGCATTACTGCC
AAAGTTTGAACCAGGACTCCCCCCTGAGCCAGCCTCGTAGT
Nterm-R1_R17-R19_R24 to 1st coiled coil and proline rich region that follows:
(SEQ ID NO: 10)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTIGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGTGGCAGAGAAAAATAGATGAGACCCTTGAAAGACTCCGGGAACTTCAAGAGGC
CACGGATGAGCTGGACCTCAAGCTGCGCCAAGCTGAGGTGATCAAGGGATCCTGGCAGCCCGTGGGCGATCTCCTCA
TTGACTCTCTCCAAGATCACCTCGAGAAAGTCAAGGCACTTCGAGGAGAAATTGCGCCTCTGAAAGAGAACGTGAGC
CACGTCAATGACCTTGCTCGCCAGCTTACCACTTTGGGCATTCAGCTCTCACCGTATAACCTCAGCACTCTGGAAGA
CCTGAACACCAGATGGAAGCTTCTGCAGGTGGCCGTCGAGGACCGAGTCAGGCAGCTGCATGAAGCCCACAGGGACT
TTGGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTAGC
TGACCTGAATAATGTCAGATTCTCAGCTTATAGGACTGCCATGAAACTCCGAAGACTGCAGAAGGCCCTTTGCTTGG
ATCTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCTCT
CTGCGTGGATATGTGTCTGAACTGGCTGCTGAATGTTTATGATACGGGACGAACAGGGAGGATCCGTGTCCTGTCTT
TTAAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGTGA
AGTTGCATCCTTTGGGGGCAGTAACATTGAGCCAAGTGTCCGGAGCTGCTTCCAATTTGCTAATAATAAGCCAGAGA
TCGAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTACAG
GAGTCTAAAGCACTTTAATTATGACATCTGCCAAAGCTGCTTTTTTTCTGGTCGAGTTGCAAAAGGCCATAAAATGC
ACTATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGGGA
CAACATGGAAACTCCCGTTACTCTGATCAACTTCTGGCCAGTAGATTCTGCGCCTGCCTCGTCCCCTCAGCTTTCAC
ACGATGATACTCATTCACGCATTGAACATTATGCTAGCAGGCTAGCAGAAATGGAAAACAGCAATGGATCTTATCTA
AATGATAGCATCTCTCCTAATGAGAGCATAGATGATGAACATTTGTTAATCCAGCATTACTGCCAAAGTTTGAACCA
GGACTCCCCCCTGAGCCAGCCTCGTAGTCCTGCCCAGATCTTGATTTCCTTAGAGAGTGAGGAAAGAGGGGAGCTAG
AGAGAATCCTAGCAGATCTTGAGGAAGAAAACAGGAATCTGCAAGCAGAATATGACCGTCTAAAGCAGCAGCACGAA
CATAAAGGCCTGTCCCCACTGCCGTCCCCTCCTGAAATGATGCCCACCTCTCCCCAGAGTCCC
Nterm-R1_R17-R19_R24 to 1st coiled coil and proline rich region that follows:
(SEQ ID NO: 95)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTTGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGTGGCAGAGAAAAATAGATGAGACCCTTGAAAGACTCCGGGAACTTCAAGAGGCCACGGATGA
GCTGGACCTCAAGCTGCGCCAAGCTGAGGTGATCAAGGGATCCTGGCAGCCCGTGGGCGATCTCCTCATTGACTCTC
TCCAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAACAC
CAGATGGAAGCTTCTGCAGGTGGCCGTCGAGGACCGAGTCAGGCAGCTGCATGAAGCCCACAGGGACTTTGGTCCAG
CATCTCAGCACTTTCTTTCCACGTCTGTCCAGGGTCCCTGGGAGAGAGCCATCTCGCCAAACAAAGTGCCCTACTAT
ATCAACCACGAGACTCAAACAACTTGCTGGGACCATCCCAAAATGACAGAGCTCTACCAGTCTTTAGCTGACCTGAA
TAATGTCAGATTCTCAGCTTATAGGACTGCCATGAAACTCCGAAGACTGCAGAAGGCCCTTTGCTTGGATCTCTTGA
GCCTGTCAGCTGCATGTGATGCCTTGGACCAGCACAACCTCAAGCAAAATGACCAGCCCATGGATATCCTGCAGATT
ATTAATTGTTTGACCACTATTTATGACCGCCTGGAGCAAGAGCACAACAATTTGGTCAACGTCCCTCTCTGCGTGGA
TATGTGTCTGAACTGGCTGCTGAATGTTTATGATACGGGACGAACAGGGAGGATCCGTGTCCTGTCTTTTAAAACTG
GCATCATTTCCCTGTGTAAAGCACATTTGGAAGACAAGTACAGATACCTTTTCAAGCAAGTGGCAAGTTCAACAGGA
TTTTGTGACCAGCGCAGGCTGGGCCTCCTTCTGCATGATTCTATCCAAATTCCAAGACAGTTGGGTGAAGTTGCATC
CTTTGGGGGCAGTAACATTGAGCCAAGTGTCCGGAGCTGCTTCCAATTTGCTAATAATAAGCCAGAGATCGAAGCGG
CCCTCTTCCTAGACTGGATGAGACTGGAACCCCAGTCCATGGTGTGGCTGCCCGTCCTGCACAGAGTGGCTGCTGCA
GAAACTGCCAAGCATCAGGCCAAATGTAACATCTGCAAAGAGTGTCCAATCATTGGATTCAGGTACAGGAGTCTAAA
GCACTTTAATTATGACATCTGCCAAAGCTGCTTTTTTTCTGGTCGAGTTGCAAAAGGCCATAAAATGCACTATCCCA
TGGTGGAATATTGCACTCCGACTACATCAGGAGAAGATGTTCGAGACTTTGCCAAGGTACTAAAAAACAAATTTCGA
ACCAAAAGGTATTTTGCGAAGCATCCCCGAATGGGCTACCTGCCAGTGCAGACTGTCTTAGAGGGGGACAACATGGA
AACTCCCGTTACTCTGATCAACTTCTGGCCAGTAGATTCTGCGCCTGCCTCGTCCCCTCAGCTTTCACACGATGATA
CTCATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCCCC
CCTGAGCCAGCCTCGTAGTCCTGCCCAGATCTTGATTTCCTTAGAGAGTGAGGAAAGAGGGGAGCTAGAGAGAATCC
TAGCAGATCTTGAGGAAGAAAACAGGAATCTGCAAGCAGAATATGACCGTCTAAAGCAGCAGCACGAACATAAAGGC
CTGTCCCCACTGCCGTCCCCTCCTGAAATGATGCCCACCTCTCCCCAGAGTCCC
Nterm-R1_R17-R19_H4 to end of dystrophin:
(SEQ ID NO: 11)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTTGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGGGTCCAGCATCTCAGCACTTTCTTTCCACGTCTGTCCAGGGTCCCTGGGAGAG
AGCCATCTCGCCAAACAAAGTGCCCTACTATATCAACCACGAGACTCAAACAACTTGCTGGGACCATCCCAAAATGA
CAGAGCTCTACCAGTCTTTAGCTGACCTGAATAATGTCAGATTCTCAGCTTATAGGACTGCCATGAAACTCCGAAGA
CTGCAGAAGGCCCTTTGCTTGGATCTCTTGAGCCTGTCAGCTGCATGTGATGCCTTGGACCAGCACAACCTCAAGCA
AAATGACCAGCCCATGGATATCCTGCAGATTATTAATTGTTTGACCACTATTTATGACCGCCTGGAGCAAGAGCACA
ACAATTTGGTCAACGTCCCTCTCTGCGTGGATATGTGTCTGAACTGGCTGCTGAATGTTTATGATACGGGACGAACA
GGGAGGATCCGTGTCCTGTCTTTTAAAACTGGCATCATTTCCCTGTGTAAAGCACATTTGGAAGACAAGTACAGATA
CCTTTTCAAGCAAGTGGCAAGTTCAACAGGATTTTGTGACCAGCGCAGGCTGGGCCTCCTTCTGCATGATTCTATCC
AAATTCCAAGACAGTTGGGTGAAGTTGCATCCTTTGGGGGCAGTAACATTGAGCCAAGTGTCCGGAGCTGCTTCCAA
TTTGCTAATAATAAGCCAGAGATCGAAGCGGCCCTCTTCCTAGACTGGATGAGACTGGAACCCCAGTCCATGGTGTG
GCTGCCCGTCCTGCACAGAGTGGCTGCTGCAGAAACTGCCAAGCATCAGGCCAAATGTAACATCTGCAAAGAGTGTC
CAATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGAGA
CTTTGCCAAGGTACTAAAAAACAAATTTCGAACCAAAAGGTATTTTGCGAAGCATCCCCGAATGGGCTACCTGCCAG
TGCAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATGGA
AAACAGCAATGGATCTTATCTAAATGATAGCATCTCTCCTAATGAGAGCATAGATGATGAACATTTGTTAATCCAGC
ATTACTGCCAAAGTTTGAACCAGGACTCCCCCCTGAGCCAGCCTCGTAGTCCTGCCCAGATCTTGATTTCCTTAGAG
AGTGAGGAAAGAGGGGAGCTAGAGAGAATCCTAGCAGATCTTGAGGAAGAAAACAGGAATCTGCAAGCAGAATATGA
CCGTCTAAAGCAGCAGCACGAACATAAAGGCCTGTCCCCACTGCCGTCCCCTCCTGAAATGATGCCCACCTCTCCCC
AGAGTCCCCGGGATGCTGAGCTCATTGCTGAGGCCAAGCTACTGCGTCAACACAAAGGCCGCCTGGAAGCCAGGATG
CAAATCCTGGAAGACCACAATAAACAGCTGGAGTCACAGTTACACAGGCTAAGGCAGCTGCTGGAGCAACCCCAGGC
AGAGGCCAAAGTGAATGGCACAACGGTGTCCTCTCCTTCTACCTCTCTACAGAGGTCCGACAGCAGTCAGCCTATGC
TGCTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAGCC
AATGAGAGAGGACACAATG
Nterm-R1_R17-R19_H4 to end of dystrophin:
(SEQ ID NO: 96)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTIGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGGGTCCAGCATCTCAGCACTTTCTTTCCACGTCTGTCCAGGGTCCCTGGGAGAGAGCCATCTC
GCCAAACAAAGTGCCCTACTATATCAACCACGAGACTCAAACAACTTGCTGGGACCATCCCAAAATGACAGAGCTCT
ACCAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGACCA
GCCCATGGATATCCTGCAGATTATTAATTGTTTGACCACTATTTATGACCGCCTGGAGCAAGAGCACAACAATTTGG
TCAACGTCCCTCTCTGCGTGGATATGTGTCTGAACTGGCTGCTGAATGTTTATGATACGGGACGAACAGGGAGGATC
CGTGTCCTGTCTTTTAAAACTGGCATCATTTCCCTGTGTAAAGCACATTTGGAAGACAAGTACAGATACCTTTTCAA
GCAAGTGGCAAGTTCAACAGGATTTTGTGACCAGCGCAGGCTGGGCCTCCTTCTGCATGATTCTATCCAAATTCCAA
GACAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCCGT
CCTGCACAGAGTGGCTGCTGCAGAAACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGCTTTTTTTCTGGTCGAGTTGCAAAA
GGCCATAAAATGCACTATCCCATGGTGGAATATTGCACTCCGACTACATCAGGAGAAGATGTTCGAGACTTTGCCAA
GGTACTAAAAAACAAATTTCGAACCAAAAGGTATTTTGCGAAGCATCCCCGAATGGGCTACCTGCCAGTGCAGACTG
TCTTAGAGGGGGACAACATGGAAACTCCCGTTACTCTGATCAACTTCTGGCCAGTAGATTCTGCGCCTGCCTCGTCC
CCTCAGCTTTCACACGATGATACTCATTCACGCATTGAACATTATGCTAGCAGGCTAGCAGAAATGGAAAACAGCAA
TGGATCTTATCTAAATGATAGCATCTCTCCTAATGAGAGCATAGATGATGAACATTTGTTAATCCAGCATTACTGCC
AAAGTTTGAACCAGGACTCCCCCCTGAGCCAGCCTCGTAGTCCTGCCCAGATCTTGATTTCCTTAGAGAGTGAGGAA
AGAGGGGAGCTAGAGAGAATCCTAGCAGATCTTGAGGAAGAAAACAGGAATCTGCAAGCAGAATATGACCGTCTAAA
GCAGCAGCACGAACATAAAGGCCTGTCCCCACTGCCGTCCCCTCCTGAAATGATGCCCACCTCTCCCCAGAGTCCCC
GGGATGCTGAGCTCATTGCTGAGGCCAAGCTACTGCGTCAACACAAAGGCCGCCTGGAAGCCAGGATGCAAATCCTG
GAAGACCACAATAAACAGCTGGAGTCACAGTTACACAGGCTAAGGCAGCTGCTGGAGCAACCCCAGGCAGAGGCCAA
AGTGAATGGCACAACGGTGTCCTCTCCTTCTACCTCTCTACAGAGGTCCGACAGCAGTCAGCCTATGCTGCTCCGAG
TGGTTGGCAGTCAAACTTCGGACTCCATGGGTGAGGAAGATCTTCTCAGTCCTCCCCAGGACACAAGCACAGGGTTA
GAGGAGGTGATGGAGCAACTCAACAACTCCTTCCCTAGTTCAAGAGGAAGAAATACCCCTGGAAAGCCAATGAGAGA
GGACACAATG
Nterm-R1_R17-R19_R24 to end of dystrophin:
(SEQ ID NO: 12)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGAA
TATCTTGTCAGAATTTCAAAGAGATTTAAATGAATTTGTTTTATGGTTGGAGGAAGCAGATAACATTGCTAGTATCC
CACTTGAACCTGGAAAAGAGCAGCAACTAAAAGAAAAGCTTGAGCAAGTCAAGTTACTGGTGGAAGAGTTGCCCCTG
CGCCAGGGAATTCTCAAACAATTAAATGAAACTGGAGGACCCGTGCTTGTAAGTGCTCCCATAAGCCCAGAAGAGCA
AGATAAACTTGAAAATAAGCTCAAGCAGACAAATCTCCAGTGGATAAAGGTTTCCAGAGCTTTACCTGAGAAACAAG
GAGAAATTGAAGCTCAAATAAAAGACCTTGGGCAGCTTGAAAAAAAGCTTGAAGACCTTGAAGAGCAGTTAAATCAT
CTGCTGCTGTGGTTATCTCCTATTAGGAATCAGTTGGAAATTTATAACCAACCAAACCAAGAAGGACCATTIGACGT
TAAGGAAACTGAAATAGCAGTTCAAGCTAAACAACCGGATGTGGAAGAGATTTTGTCTAAAGGGCAGCATTTGTACA
AGGAAAAACCAGCCACTCAGCCAGTGAAGAGGAAGTTAGAAGATCTGAGCTCTGAGTGGAAGGCGGTAAACCGTTTA
CTTCAAGAGCTGAGGGCAAAGCAGTGGCAGAGAAAAATAGATGAGACCCTTGAAAGACTCCGGGAACTTCAAGAGGC
CACGGATGAGCTGGACCTCAAGCTGCGCCAAGCTGAGGTGATCAAGGGATCCTGGCAGCCCGTGGGCGATCTCCTCA
TTGACTCTCTCCAAGATCACCTCGAGAAAGTCAAGGCACTTCGAGGAGAAATTGCGCCTCTGAAAGAGAACGTGAGC
CACGTCAATGACCTTGCTCGCCAGCTTACCACTTTGGGCATTCAGCTCTCACCGTATAACCTCAGCACTCTGGAAGA
CCTGAACACCAGATGGAAGCTTCTGCAGGTGGCCGTCGAGGACCGAGTCAGGCAGCTGCATGAAGCCCACAGGGACT
TTGGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTAGC
TGACCTGAATAATGTCAGATTCTCAGCTTATAGGACTGCCATGAAACTCCGAAGACTGCAGAAGGCCCTTTGCTTGG
ATCTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCTCT
CTGCGTGGATATGTGTCTGAACTGGCTGCTGAATGTTTATGATACGGGACGAACAGGGAGGATCCGTGTCCTGTCTT
TTAAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGTGA
AGTTGCATCCTTTGGGGGCAGTAACATTGAGCCAAGTGTCCGGAGCTGCTTCCAATTTGCTAATAATAAGCCAGAGA
TCGAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTACAG
GAGTCTAAAGCACTTTAATTATGACATCTGCCAAAGCTGCTTTTTTTCTGGTCGAGTTGCAAAAGGCCATAAAATGC
ACTATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGGGA
CAACATGGAAACTCCCGTTACTCTGATCAACTTCTGGCCAGTAGATTCTGCGCCTGCCTCGTCCCCTCAGCTTTCAC
ACGATGATACTCATTCACGCATTGAACATTATGCTAGCAGGCTAGCAGAAATGGAAAACAGCAATGGATCTTATCTA
AATGATAGCATCTCTCCTAATGAGAGCATAGATGATGAACATTTGTTAATCCAGCATTACTGCCAAAGTTTGAACCA
GGACTCCCCCCTGAGCCAGCCTCGTAGTCCTGCCCAGATCTTGATTTCCTTAGAGAGTGAGGAAAGAGGGGAGCTAG
AGAGAATCCTAGCAGATCTTGAGGAAGAAAACAGGAATCTGCAAGCAGAATATGACCGTCTAAAGCAGCAGCACGAA
CATAAAGGCCTGTCCCCACTGCCGTCCCCTCCTGAAATGATGCCCACCTCTCCCCAGAGTCCCCGGGATGCTGAGCT
CATTGCTGAGGCCAAGCTACTGCGTCAACACAAAGGCCGCCTGGAAGCCAGGATGCAAATCCTGGAAGACCACAATA
AACAGCTGGAGTCACAGTTACACAGGCTAAGGCAGCTGCTGGAGCAACCCCAGGCAGAGGCCAAAGTGAATGGCACA
ACGGTGTCCTCTCCTTCTACCTCTCTACAGAGGTCCGACAGCAGTCAGCCTATGCTGCTCCGAGTGGTTGGCAGTCA
AACTTCGGACTCCATGGGTGAGGAAGATCTTCTCAGTCCTCCCCAGGACACAAGCACAGGGTTAGAGGAGGTGATGG
AGCAACTCAACAACTCCTTCCCTAGTTCAAGAGGAAGAAATACCCCTGGAAAGCCAATGAGAGAGGACACAATG
Nterm-R1_R17-R19_R24 to end of dystrophin:
(SEQ ID NO: 97)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGAATATCTTGTC
AGAATTTCAAAGAGATTTAAATGAATTTGTTTTATGGTTGGAGGAAGCAGATAACATTGCTAGTATCCCACTTGAAC
CTGGAAAAGAGCAGCAACTAAAAGAAAAGCTTGAGCAAGTCAAGTTACTGGTGGAAGAGTTGCCCCTGCGCCAGGGA
ATTCTCAAACAATTAAATGAAACTGGAGGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAGCTTTACCTGAGAAACAAGGAGAAATTG
AAGCTCAAATAAAAGACCTTGGGCAGCTTGAAAAAAAGCTTGAAGACCTTGAAGAGCAGTTAAATCATCTGCTGCTG
TGGTTATCTCCTATTAGGAATCAGTTGGAAATTTATAACCAACCAAACCAAGAAGGACCATTTGACGTTAAGGAAAC
TGAAATAGCAGTTCAAGCTAAACAACCGGATGTGGAAGAGATTTTGTCTAAAGGGCAGCATTTGTACAAGGAAAAAC
CAGCCACTCAGCCAGTGAAGAGGAAGTTAGAAGATCTGAGCTCTGAGTGGAAGGCGGTAAACCGTTTACTTCAAGAG
CTGAGGGCAAAGCAGTGGCAGAGAAAAATAGATGAGACCCTTGAAAGACTCCGGGAACTTCAAGAGGCCACGGATGA
GCTGGACCTCAAGCTGCGCCAAGCTGAGGTGATCAAGGGATCCTGGCAGCCCGTGGGCGATCTCCTCATTGACTCTC
TCCAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAACAC
CAGATGGAAGCTTCTGCAGGTGGCCGTCGAGGACCGAGTCAGGCAGCTGCATGAAGCCCACAGGGACTTTGGTCCAG
CATCTCAGCACTTTCTTTCCACGTCTGTCCAGGGTCCCTGGGAGAGAGCCATCTCGCCAAACAAAGTGCCCTACTAT
ATCAACCACGAGACTCAAACAACTTGCTGGGACCATCCCAAAATGACAGAGCTCTACCAGTCTTTAGCTGACCTGAA
TAATGTCAGATTCTCAGCTTATAGGACTGCCATGAAACTCCGAAGACTGCAGAAGGCCCTTTGCTTGGATCTCTTGA
GCCTGTCAGCTGCATGTGATGCCTTGGACCAGCACAACCTCAAGCAAAATGACCAGCCCATGGATATCCTGCAGATT
ATTAATTGTTTGACCACTATTTATGACCGCCTGGAGCAAGAGCACAACAATTTGGTCAACGTCCCTCTCTGCGTGGA
TATGTGTCTGAACTGGCTGCTGAATGTTTATGATACGGGACGAACAGGGAGGATCCGTGTCCTGTCTTTTAAAACTG
GCATCATTTCCCTGTGTAAAGCACATTTGGAAGACAAGTACAGATACCTTTTCAAGCAAGTGGCAAGTTCAACAGGA
TTTTGTGACCAGCGCAGGCTGGGCCTCCTTCTGCATGATTCTATCCAAATTCCAAGACAGTTGGGTGAAGTTGCATC
CTTTGGGGGCAGTAACATTGAGCCAAGTGTCCGGAGCTGCTTCCAATTTGCTAATAATAAGCCAGAGATCGAAGCGG
CCCTCTTCCTAGACTGGATGAGACTGGAACCCCAGTCCATGGTGTGGCTGCCCGTCCTGCACAGAGTGGCTGCTGCA
GAAACTGCCAAGCATCAGGCCAAATGTAACATCTGCAAAGAGTGTCCAATCATTGGATTCAGGTACAGGAGTCTAAA
GCACTTTAATTATGACATCTGCCAAAGCTGCTTTTTTTCTGGTCGAGTTGCAAAAGGCCATAAAATGCACTATCCCA
TGGTGGAATATTGCACTCCGACTACATCAGGAGAAGATGTTCGAGACTTTGCCAAGGTACTAAAAAACAAATTTCGA
ACCAAAAGGTATTTTGCGAAGCATCCCCGAATGGGCTACCTGCCAGTGCAGACTGTCTTAGAGGGGGACAACATGGA
AACTCCCGTTACTCTGATCAACTTCTGGCCAGTAGATTCTGCGCCTGCCTCGTCCCCTCAGCTTTCACACGATGATA
CTCATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCCCC
CCTGAGCCAGCCTCGTAGTCCTGCCCAGATCTTGATTTCCTTAGAGAGTGAGGAAAGAGGGGAGCTAGAGAGAATCC
TAGCAGATCTTGAGGAAGAAAACAGGAATCTGCAAGCAGAATATGACCGTCTAAAGCAGCAGCACGAACATAAAGGC
CTGTCCCCACTGCCGTCCCCTCCTGAAATGATGCCCACCTCTCCCCAGAGTCCCCGGGATGCTGAGCTCATTGCTGA
GGCCAAGCTACTGCGTCAACACAAAGGCCGCCTGGAAGCCAGGATGCAAATCCTGGAAGACCACAATAAACAGCTGG
AGTCACAGTTACACAGGCTAAGGCAGCTGCTGGAGCAACCCCAGGCAGAGGCCAAAGTGAATGGCACAACGGTGTCC
TCTCCTTCTACCTCTCTACAGAGGTCCGACAGCAGTCAGCCTATGCTGCTCCGAGTGGTTGGCAGTCAAACTTCGGA
CTCCATGGGTGAGGAAGATCTTCTCAGTCCTCCCCAGGACACAAGCACAGGGTTAGAGGAGGTGATGGAGCAACTCA
ACAACTCCTTCCCTAGTTCAAGAGGAAGAAATACCCCTGGAAAGCCAATGAGAGAGGACACAATG
Nterm-R1_R17_H3_R21-R22_H4 to end of syntrophin region
(SEQ ID NO: 109)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGCA
GCCTGACCTAGCTCCTGGACTGACCACTATTGGAGCCTCTCCTACTCAGACTGTTACTCTGGTGACACAACCTGTGG
TTACTAAGGAAACTGCCATCTCCAAACTAGAAATGCCATCTTCCTTGATGTTGGAGGTACCTAAGGATTCAACACAA
TGGCTGGAAGCTAAGGAAGAAGCTGAGCAGGTCTTAGGACAGGCCAGAGCCAAGCTTGAGTCATGGAAGGAGGGTCC
CTATACAGTAGATGCAATCCAAAAGAAAATCACAGAAACCAAGCAGTTGGCCAAAGACCTCCGCCAGTGGCAGACAA
ATGTAGATGTGGCAAATGACTTGGCCCTGAAACTTCTCCGGGATTATTCTGCAGATGATACCAGAAAAGTCCACATG
ATAACAGAGAATATCAATGCCTCTTGGAGAAGCATTCATAAAAGGGTGAGTGAGCGAGAGGCTGCTTTGGAAGAAAC
TCATAGATTACTGCAACAGTTCCCCCTGGACCTGGAAAAGTTTCTTGCCTGGCTTACAGAAGCTGAAACAACTGCCA
ATGTCCTACAGGATGCTACCCGTAAGGAAAGGCTCCTAGAAGACTCCAAGGGAGTAAAAGAGCTGATGAAACAATGG
CAAGACCTCCAAGGTGAAATTGAAGCTCACACAGATGTTTATCACAACCTGGATGAAAACAGCCAAAAAATCCTGAG
ATCCCTGGAAGGTTCCGATGATGCAGTCCTGTTACAAAGACGTTTGGATAACATGAACTTCAAGTGGAGTGAACTTC
GGAAAAAGTCTCTCAACATTAGGTCCCATTTGGAAGCCAGTTCTGACCAGTGGAAGCGTCTGGGTCCAGCATCTCAG
CACTTTCTTTCCACGTCTGTCCAGGGTCCCTGGGAGAGAGCCATCTCGCCAAACAAAGTGCCCTACTATATCAACCA
CGAGACTCAAACAACTTGCTGGGACCATCCCAAAATGACAGAGCTCTACCAGTCTTTAGCTGACCTGAATAATGTCA
GATTCTCAGCTTATAGGACTGCCATGAAACTCCGAAGACTGCAGAAGGCCCTTTGCTTGGATCTCTTGAGCCTGTCA
GCTGCATGTGATGCCTTGGACCAGCACAACCTCAAGCAAAATGACCAGCCCATGGATATCCTGCAGATTATTAATTG
TTTGACCACTATTTATGACCGCCTGGAGCAAGAGCACAACAATTTGGTCAACGTCCCTCTCTGCGTGGATATGTGTC
TGAACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTGTGA
CCAGCGCAGGCTGGGCCTCCTTCTGCATGATTCTATCCAAATTCCAAGACAGTTGGGTGAAGTTGCATCCTTTGGGG
GCAGTAACATTGAGCCAAGTGTCCGGAGCTGCTTCCAATTTGCTAATAATAAGCCAGAGATCGAAGCGGCCCTCTTC
CTAGACTGGATGAGACTGGAACCCCAGTCCATGGTGTGGCTGCCCGTCCTGCACAGAGTGGCTGCTGCAGAAACTGC
CAAGCATCAGGCCAAATGTAACATCTGCAAAGAGTGTCCAATCATTGGATTCAGGTACAGGAGTCTAAAGCACTTTA
ATTATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAAAG
GTATTTTGCGAAGCATCCCCGAATGGGCTACCTGCCAGTGCAGACTGTCTTAGAGGGGGACAACATGGAAACTCCCG
TTACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCTCC
TAATGAGAGCATAGATGATGAACATTTGTTAATCCAGCATTACTGCCAAAGTTTGAACCAGGACTCCCCCCTGAGCC
AGCCTCGTAGT
Nterm-R1_R17_H3_R21-R22_H4 to end of syntrophin region
Codon optimized sequence
(SEQ ID NO: 110)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCGGTTCGACCGGAGCGTGGAGAAGTGGCGGCGG
TTCCACTACGACATCAAGATCTTCAACCAGTGGCTGACCGAGGCCGAGCAGTTCCTGCGGAAGACCCAGATCCCCGA
GAACTGGGAGCACGCCAAGTACAAGTGGTACCTGAAGGAGCTGCAGGACGGCATCGGCCAGCGGCAGACCGTGGTGC
GGACCCTGAACGCCACCGGCGAGGAGATCATCCAGCAGAGCAGCAAGACCGACGCCAGCATCCTGCAGGAGAAGCTG
GGCAGCCTGAACCTGCGGTGGCAGGAGGTGTGCAAGCAGCTGAGCGACCGGAAGAAGCGGCTGGAGGAGCAGAAGCA
GCCCGACCTGGCCCCCGGCCTGACCACCATCGGCGCCAGCCCCACCCAGACCGTGACCCTGGTGACCCAGCCCGTGG
TGACCAAGGAGACCGCCATCAGCAAGCTGGAGATGCCCAGCAGCCTGATGCTGGAGGTGCCCAAGGACAGCACCCAG
TGGCTGGAGGCCAAGGAGGAGGCCGAGCAGGTGCTGGGCCAGGCCCGGGCCAAGCTGGAGAGCTGGAAGGAGGGCCC
CTACACCGTGGACGCCATCCAGAAGAAGATCACCGAGACCAAGCAGCTGGCCAAGGACCTGCGGCAGTGGCAGACCA
ACGTGGACGTGGCCAACGACCTGGCCCTGAAGCTGCTGCGGGACTACAGCGCCGACGACACCCGGAAGGTGCACATG
ATCACCGAGAACATCAACGCCAGCTGGCGGAGCATCCACAAGCGGGTGAGCGAGCGGGAGGCCGCCCTGGAGGAGAC
CCACCGGCTGCTGCAGCAGTTCCCCCTGGACCTGGAGAAGTTCCTGGCCTGGCTGACCGAGGCCGAGACCACCGCCA
ACGTGCTGCAGGACGCCACCCGGAAGGAGCGGCTGCTGGAGGACAGCAAGGGCGTGAAGGAGCTGATGAAGCAGTGG
CAGGACCTGCAGGGCGAGATCGAGGCCCACACCGACGTGTACCACAACCTGGACGAGAACAGCCAGAAGATCCTGCG
GAGCCTGGAGGGCAGCGACGACGCCGTGCTGCTGCAGCGGCGGCTGGACAACATGAACTTCAAGTGGAGCGAGCTGC
GGAAGAAGAGCCTGAACATCCGGAGCCACCTGGAGGCCAGCAGCGACCAGTGGAAGCGGCTGGGCCCCGCCAGCCAG
CACTTCCTGAGCACCAGCGTGCAGGGCCCCTGGGAGCGGGCCATCAGCCCCAACAAGGTGCCCTACTACATCAACCA
CGAGACCCAGACCACCTGCTGGGACCACCCCAAGATGACCGAGCTGTACCAGAGCCTGGCCGACCTGAACAACGTGC
GGTTCAGCGCCTACCGGACCGCCATGAAGCTGCGGCGGCTGCAGAAGGCCCTGTGCCTGGACCTGCTGAGCCTGAGC
GCCGCCTGCGACGCCCTGGACCAGCACAACCTGAAGCAGAACGACCAGCCCATGGACATCCTGCAGATCATCAACTG
CCTGACCACCATCTACGACCGGCTGGAGCAGGAGCACAACAACCTGGTGAACGTGCCCCTGTGCGTGGACATGTGCC
TGAACTGGCTGCTGAACGTGTACGACACCGGCCGGACCGGCCGGATCCGGGTGCTGAGCTTCAAGACCGGCATCATC
AGCCTGTGCAAGGCCCACCTGGAGGACAAGTACCGGTACCTGTTCAAGCAGGTGGCCAGCAGCACCGGCTTCTGCGA
CCAGCGGCGGCTGGGCCTGCTGCTGCACGACAGCATCCAGATCCCCCGGCAGCTGGGCGAGGTGGCCAGCTTCGGCG
GCAGCAACATCGAGCCCAGCGTGCGGAGCTGCTTCCAGTTCGCCAACAACAAGCCCGAGATCGAGGCCGCCCTGTTC
CTGGACTGGATGCGGCTGGAGCCCCAGAGCATGGTGTGGCTGCCCGTGCTGCACCGGGTGGCCGCCGCCGAGACCGC
CAAGCACCAGGCCAAGTGCAACATCTGCAAGGAGTGCCCCATCATCGGCTTCCGGTACCGGAGCCTGAAGCACTTCA
ACTACGACATCTGCCAGAGCTGCTTCTTCAGCGGCCGGGTGGCCAAGGGCCACAAGATGCACTACCCCATGGTGGAG
TACTGCACCCCCACCACCAGCGGCGAGGACGTGCGGGACTTCGCCAAGGTGCTGAAGAACAAGTTCCGGACCAAGCG
GTACTTCGCCAAGCACCCCCGGATGGGCTACCTGCCCGTGCAGACCGTGCTGGAGGGCGACAACATGGAGACCCCCG
TGACCCTGATCAACTTCTGGCCCGTGGACAGCGCCCCCGCCAGCAGCCCCCAGCTGAGCCACGACGACACCCACAGC
CGGATCGAGCACTACGCCAGCCGGCTGGCCGAGATGGAGAACAGCAACGGCAGCTACCTGAACGACAGCATCAGCCC
CAACGAGAGCATCGACGACGAGCACCTGCTGATCCAGCACTACTGCCAGAGCCTGAACCAGGACAGCCCCCTGAGCC
AGCCCCGGAGC
Nterm-R1_R17_R22_H4 to 1st coiled coil
(SEQ ID NO: 111)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCGATTTGACAGATCTGTTGAGAAATGGCGGCGT
TTTCATTATGATATAAAGATATTTAATCAGTGGCTAACAGAAGCTGAACAGTTTCTCAGAAAGACACAAATTCCTGA
GAATTGGGAACATGCTAAATACAAATGGTATCTTAAGGAACTCCAGGATGGCATTGGGCAGCGGCAAACTGTTGTCA
GAACATTGAATGCAACTGGGGAAGAAATAATTCAGCAATCCTCAAAAACAGATGCCAGTATTCTACAGGAAAAATTG
GGAAGCCTGAATCTGCGGTGGCAGGAGGTCTGCAAACAGCTGTCAGACAGAAAAAAGAGGCTAGAAGAACAAAAGGA
AGAAACTCATAGATTACTGCAACAGTTCCCCCTGGACCTGGAAAAGTTTCTTGCCTGGCTTACAGAAGCTGAAACAA
CTGCCAATGTCCTACAGGATGCTACCCGTAAGGAAAGGCTCCTAGAAGACTCCAAGGGAGTAAAAGAGCTGATGAAA
CAATGGCAAGACCTCCAAGGTGAAATTGAAGCTCACACAGATGTTTATCACAACCTGGATGAAAACAGCCAAAAAAT
CCTGAGATCCCTGGAAGGTTCCGATGATGCAGTCCTGTTACAAAGACGTTTGGATAACATGAACTTCAAGTGGAGTG
AACTTCGGAAAAAGTCTCTCAACATTAGGTCCCATTTGGAAGCCAGTTCTGACCAGTGGAAGCGTCTGGGTCCAGCA
TCTCAGCACTTTCTTTCCACGTCTGTCCAGGGTCCCTGGGAGAGAGCCATCTCGCCAAACAAAGTGCCCTACTATAT
CAACCACGAGACTCAAACAACTTGCTGGGACCATCCCAAAATGACAGAGCTCTACCAGTCTTTAGCTGACCTGAATA
ATGTCAGATTCTCAGCTTATAGGACTGCCATGAAACTCCGAAGACTGCAGAAGGCCCTTTGCTTGGATCTCTTGAGC
CTGTCAGCTGCATGTGATGCCTTGGACCAGCACAACCTCAAGCAAAATGACCAGCCCATGGATATCCTGCAGATTAT
TAATTGTTTGACCACTATTTATGACCGCCTGGAGCAAGAGCACAACAATTTGGTCAACGTCCCTCTCTGCGTGGATA
TGTGTCTGAACTGGCTGCTGAATGTTTATGATACGGGACGAACAGGGAGGATCCGTGTCCTGTCTTTTAAAACTGGC
ATCATTTCCCTGTGTAAAGCACATTTGGAAGACAAGTACAGATACCTTTTCAAGCAAGTGGCAAGTTCAACAGGATT
TTGTGACCAGCGCAGGCTGGGCCTCCTTCTGCATGATTCTATCCAAATTCCAAGACAGTTGGGTGAAGTTGCATCCT
TTGGGGGCAGTAACATTGAGCCAAGTGTCCGGAGCTGCTTCCAATTTGCTAATAATAAGCCAGAGATCGAAGCGGCC
CTCTTCCTAGACTGGATGAGACTGGAACCCCAGTCCATGGTGTGGCTGCCCGTCCTGCACAGAGTGGCTGCTGCAGA
AACTGCCAAGCATCAGGCCAAATGTAACATCTGCAAAGAGTGTCCAATCATTGGATTCAGGTACAGGAGTCTAAAGC
ACTTTAATTATGACATCTGCCAAAGCTGCTTTTTTTCTGGTCGAGTTGCAAAAGGCCATAAAATGCACTATCCCATG
GTGGAATATTGCACTCCGACTACATCAGGAGAAGATGTTCGAGACTTTGCCAAGGTACTAAAAAACAAATTTCGAAC
CAAAAGGTATTTTGCGAAGCATCCCCGAATGGGCTACCTGCCAGTGCAGACTGTCTTAGAGGGGGACAACATGGAAA
CTCCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGCAT
CTCTCCTAATGAGAGCATAGATGATGAACATTTGTTAATCCAGCATTACTGCCAAAGTTTGAACCAGGACTCCCCCC
TGAGCCAGCCTCGTAGTCCTGCCCAGATCTTGATTTCCTTAGAGAGTGAGGAAAGAGGGGAGCTAGAGAGAATCCTA
GCAGATCTTGAGGAAGAAAACAGGAATCTGCAAGCAGAATATGACCGTCTAAAGCAGCAGCACGAACATAAAGGCCT
GTCC
Nterm-R1_R17_R22_H4 to 1st coiled coil
Codon optimized sequence
(SEQ ID NO: 112)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCGGTTCGACCGGAGCGTGGAGAAGTGGCGGCGG
TTCCACTACGACATCAAGATCTTCAACCAGTGGCTGACCGAGGCCGAGCAGTTCCTGCGGAAGACCCAGATCCCCGA
GAACTGGGAGCACGCCAAGTACAAGTGGTACCTGAAGGAGCTGCAGGACGGCATCGGCCAGCGGCAGACCGTGGTGC
GGACCCTGAACGCCACCGGCGAGGAGATCATCCAGCAGAGCAGCAAGACCGACGCCAGCATCCTGCAGGAGAAGCTG
GGCAGCCTGAACCTGCGGTGGCAGGAGGTGTGCAAGCAGCTGAGCGACCGGAAGAAGCGGCTGGAGGAGCAGAAGGA
GGAGACCCACCGGCTGCTGCAGCAGTTCCCCCTGGACCTGGAGAAGTTCCTGGCCTGGCTGACCGAGGCCGAGACCA
CCGCCAACGTGCTGCAGGACGCCACCCGGAAGGAGCGGCTGCTGGAGGACAGCAAGGGCGTGAAGGAGCTGATGAAG
CAGTGGCAGGACCTGCAGGGCGAGATCGAGGCCCACACCGACGTGTACCACAACCTGGACGAGAACAGCCAGAAGAT
CCTGCGGAGCCTGGAGGGCAGCGACGACGCCGTGCTGCTGCAGCGGCGGCTGGACAACATGAACTTCAAGTGGAGCG
AGCTGCGGAAGAAGAGCCTGAACATCCGGAGCCACCTGGAGGCCAGCAGCGACCAGTGGAAGCGGCTGGGCCCCGCC
AGCCAGCACTTCCTGAGCACCAGCGTGCAGGGCCCCTGGGAGCGGGCCATCAGCCCCAACAAGGTGCCCTACTACAT
CAACCACGAGACCCAGACCACCTGCTGGGACCACCCCAAGATGACCGAGCTGTACCAGAGCCTGGCCGACCTGAACA
ACGTGCGGTTCAGCGCCTACCGGACCGCCATGAAGCTGCGGCGGCTGCAGAAGGCCCTGTGCCTGGACCTGCTGAGC
CTGAGCGCCGCCTGCGACGCCCTGGACCAGCACAACCTGAAGCAGAACGACCAGCCCATGGACATCCTGCAGATCAT
CAACTGCCTGACCACCATCTACGACCGGCTGGAGCAGGAGCACAACAACCTGGTGAACGTGCCCCTGTGCGTGGACA
TGTGCCTGAACTGGCTGCTGAACGTGTACGACACCGGCCGGACCGGCCGGATCCGGGTGCTGAGCTTCAAGACCGGC
ATCATCAGCCTGTGCAAGGCCCACCTGGAGGACAAGTACCGGTACCTGTTCAAGCAGGTGGCCAGCAGCACCGGCTT
CTGCGACCAGCGGCGGCTGGGCCTGCTGCTGCACGACAGCATCCAGATCCCCCGGCAGCTGGGCGAGGTGGCCAGCT
TCGGCGGCAGCAACATCGAGCCCAGCGTGCGGAGCTGCTTCCAGTTCGCCAACAACAAGCCCGAGATCGAGGCCGCC
CTGTTCCTGGACTGGATGCGGCTGGAGCCCCAGAGCATGGTGTGGCTGCCCGTGCTGCACCGGGTGGCCGCCGCCGA
GACCGCCAAGCACCAGGCCAAGTGCAACATCTGCAAGGAGTGCCCCATCATCGGCTTCCGGTACCGGAGCCTGAAGC
ACTTCAACTACGACATCTGCCAGAGCTGCTTCTTCAGCGGCCGGGTGGCCAAGGGCCACAAGATGCACTACCCCATG
GTGGAGTACTGCACCCCCACCACCAGCGGCGAGGACGTGCGGGACTTCGCCAAGGTGCTGAAGAACAAGTTCCGGAC
CAAGCGGTACTTCGCCAAGCACCCCCGGATGGGCTACCTGCCCGTGCAGACCGTGCTGGAGGGCGACAACATGGAGA
CCCCCGTGACCCTGATCAACTTCTGGCCCGTGGACAGCGCCCCCGCCAGCAGCCCCCAGCTGAGCCACGACGACACC
CACAGCCGGATCGAGCACTACGCCAGCCGGCTGGCCGAGATGGAGAACAGCAACGGCAGCTACCTGAACGACAGCAT
CAGCCCCAACGAGAGCATCGACGACGAGCACCTGCTGATCCAGCACTACTGCCAGAGCCTGAACCAGGACAGCCCCC
TGAGCCAGCCCCGGAGCCCCGCCCAGATCCTGATCAGCCTGGAGAGCGAGGAGCGGGGCGAGCTGGAGCGGATCCTG
GCCGACCTGGAGGAGGAGAACCGGAACCTGCAGGCCGAGTACGACCGGCTGAAGCAGCAGCACGAGCACAAGGGCCT
GAGC
Nterm-R1_R16-R17_R21-R22_H4 to end of syntrophin region
(SEQ ID NO: 113)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCACACTGTCCGTGAAGAAACGATGATGGTGATG
ACTGAAGACATGCCTTTGGAAATTTCTTATGTGCCTTCTACTTATTTGACTGAAATCACTCATGTCTCACAAGCCCT
ATTAGAAGTGGAACAACTTCTCAATGCTCCTGACCTCTGTGCTAAGGACTTTGAAGATCTCTTTAAGCAAGAGGAGT
CTCTGAAGAATATAAAAGATAGTCTACAACAAAGCTCAGGTCGGATTGACATTATTCATAGCAAGAAGACAGCAGCA
TTGCAAAGTGCAACGCCTGTGGAAAGGGTGAAGCTACAGGAAGCTCTCTCCCAGCTTGATTTCCAATGGGAAAAAGT
TAACAAAATGTACAAGGACCGACAAGGGCGATTTGACAGATCTGTTGAGAAATGGCGGCGTTTTCATTATGATATAA
AGATATTTAATCAGTGGCTAACAGAAGCTGAACAGTTTCTCAGAAAGACACAAATTCCTGAGAATTGGGAACATGCT
AAATACAAATGGTATCTTAAGGAACTCCAGGATGGCATTGGGCAGCGGCAAACTGTTGTCAGAACATTGAATGCAAC
TGGGGAAGAAATAATTCAGCAATCCTCAAAAACAGATGCCAGTATTCTACAGGAAAAATTGGGAAGCCTGAATCTGC
GGTGGCAGGAGGTCTGCAAACAGCTGTCAGACAGAAAAAAGAGGCTAGAAGAACAAAAGAAGGATTCAACACAATGG
CTGGAAGCTAAGGAAGAAGCTGAGCAGGTCTTAGGACAGGCCAGAGCCAAGCTTGAGTCATGGAAGGAGGGTCCCTA
TACAGTAGATGCAATCCAAAAGAAAATCACAGAAACCAAGCAGTTGGCCAAAGACCTCCGCCAGTGGCAGACAAATG
TAGATGTGGCAAATGACTTGGCCCTGAAACTTCTCCGGGATTATTCTGCAGATGATACCAGAAAAGTCCACATGATA
ACAGAGAATATCAATGCCTCTTGGAGAAGCATTCATAAAAGGGTGAGTGAGCGAGAGGCTGCTTTGGAAGAAACTCA
TAGATTACTGCAACAGTTCCCCCTGGACCTGGAAAAGTTTCTTGCCTGGCTTACAGAAGCTGAAACAACTGCCAATG
TCCTACAGGATGCTACCCGTAAGGAAAGGCTCCTAGAAGACTCCAAGGGAGTAAAAGAGCTGATGAAACAATGGCAA
GACCTCCAAGGTGAAATTGAAGCTCACACAGATGTTTATCACAACCTGGATGAAAACAGCCAAAAAATCCTGAGATC
CCTGGAAGGTTCCGATGATGCAGTCCTGTTACAAAGACGTTTGGATAACATGAACTTCAAGTGGAGTGAACTTCGGA
AAAAGTCTCTCAACATTAGGTCCCATTTGGAAGCCAGTTCTGACCAGTGGAAGCGTCTGGGTCCAGCATCTCAGCAC
TTTCTTTCCACGTCTGTCCAGGGTCCCTGGGAGAGAGCCATCTCGCCAAACAAAGTGCCCTACTATATCAACCACGA
GACTCAAACAACTTGCTGGGACCATCCCAAAATGACAGAGCTCTACCAGTCTTTAGCTGACCTGAATAATGTCAGAT
TCTCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGTTT
GACCACTATTTATGACCGCCTGGAGCAAGAGCACAACAATTTGGTCAACGTCCCTCTCTGCGTGGATATGTGTCTGA
ACTGGCTGCTGAATGTTTATGATACGGGACGAACAGGGAGGATCCGTGTCCTGTCTTTTAAAACTGGCATCATTTCC
CTGTGTAAAGCACATTTGGAAGACAAGTACAGATACCTTTTCAAGCAAGTGGCAAGTTCAACAGGATTTTGTGACCA
GCGCAGGCTGGGCCTCCTTCTGCATGATTCTATCCAAATTCCAAGACAGTTGGGTGAAGTTGCATCCTTTGGGGGCA
GTAACATTGAGCCAAGTGTCCGGAGCTGCTTCCAATTTGCTAATAATAAGCCAGAGATCGAAGCGGCCCTCTTCCTA
GACTGGATGAGACTGGAACCCCAGTCCATGGTGTGGCTGCCCGTCCTGCACAGAGTGGCTGCTGCAGAAACTGCCAA
GCATCAGGCCAAATGTAACATCTGCAAAGAGTGTCCAATCATTGGATTCAGGTACAGGAGTCTAAAGCACTTTAATT
ATGACATCTGCCAAAGCTGCTTTTTTTCTGGTCGAGTTGCAAAAGGCCATAAAATGCACTATCCCATGGTGGAATAT
TGCACTCCGACTACATCAGGAGAAGATGTTCGAGACTTTGCCAAGGTACTAAAAAACAAATTTCGAACCAAAAGGTA
TTTTGCGAAGCATCCCCGAATGGGCTACCTGCCAGTGCAGACTGTCTTAGAGGGGGACAACATGGAAACTCCCGTTA
CTCTGATCAACTTCTGGCCAGTAGATTCTGCGCCTGCCTCGTCCCCTCAGCTTTCACACGATGATACTCATTCACGC
ATTGAACATTATGCTAGCAGGCTAGCAGAAATGGAAAACAGCAATGGATCTTATCTAAATGATAGCATCTCTCCTAA
TGAGAGCATAGATGATGAACATTTGTTAATCCAGCATTACTGCCAAAGTTTGAACCAGGACTCCCCCCTGAGCCAGC
CTCGTAGT
Nterm-R1_R16-R17_R21-R22_H4 to end of syntrophin region
Codon optimized sequence
(SEQ ID NO: 114)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCACACCGTGCGGGAGGAGACCATGATGGTGATG
ACCGAGGACATGCCCCTGGAGATCAGCTACGTGCCCAGCACCTACCTGACCGAGATCACCCACGTGAGCCAGGCCCT
GCTGGAGGTGGAGCAGCTGCTGAACGCCCCCGACCTGTGCGCCAAGGACTTCGAGGACCTGTTCAAGCAGGAGGAGA
GCCTGAAGAACATCAAGGACAGCCTGCAGCAGAGCAGCGGCCGGATCGACATCATCCACAGCAAGAAGACCGCCGCC
CTGCAGAGCGCCACCCCCGTGGAGCGGGTGAAGCTGCAGGAGGCCCTGAGCCAGCTGGACTTCCAGTGGGAGAAGGT
GAACAAGATGTACAAGGACCGGCAGGGCCGGTTCGACCGGAGCGTGGAGAAGTGGCGGCGGTTCCACTACGACATCA
AGATCTTCAACCAGTGGCTGACCGAGGCCGAGCAGTTCCTGCGGAAGACCCAGATCCCCGAGAACTGGGAGCACGCC
AAGTACAAGTGGTACCTGAAGGAGCTGCAGGACGGCATCGGCCAGCGGCAGACCGTGGTGCGGACCCTGAACGCCAC
CGGCGAGGAGATCATCCAGCAGAGCAGCAAGACCGACGCCAGCATCCTGCAGGAGAAGCTGGGCAGCCTGAACCTGC
GGTGGCAGGAGGTGTGCAAGCAGCTGAGCGACCGGAAGAAGCGGCTGGAGGAGCAGAAGAAGGACAGCACCCAGTGG
CTGGAGGCCAAGGAGGAGGCCGAGCAGGTGCTGGGCCAGGCCCGGGCCAAGCTGGAGAGCTGGAAGGAGGGCCCCTA
CACCGTGGACGCCATCCAGAAGAAGATCACCGAGACCAAGCAGCTGGCCAAGGACCTGCGGCAGTGGCAGACCAACG
TGGACGTGGCCAACGACCTGGCCCTGAAGCTGCTGCGGGACTACAGCGCCGACGACACCCGGAAGGTGCACATGATC
ACCGAGAACATCAACGCCAGCTGGCGGAGCATCCACAAGCGGGTGAGCGAGCGGGAGGCCGCCCTGGAGGAGACCCA
CCGGCTGCTGCAGCAGTTCCCCCTGGACCTGGAGAAGTTCCTGGCCTGGCTGACCGAGGCCGAGACCACCGCCAACG
TGCTGCAGGACGCCACCCGGAAGGAGCGGCTGCTGGAGGACAGCAAGGGCGTGAAGGAGCTGATGAAGCAGTGGCAG
GACCTGCAGGGCGAGATCGAGGCCCACACCGACGTGTACCACAACCTGGACGAGAACAGCCAGAAGATCCTGCGGAG
CCTGGAGGGCAGCGACGACGCCGTGCTGCTGCAGCGGCGGCTGGACAACATGAACTTCAAGTGGAGCGAGCTGCGGA
AGAAGAGCCTGAACATCCGGAGCCACCTGGAGGCCAGCAGCGACCAGTGGAAGCGGCTGGGCCCCGCCAGCCAGCAC
TTCCTGAGCACCAGCGTGCAGGGCCCCTGGGAGCGGGCCATCAGCCCCAACAAGGTGCCCTACTACATCAACCACGA
GACCCAGACCACCTGCTGGGACCACCCCAAGATGACCGAGCTGTACCAGAGCCTGGCCGACCTGAACAACGTGCGGT
TCAGCGCCTACCGGACCGCCATGAAGCTGCGGCGGCTGCAGAAGGCCCTGTGCCTGGACCTGCTGAGCCTGAGCGCC
GCCTGCGACGCCCTGGACCAGCACAACCTGAAGCAGAACGACCAGCCCATGGACATCCTGCAGATCATCAACTGCCT
GACCACCATCTACGACCGGCTGGAGCAGGAGCACAACAACCTGGTGAACGTGCCCCTGTGCGTGGACATGTGCCTGA
ACTGGCTGCTGAACGTGTACGACACCGGCCGGACCGGCCGGATCCGGGTGCTGAGCTTCAAGACCGGCATCATCAGC
CTGTGCAAGGCCCACCTGGAGGACAAGTACCGGTACCTGTTCAAGCAGGTGGCCAGCAGCACCGGCTTCTGCGACCA
GCGGCGGCTGGGCCTGCTGCTGCACGACAGCATCCAGATCCCCCGGCAGCTGGGCGAGGTGGCCAGCTTCGGCGGCA
GCAACATCGAGCCCAGCGTGCGGAGCTGCTTCCAGTTCGCCAACAACAAGCCCGAGATCGAGGCCGCCCTGTTCCTG
GACTGGATGCGGCTGGAGCCCCAGAGCATGGTGTGGCTGCCCGTGCTGCACCGGGTGGCCGCCGCCGAGACCGCCAA
GCACCAGGCCAAGTGCAACATCTGCAAGGAGTGCCCCATCATCGGCTTCCGGTACCGGAGCCTGAAGCACTTCAACT
ACGACATCTGCCAGAGCTGCTTCTTCAGCGGCCGGGTGGCCAAGGGCCACAAGATGCACTACCCCATGGTGGAGTAC
TGCACCCCCACCACCAGCGGCGAGGACGTGCGGGACTTCGCCAAGGTGCTGAAGAACAAGTTCCGGACCAAGCGGTA
CTTCGCCAAGCACCCCCGGATGGGCTACCTGCCCGTGCAGACCGTGCTGGAGGGCGACAACATGGAGACCCCCGTGA
CCCTGATCAACTTCTGGCCCGTGGACAGCGCCCCCGCCAGCAGCCCCCAGCTGAGCCACGACGACACCCACAGCCGG
ATCGAGCACTACGCCAGCCGGCTGGCCGAGATGGAGAACAGCAACGGCAGCTACCTGAACGACAGCATCAGCCCCAA
CGAGAGCATCGACGACGAGCACCTGCTGATCCAGCACTACTGCCAGAGCCTGAACCAGGACAGCCCCCTGAGCCAGC
CCCGGAGC

A nucleotide sequence encoding a micro-dystrophin protein optimized for cardiac muscle is further understood to include nucleotide sequences that are variants of any one of SEQ ID Nos. 7-12, 92-97, and 109-114. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID Nos. 7-12, 92-97, and 109-114.

In some embodiments, a micro-dystrophin protein optimized for cardiac muscle is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 92-97, and 109-114.

In some embodiments, a micro-dystrophin protein optimized for cardiac muscle is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 92-97, and 109-114.

In some embodiments, a micro-dystrophin protein optimized for cardiac muscle is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 92-97, and 109-114.

In some embodiments, it is desirable to avoid overexpression of micro-dystrophin proteins in the heart. Thus, in some embodiments, a nucleotide sequence encoding a micro-dystrophin protein optimized for cardiac muscle may be codon optimized for lower protein expression. In some embodiments, one or more codons (e.g., 1, 2, 3, 4, 5, 6, 7, 8, 9, 10, 11, 12, 13, 14, 15, 16, 17, 18, 19, 20, or more) are substituted for a rare codon.

### Skeletal Muscle Micro-Dystrophins

The present disclosure provides micro-dystrophin genes and proteins optimized for skeletal muscle. The inventors have found that certain combinations of domains from full-length dystrophin are beneficial for skeletal muscle.

In some embodiments, a micro-dystrophin protein optimized for skeletal muscle does not comprise spectrin-like repeat 2 and/or spectrin-like repeat 3 of dystrophin. In some embodiments, a micro-dystrophin protein does not comprise spectrin-like repeat 2. In some embodiments, a micro-dystrophin protein does not comprise spectrin-like repeat 3. In some embodiments, a micro-dystrophin protein does not comprise spectrin-like repeat 2 and spectrin-like repeat 3.

In some embodiments, a micro-dystrophin protein comprises optimized for skeletal muscle spectrin-like repeat 1 and spectrin-like repeat 2. In some embodiments, a micro-dystrophin protein optimized for skeletal muscle comprises spectrin-like repeat 1, spectrin-like repeat 2, and spectrin-like repeat 3.

In some embodiments, a micro-dystrophin protein optimized for skeletal muscle comprises an amino-terminal actin-binding domain, hinge domain 1, spectrin-like repeat 1, spectrin-like repeat 2, and spectrin-like repeat 17 of dystrophin. In some embodiments, the micro-dystrophin protein comprises spectrin-like repeat 16. In some embodiments, the micro-dystrophin protein does not comprise spectrin-like repeat 3 of dystrophin. In some embodiments, the micro-dystrophin protein comprises spectrin-like repeat 3 of dystrophin.

In some embodiments, a micro-dystrophin protein optimized for skeletal muscle comprises one or more of: hinge domain 3, spectrin-like repeat 22, spectrin-like repeat 23, spectrin-like repeat 24, hinge domain 4, and the dystroglycan binding site of dystrophin. In some embodiments, a micro-dystrophin protein optimized for skeletal muscle comprises spectrin-like repeat 23 and spectrin-like repeat 24. In some embodiments, a micro-dystrophin protein optimized for skeletal muscle comprises spectrin-like repeat 23 and hinge domain 4. In some embodiments, a micro-dystrophin protein optimized for skeletal muscle comprises spectrin-like repeat 23 and the dystroglycan binding site. In some embodiments, a micro-dystrophin protein optimized for skeletal muscle comprises spectrin-like repeat 24 and hinge domain 4. In some embodiments, a micro-dystrophin protein optimized for skeletal muscle comprises spectrin-like repeat 24 and the dystroglycan binding site. In some embodiments, a micro-dystrophin protein optimized for skeletal muscle comprises hinge domain 4 and the dystroglycan binding site. In some embodiments, a micro-dystrophin protein optimized for skeletal muscle comprises spectrin-like repeat 23, spectrin-like repeat 24 and hinge domain 4. In some embodiments, a micro-dystrophin protein optimized for skeletal muscle comprises spectrin-like repeat 23, spectrin-like repeat 24 and the dystroglycan binding site. In some embodiments, a micro-dystrophin protein optimized for skeletal muscle comprises spectrin-like repeat 23, hinge domain 4, and the dystroglycan binding site. In some embodiments, a micro-dystrophin protein optimized for skeletal muscle comprises spectrin-like repeat 24, hinge domain 4, and the dystroglycan binding site. In some embodiments, a micro-dystrophin protein optimized for skeletal muscle comprises spectrin-like repeat 23, spectrin-like repeat 24, hinge domain 4, and the dystroglycan binding site. In some embodiments, a micro-dystrophin protein optimized for skeletal muscle comprises hinge domain 3, spectrin-like repeat 22, hinge domain 4, and the dystroglycan binding site.

In some embodiments, a micro-dystrophin protein optimized for skeletal muscle comprises one or more syntrophin binding domains of dystrophin.

In some embodiments, a micro-dystrophin protein optimized for skeletal muscle comprises one or more coiled coil domains of dystrophin.

In some embodiments, a micro-dystrophin protein optimized for skeletal muscle comprises spectrin-like repeat 2 directly coupled to spectrin like repeat 16.

In some embodiments, a micro-dystrophin protein optimized for skeletal muscle comprises spectrin-like repeat 2 directly coupled to spectrin like repeat 17.

In some embodiments, a micro-dystrophin protein optimized for skeletal muscle comprises spectrin-like repeat 2 directly coupled to spectrin like repeat 23.

In some embodiments, a micro-dystrophin protein optimized for skeletal muscle comprises spectrin-like repeat 1 directly coupled spectrin-like repeat 15.

In some embodiments, a micro-dystrophin protein optimized for skeletal muscle comprises spectrin-like repeat 17 directly coupled to hinge domain 3. In some embodiments, a micro-dystrophin protein optimized for skeletal muscle comprises spectrin-like repeat 17 directly coupled to spectrin-like repeat 24. In some embodiments, a micro-dystrophin protein optimized for skeletal muscle comprises spectrin-like repeat 17 directly coupled to hinge domain 4.

In some embodiments, a micro-dystrophin protein optimized for skeletal muscle comprises the region of dystrophin that is C-terminal to the cysteine-rich portion of the C-terminal region (FIG. 2). In some embodiments, a micro-dystrophin optimized for skeletal muscle comprises region of dystrophin that is C-terminal to the dystroglycan binding site of dystrophin.

In some embodiments, a micro-dystrophin protein optimized for skeletal muscle comprises the region of dystrophin from spectrin-like repeat 24 to the C-terminus. In some embodiments, a micro-dystrophin protein optimized for skeletal muscle comprises the region of dystrophin from spectrin-like repeat 24 to the end of the proline rich region following the first coiled coil domain.

In some embodiments, a micro-dystrophin protein optimized for skeletal muscle comprises the region of dystrophin from hinge domain 4 to the C-terminus. In some embodiments, a micro-dystrophin protein optimized for skeletal muscle comprises the region of dystrophin from hinge domain 4 to the end of the proline rich region following the first coiled coil domain. In some embodiments, a micro-dystrophin protein optimized for skeletal muscle comprises the region of dystrophin from hinge domain 4 to the end of the second syntrophin binding domain.

In some embodiments, a micro-dystrophin protein of the disclosure comprises an amino-terminal actin-binding domain and hinge domain 1 region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 40. In some embodiments, a micro-dystrophin protein of the disclosure comprises a spectrin-like repeat 1 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 41. In some embodiments, a micro-dystrophin protein of the disclosure comprises a spectrin-like repeat 2 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 42. In some embodiments, a micro-dystrophin protein of the disclosure comprises a spectrin-like repeat 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 43. In some embodiments, a micro-dystrophin protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44. In some embodiments, a micro-dystrophin protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44 without the linker sequence. In some embodiments, a micro-dystrophin protein of the disclosure comprises a spectrin-like repeat 17 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 45. In some embodiments, a micro-dystrophin protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 46. In some embodiments, a micro-dystrophin protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 81. In some embodiments, a micro-dystrophin protein of the disclosure comprises a hinge domain 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 119. In some embodiments, a micro-dystrophin protein of the disclosure comprises a spectrin-like repeat 21 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 120. In some embodiments, a micro-dystrophin protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 121. In some embodiments, a micro-dystrophin protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 121 without the linker sequence. In some embodiments, a micro-dystrophin protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 47. In some embodiments, a micro-dystrophin protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 82. In some embodiments, a micro-dystrophin protein of the disclosure comprises a spectrin-like repeat 24 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 48. In some embodiments, a micro-dystrophin protein of the disclosure comprises a hinge domain 4 and dystroglycan binding site (also referred to herein as “trCterm”) comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 49. In some embodiments, a micro-dystrophin protein of the disclosure comprises a syntrophin binding region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 50. In some embodiments, a micro-dystrophin protein of the disclosure comprises a 1^{st} coiled coil region and proline rich region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51. In some embodiments, a micro-dystrophin protein of the disclosure comprises a 1^{st} coiled coil region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51 not including the proline rich region. In some embodiments, a micro-dystrophin protein of the disclosure comprises a region comprising the 2^{nd} coiled coil region to end of dystrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 52. In some embodiments, a micro-dystrophin protein of the disclosure comprises a an amino acid sequence that has up to 10 (e.g., 1, 2, 3, 4, 5, 6, 7, 8, 9, or 10) amino acid additions or deletions relative to a sequence provided herein (e.g., any one of SEQ ID NOs. 40-52, 81, 82, and 119-121).

Non-limiting examples of human micro-dystrophin proteins optimized for skeletal muscle are described below.

Nterm-R2_R16-R17_R24 to trCterm:
(SEQ ID NO: 13)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGREDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKWQRKIDETLERLQELQEATDELDLKLRQAEVIKGSWQPVGDLLIDSLQDHLEKVK
ALRGEIAPLKENVSHVNDLARQLTTLGIQLSPYNLSTLEDLNTRWKLLQVAVEDRVRQLHEAHRDFGPASQHFLSTS
VQGPWERAISPNKVPYYINHETQTTCWDHPKMTELYQSLADLNNVRFSAYRTAMKLRRLQKALCLDLLSLSAACDAL
DQHNLKQNDQPMDILQIINCLTTIYDRLEQEHNNLVNVPLCVDMCLNWLLNVYDTGRIGRIRVLSFKTGIISLCKAH
LEDKYRYLFKQVASSTGFCDQRRLGLLLHDSIQIPRQLGEVASFGGSNIEPSVRSCFQFANNKPEIEAALFLDWMRL
EPQSMVWLPVLHRVAAAETAKHQAKCNICKECPIIGFRYRSLKHFNYDICQSCFFSGRVAKGHKMHYPMVEYCTPTT
SGEDVRDFAKVLKNKFRTKRYFAKHPRMGYLPVQTVLEGDNMETP
Nterm-R2_R16-R17_H4 to end of syntrophin region:
(SEQ ID NO: 14)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKGPASQHFLSTSVQGPWERAISPNKVPYYINHETQTTCWDHPKMTELYQSLADLNN
VRFSAYRTAMKLRRLQKALCLDLLSLSAACDALDQHNLKQNDQPMDILQIINCLTTIYDRLEQEHNNLVNVPLCVDM
CLNWLLNVYDTGRTGRIRVLSFKTGIISLCKAHLEDKYRYLFKQVASSTGFCDQRRLGLLLHDSIQIPRQLGEVASF
GGSNIEPSVRSCFQFANNKPEIEAALFLDWMRLEPQSMVWLPVLHRVAAAETAKHQAKCNICKECPIIGFRYRSLKH
FNYDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDSPL
SQPRS
Nterm-R2_R16-R17_H4 to 1st coiled coil and proline rich region that follows:
(SEQ ID NO: 15)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKGPASQHFLSTSVQGPWERAISPNKVPYYINHETQTTCWDHPKMTELYQSLADLNN
VRFSAYRTAMKLRRLQKALCLDLLSLSAACDALDQHNLKQNDQPMDILQIINCLTTIYDRLEQEHNNLVNVPLCVDM
CLNWLLNVYDTGRIGRIRVLSFKTGIISLCKAHLEDKYRYLFKQVASSTGFCDQRRLGLLLHDSIQIPRQLGEVASF
GGSNIEPSVRSCFQFANNKPEIEAALFLDWMRLEPQSMVWLPVLHRVAAAETAKHQAKCNICKECPIIGFRYRSLKH
FNYDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDSPL
SQPRSPAQILISLESEERGELERILADLEEENRNLQAEYDRLKQQHEHKGLSPLPSPPEMMPTSPQSP
Nterm-R2_R16-R17_R24 to 1st coiled coil and proline rich region that follows:
(SEQ ID NO: 16)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKWQRKIDETLERLQELQEATDELDLKLRQAEVIKGSWQPVGDLLIDSLQDHLEKVK
ALRGEIAPLKENVSHVNDLARQLTTLGIQLSPYNLSTLEDLNTRWKLLQVAVEDRVRQLHEAHRDFGPASQHFLSTS
VQGPWERAISPNKVPYYINHETQTTCWDHPKMTELYQSLADLNNVRFSAYRTAMKLRRLQKALCLDLLSLSAACDAL
DQHNLKQNDQPMDILQIINCLTTIYDRLEQEHNNLVNVPLCVDMCLNWLLNVYDTGRTGRIRVLSFKTGIISLCKAH
LEDKYRYLFKQVASSTGFCDQRRLGLLLHDSIQIPRQLGEVASFGGSNIEPSVRSCFQFANNKPEIEAALFLDWMRL
EPQSMVWLPVLHRVAAAETAKHQAKCNICKECPIIGFRYRSLKHFNYDICQSCFFSGRVAKGHKMHYPMVEYCTPTT
SGEDVRDFAKVLKNKFRTKRYFAKHPRMGYLPVQTVLEGDNMETPVTLINFWPVDSAPASSPQLSHDDTHSRIEHYA
SRLAEMENSNGSYLNDSISPNESIDDEHLLIQHYCQSLNQDSPLSQPRSPAQILISLESEERGELERILADLEEENR
NLQAEYDRLKQQHEHKGLSPLPSPPEMMPTSPQSP
Nterm-R2_R23_R24 to end of dystrophin:
(SEQ ID NO: 17)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHLSLQELLVWLQLKDDELSRQAPIGGDEPAVQKQNDVHRAFKRELKTKEPVIMSTLETVR
IFLTEQPLEGLEKLYQEPRELPPEERAQNVTRLLRKQAEEVNTEWEKLNLHSADWQRKIDETLERLQELQEATDELD
LKLRQAEVIKGSWQPVGDLLIDSLQDHLEKVKALRGEIAPLKENVSHVNDLARQLTTLGIQLSPYNLSTLEDLNTRW
KLLQVAVEDRVRQLHEAHRDFGPASQHFLSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEHNNLVNVPLCVDMC
LNWLLNVYDTGRTGRIRVLSFKTGIISLCKAHLEDKYRYLFKQVASSTGFCDQRRLGLLLHDSIQIPRQLGEVASFG
GSNIEPSVRSCFQFANNKPEIEAALFLDWMRLEPQSMVWLPVLHRVAAAETAKHQAKCNICKECPIIGFRYRSLKHF
NYDICQSCFFSGRVAKGHKMHYPMVEYCTPTTSGEDVRDFAKVLKNKFRTKRYFAKHPRMGYLPVQTVLEGDNMETP
VTLINFWPVDSAPASSPQLSHDDTHSRIEHYASRLAEMENSNGSYLNDSISPNESIDDEHLLIQHYCQSLNQDSPLS
QPRSPAQILISLESEERGELERILADLEEENRNLQAEYDRLKQQHEHKGLSPLPSPPEMMPTSPQSPRDAELIAEAK
LLRQHKGRLEARMQILEDHNKQLESQLHRLRQLLEQPQAEAKVNGTTVSSPSTSLQRSDSSQPMLLRVVGSQTSDSM
GEEDLLSPPQDTSTGLEEVMEQLNNSFPSSRGRNTPGKPMREDTM
Nterm-R2_R23_R24 to end of dystrophin:
(SEQ ID NO: 98)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDASSDQWKRLHLSLQELLVWLQLKDDELSRQAPIGGDFPAVQKQNDVHRAFKRELKTKEPV
IMSTLETVRIFLTEQPLEGLEKLYQEPRELPPEERAQNVTRLLRKQAEEVNTEWEKLNLHSADWQRKIDETLERLQE
LQEATDELDLKLRQAEVIKGSWQPVGDLLIDSLQDHLEKVKALRGEIAPLKENVSHVNDLARQLTTLGIQLSPYNLS
TLEDLNTRWKLLQVAVEDRVRQLHEAHRDFGPASQHFLSTSVQGPWERAISPNKVPYYINHETQTTCWDHPKMTELY
QSLADLNNVRFSAYRTAMKLRRLQKALCLDLLSLSAACDALDQHNLKQNDQPMDILQIINCLTTIYDRLEQEHNNLV
NVPLCVDMCLNWLLNVYDTGRIGRIRVLSFKTGIISLCKAHLEDKYRYLFKQVASSTGFCDQRRLGLLLHDSIQIPR
QLGEVASFGGSNIEPSVRSCFQFANNKPEIEAALFLDWMRLEPQSMVWLPVLHRVAAAETAKHQAKCNICKECPIIG
FRYRSLKHFNYDICQSCFFSGRVAKGHKMHYPMVEYCTPTTSGEDVRDFAKVLKNKFRTKRYFAKHPRMGYLPVQTV
LEGDNMETPVTLINFWPVDSAPASSPQLSHDDTHSRIEHYASRLAEMENSNGSYLNDSISPNESIDDEHLLIQHYCQ
SLNQDSPLSQPRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSLQRSDSSQPMLLRV
VGSQTSDSMGEEDLLSPPQDTSTGLEEVMEQLNNSFPSSRGRNTPGKPMREDTM
Nterm-R2_R16-R17_H4 to end of dystrophin:
(SEQ ID NO: 18)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRILNATGEEIIQQSSKTDASILQEKLGSL
NLRWQEVCKQLSDRKKRLEEQKGPASQHFLSTSVQGPWERAISPNKVPYYINHETQTTCWDHPKMTELYQSLADLNN
VRFSAYRTAMKLRRLQKALCLDLLSLSAACDALDQHNLKQNDQPMDILQIINCLTTIYDRLEQEHNNLVNVPLCVDM
CLNWLLNVYDTGRIGRIRVLSFKTGIISLCKAHLEDKYRYLFKQVASSTGFCDQRRLGLLLHDSIQIPRQLGEVASF
GGSNIEPSVRSCFQFANNKPEIEAALFLDWMRLEPQSMVWLPVLHRVAAAETAKHQAKCNICKECPIIGFRYRSLKH
FNYDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDSPL
SQPRSPAQILISLESEERGELERILADLEEENRNLQAEYDRLKQQHEHKGLSPLPSPPEMMPTSPQSPRDAELIAEA
KLLRQHKGRLEARMQILEDHNKQLESQLHRLRQLLEQPQAEAKVNGTTVSSPSTSLQRSDSSQPMLLRVVGSQTSDS
MGEEDLLSPPQDTSTGLEEVMEQLNNSFPSSRGRNTPGKPMREDTM
Nterm-R1_R16-R17_R24 to end of dystrophin:
(SEQ ID NO: 19)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDHTVREETMMVM
TEDMPLEISYVPSTYLTEITHVSQALLEVEQLLNAPDLCAKDFEDLFKQEESLKNIKDSLQQSSGRIDIIHSKKTAA
LQSATPVERVKLQEALSQLDFQWEKVNKMYKDRQGRFDRSVEKWRRFHYDIKIFNQWLTEAEQFLRKTQIPENWEHA
KYKWYLKELQDGIGQRQTVVRTLNATGEEIIQQSSKTDASILQEKLGSLNLRWQEVCKQLSDRKKRLEEQKWQRKID
ETLERLQELQEATDELDLKLRQAEVIKGSWQPVGDLLIDSLQDHLEKVKALRGEIAPLKENVSHVNDLARQLTTLGI
QLSPYNLSTLEDLNTRWKLLQVAVEDRVRQLHEAHRDFGPASQHFLSTSVQGPWERAISPNKVPYYINHETQTTCWD
HPKMTELYQSLADLNNVRFSAYRTAMKLRRLQKALCLDLLSLSAACDALDQHNLKQNDQPMDILQIINCLTTIYDRL
EQEHNNLVNVPLCVDMCLNWLLNVYDTGRTGRIRVLSFKTGIISLCKAHLEDKYRYLFKQVASSTGFCDQRRLGLLL
HDSIQIPRQLGEVASFGGSNIEPSVRSCFQFANNKPEIEAALFLDWMRLEPQSMVWLPVLHRVAAAETAKHQAKCNI
CKECPIIGFRYRSLKHFNYDICQSCFFSGRVAKGHKMHYPMVEYCTPTTSGEDVRDFAKVLKNKFRTKRYFAKHPRM
GYLPVQTVLEGDNMETPVTLINFWPVDSAPASSPQLSHDDTHSRIEHYASRLAEMENSNGSYLNDSISPNESIDDEH
LLIQHYCQSLNQDSPLSQPRSPAQILISLESEERGELERILADLEEENRNLQAEYDRLKQQHEHKGLSPLPSPPEMM
PTSPQSPRDAELIAEAKLLRQHKGRLEARMQILEDHNKQLESQLHRLRQLLEQPQAEAKVNGTTVSSPSTSLQRSDS
SQPMLLRVVGSQTSDSMGEEDLLSPPQDTSTGLEEVMEQLNNSFPSSRGRNTPGKPMREDTM
Nterm-R2_R16-R17_R24 to end of dystrophin:
(SEQ ID NO: 20)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKWQRKIDETLERLQELQEATDELDLKLRQAEVIKGSWQPVGDLLIDSLQDHLEKVK
ALRGEIAPLKENVSHVNDLARQLTTLGIQLSPYNLSTLEDLNTRWKLLQVAVEDRVRQLHEAHRDFGPASQHFLSTS
VQGPWERAISPNKVPYYINHETQTTCWDHPKMTELYQSLADLNNVRFSAYRTAMKLRRLQKALCLDLLSLSAACDAL
DQHNLKQNDQPMDILQIINCLTTIYDRLEQEHNNLVNVPLCVDMCLNWLLNVYDTGRTGRIRVLSFKTGIISLCKAH
LEDKYRYLFKQVASSTGFCDQRRLGLLLHDSIQIPRQLGEVASFGGSNIEPSVRSCFQFANNKPEIEAALFLDWMRL
EPQSMVWLPVLHRVAAAETAKHQAKCNICKECPIIGFRYRSLKHFNYDICQSCFFSGRVAKGHKMHYPMVEYCTPTT
SGEDVRDFAKVLKNKFRTKRYFAKHPRMGYLPVQTVLEGDNMETPVTLINFWPVDSAPASSPQLSHDDTHSRIEHYA
SRLAEMENSNGSYLNDSISPNESIDDEHLLIQHYCQSLNQDSPLSQPRSPAQILISLESEERGELERILADLEEENR
NLQAEYDRLKQQHEHKGLSPLPSPPEMMPTSPQSPRDAELIAEAKLLRQHKGRLEARMQILEDHNKQLESQLHRLRQ
LLEQPQAEAKVNGTTVSSPSTSLQRSDSSQPMLLRVVGSQTSDSMGEEDLLSPPQDTSTGLEEVMEQLNNSFPSSRG
RNTPGKPMREDTM
Nterm-R2_R16-R17_R24 to end of syntrophin region:
(SEQ ID NO: 21)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDHTVREETMMVMTEDMPLEISYVPSTYLTEITHVSQALLEVEQLLNAPDLCAKDFEDLFKQ
EESLKNIKDSLQQSSGRIDIIHSKKTAALQSATPVERVKLQEALSQLDFQWEKVNKMYKDRQGRFDRSVEKWRRFHY
DIKIFNQWLTEAEQFLRKTQIPENWEHAKYKWYLKELQDGIGQRQTVVRTLNATGEEIIQQSSKTDASILQEKLGSL
NLRWQEVCKQLSDRKKRLEEQKWQRKIDETLERLQELQEATDELDLKLRQAEVIKGSWQPVGDLLIDSLQDHLEKVK
ALRGEIAPLKENVSHVNDLARQLTTLGIQLSPYNLSTLEDLNTRWKLLQVAVEDRVRQLHEAHRDFGPASQHFLSTS
VQGPWERAISPNKVPYYINHETQTTCWDHPKMTELYQSLADLNNVRFSAYRTAMKLRRLQKALCLDLLSLSAACDAL
DQHNLKQNDQPMDILQIINCLTTIYDRLEQEHNNLVNVPLCVDMCLNWLLNVYDTGRTGRIRVLSFKTGIISLCKAH
LEDKYRYLFKQVASSTGFCDQRRLGLLLHDSIQIPRQLGEVASFGGSNIEPSVRSCFQFANNKPEIEAALFLDWMRL
EPQSMVWLPVLHRVAAAETAKHQAKCNICKECPIIGFRYRSLKHFNYDICQSCFFSGRVAKGHKMHYPMVEYCTPTT
SGEDVRDFAKVLKNKFRTKRYFAKHPRMGYLPVQTVLEGDNMETPVTLINFWPVDSAPASSPQLSHDDTHSRIEHYA
SRLAEMENSNGSYLNDSISPNESIDDEHLLIQHYCQSLNQDSPLSQPRS
Nterm-R2_R17_H3_R22_H4 to end of syntrophin region:
(SEQ ID NO: 115)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAPEDKSFGSSLMESEVNLDRYQTALEEVLSWLLSAEDTLQAQGEISNDVEVVKDQFHTHEGY
MMDLTAHQGRVGNILQLGSKLIGTGKLSEDEETEVQEQMNLLNSRWECLRVASMEKQSNLHRVLMDLQNQKLKELND
WLTKTEERTRKMEEEPLGPDLEDLKRQVQQHKVLQEDLEQEQVRVNSLTHMVVVVDESSGDHATAALEEQLKVLGDR
WANICRWTEDRWVLLQDRFDRSVEKWRRFHYDIKIFNQWLTEAEQFLRKTQIPENWEHAKYKWYLKELQDGIGQRQT
VVRTLNATGEEIIQQSSKTDASILQEKLGSLNLRWQEVCKQLSDRKKRLEEQKQPDLAPGLTTIGASPTQTVTLVTQ
PVVTKETAISKLEMPSSLMLEVPEETHRLLQQFPLDLEKFLAWLTEAETTANVLQDATRKERLLEDSKGVKELMKQW
QDLQGEIEAHTDVYHNLDENSQKILRSLEGSDDAVLLQRRLDNMNFKWSELRKKSLNIRSHLEASSDQWKRLGPASQ
HFLSTSVQGPWERAISPNKVPYYINHETQTTCWDHPKMTELYQSLADLNNVRFSAYRTAMKLRRLQKALCLDLLSLS
AACDALDQHNLKQNDQPMDILQIINCLTTIYDRLEQEHNNLVNVPLCVDMCLNWLLNVYDTGRTGRIRVLSFKTGII
SLCKAHLEDKYRYLFKQVASSTGFCDQRRLGLLLHDSIQIPRQLGEVASFGGSNIEPSVRSCFQFANNKPEIEAALF
LDWMRLEPQSMVWLPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSAPASSPQLSHDDTHS
RIEHYASRLAEMENSNGSYLNDSISPNESIDDEHLLIQHYCQSLNQDSPLSQPRS

A micro-dystrophin protein optimized for skeletal muscle is further understood to include proteins that are variants of any one of SEQ ID Nos. 13-21, 98, and 115. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID Nos. 13-21, 98, and 115.

In some embodiments, a micro-dystrophin protein optimized for skeletal muscle comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 98, and 115.

In some embodiments, a micro-dystrophin protein optimized for skeletal muscle consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 98, and 115.

In some embodiments, a micro-dystrophin protein optimized for skeletal muscle consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 98, and 115.

In some embodiments, functional variants or modified forms of micro-dystrophin proteins optimized for skeletal muscle include fusion proteins having at least a portion of the micro-dystrophin protein and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

The disclosure also provides nucleic acids encoding any of the micro-dystrophins optimized for skeletal muscle described herein. Such nucleic acids may be DNA or RNA molecules. These nucleic acids may be used, for example, in methods for making micro-dystrophins or as direct therapeutic agents in a gene therapy approach.

Non-limiting examples of nucleotide sequences encoding human micro-dystrophin proteins optimized for skeletal muscle are described below.

Nterm-R2_R16-R17_R24 to trCterm:
(SEQ ID NO: 22)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGTGGCAGAGAAA
AATAGATGAGACCCTTGAAAGACTCCGGGAACTTCAAGAGGCCACGGATGAGCTGGACCTCAAGCTGCGCCAAGCTG
AGGTGATCAAGGGATCCTGGCAGCCCGTGGGCGATCTCCTCATTGACTCTCTCCAAGATCACCTCGAGAAAGTCAAG
GCACTTCGAGGAGAAATTGCGCCTCTGAAAGAGAACGTGAGCCACGTCAATGACCTTGCTCGCCAGCTTACCACTTT
GGGCATTCAGCTCTCACCGTATAACCTCAGCACTCTGGAAGACCTGAACACCAGATGGAAGCTTCTGCAGGTGGCCG
TCGAGGACCGAGTCAGGCAGCTGCATGAAGCCCACAGGGACTTTGGTCCAGCATCTCAGCACTTTCTTTCCACGTCT
GTCCAGGGTCCCTGGGAGAGAGCCATCTCGCCAAACAAAGTGCCCTACTATATCAACCACGAGACTCAAACAACTTG
CTGGGACCATCCCAAAATGACAGAGCTCTACCAGTCTTTAGCTGACCTGAATAATGTCAGATTCTCAGCTTATAGGA
CTGCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTATGA
CCGCCTGGAGCAAGAGCACAACAATTTGGTCAACGTCCCTCTCTGCGTGGATATGTGTCTGAACTGGCTGCTGAATG
TTTATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGCCT
CCTTCTGCATGATTCTATCCAAATTCCAAGACAGTTGGGTGAAGTTGCATCCTTTGGGGGCAGTAACATTGAGCCAA
GTGTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAATG
TAACATCTGCAAAGAGTGTCCAATCATTGGATTCAGGTACAGGAGTCTAAAGCACTTTAATTATGACATCTGCCAAA
GCTGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCATCC
CCGAATGGGCTACCTGCCAGTGCAGACTGTCTTAGAGGGGGACAACATGGAAACTCCC
Nterm-R2_R16-R17_H4 to end of syntrophin region:
(SEQ ID NO: 23)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGGGTCCAGCATC
TCAGCACTTTCTTTCCACGTCTGTCCAGGGTCCCTGGGAGAGAGCCATCTCGCCAAACAAAGTGCCCTACTATATCA
ACCACGAGACTCAAACAACTTGCTGGGACCATCCCAAAATGACAGAGCTCTACCAGTCTTTAGCTGACCTGAATAAT
GTCAGATTCTCAGCTTATAGGACTGCCATGAAACTCCGAAGACTGCAGAAGGCCCTTTGCTTGGATCTCTTGAGCCT
GTCAGCTGCATGTGATGCCTTGGACCAGCACAACCTCAAGCAAAATGACCAGCCCATGGATATCCTGCAGATTATTA
ATTGTTTGACCACTATTTATGACCGCCTGGAGCAAGAGCACAACAATTTGGTCAACGTCCCTCTCTGCGTGGATATG
TGTCTGAACTGGCTGCTGAATGTTTATGATACGGGACGAACAGGGAGGATCCGTGTCCTGTCTTTTAAAACTGGCAT
CATTTCCCTGTGTAAAGCACATTTGGAAGACAAGTACAGATACCTTTTCAAGCAAGTGGCAAGTTCAACAGGATTTT
GTGACCAGCGCAGGCTGGGCCTCCTTCTGCATGATTCTATCCAAATTCCAAGACAGTTGGGTGAAGTTGCATCCTTT
GGGGGCAGTAACATTGAGCCAAGTGTCCGGAGCTGCTTCCAATTTGCTAATAATAAGCCAGAGATCGAAGCGGCCCT
CTTCCTAGACTGGATGAGACTGGAACCCCAGTCCATGGTGTGGCTGCCCGTCCTGCACAGAGTGGCTGCTGCAGAAA
CTGCCAAGCATCAGGCCAAATGTAACATCTGCAAAGAGTGTCCAATCATTGGATTCAGGTACAGGAGTCTAAAGCAC
TTTAATTATGACATCTGCCAAAGCTGCTTTTTTTCTGGTCGAGTTGCAAAAGGCCATAAAATGCACTATCCCATGGT
GGAATATTGCACTCCGACTACATCAGGAGAAGATGTTCGAGACTTTGCCAAGGTACTAAAAAACAAATTTCGAACCA
AAAGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACTCA
TTCACGCATTGAACATTATGCTAGCAGGCTAGCAGAAATGGAAAACAGCAATGGATCTTATCTAAATGATAGCATCT
CTCCTAATGAGAGCATAGATGATGAACATTTGTTAATCCAGCATTACTGCCAAAGTTTGAACCAGGACTCCCCCCTG
AGCCAGCCTCGTAGT
Nterm-R2_R16-R17_H4 to 1st coiled coil and proline rich region that follows:
(SEQ ID NO: 24)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGGGTCCAGCATC
TCAGCACTTTCTTTCCACGTCTGTCCAGGGTCCCTGGGAGAGAGCCATCTCGCCAAACAAAGTGCCCTACTATATCA
ACCACGAGACTCAAACAACTTGCTGGGACCATCCCAAAATGACAGAGCTCTACCAGTCTTTAGCTGACCTGAATAAT
GTCAGATTCTCAGCTTATAGGACTGCCATGAAACTCCGAAGACTGCAGAAGGCCCTTTGCTTGGATCTCTTGAGCCT
GTCAGCTGCATGTGATGCCTTGGACCAGCACAACCTCAAGCAAAATGACCAGCCCATGGATATCCTGCAGATTATTA
ATTGTTTGACCACTATTTATGACCGCCTGGAGCAAGAGCACAACAATTTGGTCAACGTCCCTCTCTGCGTGGATATG
TGTCTGAACTGGCTGCTGAATGTTTATGATACGGGACGAACAGGGAGGATCCGTGTCCTGTCTTTTAAAACTGGCAT
CATTTCCCTGTGTAAAGCACATTTGGAAGACAAGTACAGATACCTTTTCAAGCAAGTGGCAAGTTCAACAGGATTTT
GTGACCAGCGCAGGCTGGGCCTCCTTCTGCATGATTCTATCCAAATTCCAAGACAGTTGGGTGAAGTTGCATCCTTT
GGGGGCAGTAACATTGAGCCAAGTGTCCGGAGCTGCTTCCAATTTGCTAATAATAAGCCAGAGATCGAAGCGGCCCT
CTTCCTAGACTGGATGAGACTGGAACCCCAGTCCATGGTGTGGCTGCCCGTCCTGCACAGAGTGGCTGCTGCAGAAA
CTGCCAAGCATCAGGCCAAATGTAACATCTGCAAAGAGTGTCCAATCATTGGATTCAGGTACAGGAGTCTAAAGCAC
TTTAATTATGACATCTGCCAAAGCTGCTTTTTTTCTGGTCGAGTTGCAAAAGGCCATAAAATGCACTATCCCATGGT
GGAATATTGCACTCCGACTACATCAGGAGAAGATGTTCGAGACTTTGCCAAGGTACTAAAAAACAAATTTCGAACCA
AAAGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACTCA
TTCACGCATTGAACATTATGCTAGCAGGCTAGCAGAAATGGAAAACAGCAATGGATCTTATCTAAATGATAGCATCT
CTCCTAATGAGAGCATAGATGATGAACATTTGTTAATCCAGCATTACTGCCAAAGTTTGAACCAGGACTCCCCCCTG
AGCCAGCCTCGTAGTCCTGCCCAGATCTTGATTTCCTTAGAGAGTGAGGAAAGAGGGGAGCTAGAGAGAATCCTAGC
AGATCTTGAGGAAGAAAACAGGAATCTGCAAGCAGAATATGACCGTCTAAAGCAGCAGCACGAACATAAAGGCCTGT
CCCCACTGCCGTCCCCTCCTGAAATGATGCCCACCTCTCCCCAGAGTCCC
Nterm-R2_R16-R17_R24 to 1st coiled coil and proline rich region that follows:
(SEQ ID NO: 25)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGTGGCAGAGAAA
AATAGATGAGACCCTTGAAAGACTCCGGGAACTTCAAGAGGCCACGGATGAGCTGGACCTCAAGCTGCGCCAAGCTG
AGGTGATCAAGGGATCCTGGCAGCCCGTGGGCGATCTCCTCATTGACTCTCTCCAAGATCACCTCGAGAAAGTCAAG
GCACTTCGAGGAGAAATTGCGCCTCTGAAAGAGAACGTGAGCCACGTCAATGACCTTGCTCGCCAGCTTACCACTTT
GGGCATTCAGCTCTCACCGTATAACCTCAGCACTCTGGAAGACCTGAACACCAGATGGAAGCTTCTGCAGGTGGCCG
TCGAGGACCGAGTCAGGCAGCTGCATGAAGCCCACAGGGACTTTGGTCCAGCATCTCAGCACTTTCTTTCCACGTCT
GTCCAGGGTCCCTGGGAGAGAGCCATCTCGCCAAACAAAGTGCCCTACTATATCAACCACGAGACTCAAACAACTTG
CTGGGACCATCCCAAAATGACAGAGCTCTACCAGTCTTTAGCTGACCTGAATAATGTCAGATTCTCAGCTTATAGGA
CTGCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTATGA
CCGCCTGGAGCAAGAGCACAACAATTTGGTCAACGTCCCTCTCTGCGTGGATATGTGTCTGAACTGGCTGCTGAATG
TTTATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGCCT
CCTTCTGCATGATTCTATCCAAATTCCAAGACAGTTGGGTGAAGTTGCATCCTTTGGGGGCAGTAACATTGAGCCAA
GTGTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAATG
TAACATCTGCAAAGAGTGTCCAATCATTGGATTCAGGTACAGGAGTCTAAAGCACTTTAATTATGACATCTGCCAAA
GCTGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCATCC
CCGAATGGGCTACCTGCCAGTGCAGACTGTCTTAGAGGGGGACAACATGGAAACTCCCGTTACTCTGATCAACTTCT
GGCCAGTAGATTCTGCGCCTGCCTCGTCCCCTCAGCTTTCACACGATGATACTCATTCACGCATTGAACATTATGCT
AGCAGGCTAGCAGAAATGGAAAACAGCAATGGATCTTATCTAAATGATAGCATCTCTCCTAATGAGAGCATAGATGA
TGAACATTTGTTAATCCAGCATTACTGCCAAAGTTTGAACCAGGACTCCCCCCTGAGCCAGCCTCGTAGTCCTGCCC
AGATCTTGATTTCCTTAGAGAGTGAGGAAAGAGGGGAGCTAGAGAGAATCCTAGCAGATCTTGAGGAAGAAAACAGG
AATCTGCAAGCAGAATATGACCGTCTAAAGCAGCAGCACGAACATAAAGGCCTGTCCCCACTGCCGTCCCCTCCTGA
AATGATGCCCACCTCTCCCCAGAGTCCC
Nterm-R2_R23_R24 to end of dystrophin:
(SEQ ID NO: 26)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACCTTTCTCTGCAGGAACTTCTGGT
GTGGCTACAGCTGAAAGATGATGAATTAAGCCGGCAGGCACCTATTGGAGGCGACTTTCCAGCAGTTCAGAAGCAGA
ACGATGTACATAGGGCCTTCAAGAGGGAATTGAAAACTAAAGAACCTGTAATCATGAGTACTCTTGAGACTGTACGA
ATATTTCTGACAGAGCAGCCTTTGGAAGGACTAGAGAAACTCTACCAGGAGCCCAGAGAGCTGCCTCCTGAGGAGAG
AGCCCAGAATGTCACTCGGCTTCTACGAAAGCAGGCTGAGGAGGTCAATACTGAGTGGGAAAAATTGAACCTGCACT
CCGCTGACTGGCAGAGAAAAATAGATGAGACCCTTGAAAGACTCCGGGAACTTCAAGAGGCCACGGATGAGCTGGAC
CTCAAGCTGCGCCAAGCTGAGGTGATCAAGGGATCCTGGCAGCCCGTGGGCGATCTCCTCATTGACTCTCTCCAAGA
TCACCTCGAGAAAGTCAAGGCACTTCGAGGAGAAATTGCGCCTCTGAAAGAGAACGTGAGCCACGTCAATGACCTTG
CTCGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTTGGTCCAGCATCTCA
GCACTTTCTTTCCACGTCTGTCCAGGGTCCCTGGGAGAGAGCCATCTCGCCAAACAAAGTGCCCTACTATATCAACC
ACGAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTGTC
AGCTGCATGTGATGCCTTGGACCAGCACAACCTCAAGCAAAATGACCAGCCCATGGATATCCTGCAGATTATTAATT
GTTTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATCAT
TTCCCTGTGTAAAGCACATTTGGAAGACAAGTACAGATACCTTTTCAAGCAAGTGGCAAGTTCAACAGGATTTTGTG
ACCAGCGCAGGCTGGGCCTCCTTCTGCATGATTCTATCCAAATTCCAAGACAGTTGGGTGAAGTTGCATCCTTTGGG
GGCAGTAACATTGAGCCAAGTGTCCGGAGCTGCTTCCAATTTGCTAATAATAAGCCAGAGATCGAAGCGGCCCTCTT
CCTAGACTGGATGAGACTGGAACCCCAGTCCATGGTGTGGCTGCCCGTCCTGCACAGAGTGGCTGCTGCAGAAACTG
CCAAGCATCAGGCCAAATGTAACATCTGCAAAGAGTGTCCAATCATTGGATTCAGGTACAGGAGTCTAAAGCACTTT
AATTATGACATCTGCCAAAGCTGCTTTTTTTCTGGTCGAGTTGCAAAAGGCCATAAAATGCACTATCCCATGGTGGA
ATATTGCACTCCGACTACATCAGGAGAAGATGTTCGAGACTTTGCCAAGGTACTAAAAAACAAATTTCGAACCAAAA
GGTATTTTGCGAAGCATCCCCGAATGGGCTACCTGCCAGTGCAGACTGTCTTAGAGGGGGACAACATGGAAACTCCC
GTTACTCTGATCAACTTCTGGCCAGTAGATTCTGCGCCTGCCTCGTCCCCTCAGCTTTCACACGATGATACTCATTC
ACGCATTGAACATTATGCTAGCAGGCTAGCAGAAATGGAAAACAGCAATGGATCTTATCTAAATGATAGCATCTCTC
CTAATGAGAGCATAGATGATGAACATTTGTTAATCCAGCATTACTGCCAAAGTTTGAACCAGGACTCCCCCCTGAGC
CAGCCTCGTAGTCCTGCCCAGATCTTGATTTCCTTAGAGAGTGAGGAAAGAGGGGAGCTAGAGAGAATCCTAGCAGA
TCTTGAGGAAGAAAACAGGAATCTGCAAGCAGAATATGACCGTCTAAAGCAGCAGCACGAACATAAAGGCCTGTCCC
CACTGCCGTCCCCTCCTGAAATGATGCCCACCTCTCCCCAGAGTCCCCGGGATGCTGAGCTCATTGCTGAGGCCAAG
CTACTGCGTCAACACAAAGGCCGCCTGGAAGCCAGGATGCAAATCCTGGAAGACCACAATAAACAGCTGGAGTCACA
GTTACACAGGCTAAGGCAGCTGCTGGAGCAACCCCAGGCAGAGGCCAAAGTGAATGGCACAACGGTGTCCTCTCCTT
CTACCTCTCTACAGAGGTCCGACAGCAGTCAGCCTATGCTGCTCCGAGTGGTTGGCAGTCAAACTTCGGACTCCATG
GGTGAGGAAGATCTTCTCAGTCCTCCCCAGGACACAAGCACAGGGTTAGAGGAGGTGATGGAGCAACTCAACAACTC
CTTCCCTAGTTCAAGAGGAAGAAATACCCCTGGAAAGCCAATGAGAGAGGACACAATG
Nterm-R2_R23_R24 to end of dystrophin:
(SEQ ID NO: 99)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACGCCAGTTCTGACCAGTGGAAGCGTCT
GCACCTTTCTCTGCAGGAACTTCTGGTGTGGCTACAGCTGAAAGATGATGAATTAAGCCGGCAGGCACCTATTGGAG
GCGACTTTCCAGCAGTTCAGAAGCAGAACGATGTACATAGGGCCTTCAAGAGGGAATTGAAAACTAAAGAACCTGTA
ATCATGAGTACTCTTGAGACTGTACGAATATTTCTGACAGAGCAGCCTTTGGAAGGACTAGAGAAACTCTACCAGGA
GCCCAGAGAGCTGCCTCCTGAGGAGAGAGCCCAGAATGTCACTCGGCTTCTACGAAAGCAGGCTGAGGAGGTCAATA
CTGAGTGGGAAAAATTGAACCTGCACTCCGCTGACTGGCAGAGAAAAATAGATGAGACCCTTGAAAGACTCCGGGAA
CTTCAAGAGGCCACGGATGAGCTGGACCTCAAGCTGCGCCAAGCTGAGGTGATCAAGGGATCCTGGCAGCCCGTGGG
CGATCTCCTCATTGACTCTCTCCAAGATCACCTCGAGAAAGTCAAGGCACTTCGAGGAGAAATTGCGCCTCTGAAAG
AGAACGTGAGCCACGTCAATGACCTTGCTCGCCAGCTTACCACTTTGGGCATTCAGCTCTCACCGTATAACCTCAGC
ACTCTGGAAGACCTGAACACCAGATGGAAGCTTCTGCAGGTGGCCGTCGAGGACCGAGTCAGGCAGCTGCATGAAGC
CCACAGGGACTTTGGTCCAGCATCTCAGCACTTTCTTTCCACGTCTGTCCAGGGTCCCTGGGAGAGAGCCATCTCGC
CAAACAAAGTGCCCTACTATATCAACCACGAGACTCAAACAACTTGCTGGGACCATCCCAAAATGACAGAGCTCTAC
CAGTCTTTAGCTGACCTGAATAATGTCAGATTCTCAGCTTATAGGACTGCCATGAAACTCCGAAGACTGCAGAAGGC
CCTTTGCTTGGATCTCTTGAGCCTGTCAGCTGCATGTGATGCCTTGGACCAGCACAACCTCAAGCAAAATGACCAGC
CCATGGATATCCTGCAGATTATTAATTGTTTGACCACTATTTATGACCGCCTGGAGCAAGAGCACAACAATTTGGTC
AACGTCCCTCTCTGCGTGGATATGTGTCTGAACTGGCTGCTGAATGTTTATGATACGGGACGAACAGGGAGGATCCG
TGTCCTGTCTTTTAAAACTGGCATCATTTCCCTGTGTAAAGCACATTTGGAAGACAAGTACAGATACCTTTTCAAGC
AAGTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAATAA
TAAGCCAGAGATCGAAGCGGCCCTCTTCCTAGACTGGATGAGACTGGAACCCCAGTCCATGGTGTGGCTGCCCGTCC
TGCACAGAGTGGCTGCTGCAGAAACTGCCAAGCATCAGGCCAAATGTAACATCTGCAAAGAGTGTCCAATCATTGGA
TTCAGGTACAGGAGTCTAAAGCACTTTAATTATGACATCTGCCAAAGCTGCTTTTTTTCTGGTCGAGTTGCAAAAGG
CCATAAAATGCACTATCCCATGGTGGAATATTGCACTCCGACTACATCAGGAGAAGATGTTCGAGACTTTGCCAAGG
TACTAAAAAACAAATTTCGAACCAAAAGGTATTTTGCGAAGCATCCCCGAATGGGCTACCTGCCAGTGCAGACTGTC
TTAGAGGGGGACAACATGGAAACTCCCGTTACTCTGATCAACTTCTGGCCAGTAGATTCTGCGCCTGCCTCGTCCCC
TCAGCTTTCACACGATGATACTCATTCACGCATTGAACATTATGCTAGCAGGCTAGCAGAAATGGAAAACAGCAATG
GATCTTATCTAAATGATAGCATCTCTCCTAATGAGAGCATAGATGATGAACATTTGTTAATCCAGCATTACTGCCAA
AGTTTGAACCAGGACTCCCCCCTGAGCCAGCCTCGTAGTCCTGCCCAGATCTTGATTTCCTTAGAGAGTGAGGAAAG
AGGGGAGCTAGAGAGAATCCTAGCAGATCTTGAGGAAGAAAACAGGAATCTGCAAGCAGAATATGACCGTCTAAAGC
AGCAGCACGAACATAAAGGCCTGTCCCCACTGCCGTCCCCTCCTGAAATGATGCCCACCTCTCCCCAGAGTCCCCGG
GATGCTGAGCTCATTGCTGAGGCCAAGCTACTGCGTCAACACAAAGGCCGCCTGGAAGCCAGGATGCAAATCCTGGA
AGACCACAATAAACAGCTGGAGTCACAGTTACACAGGCTAAGGCAGCTGCTGGAGCAACCCCAGGCAGAGGCCAAAG
TGAATGGCACAACGGTGTCCTCTCCTTCTACCTCTCTACAGAGGTCCGACAGCAGTCAGCCTATGCTGCTCCGAGTG
GTTGGCAGTCAAACTTCGGACTCCATGGGTGAGGAAGATCTTCTCAGTCCTCCCCAGGACACAAGCACAGGGTTAGA
GGAGGTGATGGAGCAACTCAACAACTCCTTCCCTAGTTCAAGAGGAAGAAATACCCCTGGAAAGCCAATGAGAGAGG
ACACAATG
Nterm-R2_R16-R17_H4 to end of dystrophin:
(SEQ ID NO: 27)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGGGTCCAGCATC
TCAGCACTTTCTTTCCACGTCTGTCCAGGGTCCCTGGGAGAGAGCCATCTCGCCAAACAAAGTGCCCTACTATATCA
ACCACGAGACTCAAACAACTTGCTGGGACCATCCCAAAATGACAGAGCTCTACCAGTCTTTAGCTGACCTGAATAAT
GTCAGATTCTCAGCTTATAGGACTGCCATGAAACTCCGAAGACTGCAGAAGGCCCTTTGCTTGGATCTCTTGAGCCT
GTCAGCTGCATGTGATGCCTTGGACCAGCACAACCTCAAGCAAAATGACCAGCCCATGGATATCCTGCAGATTATTA
ATTGTTTGACCACTATTTATGACCGCCTGGAGCAAGAGCACAACAATTTGGTCAACGTCCCTCTCTGCGTGGATATG
TGTCTGAACTGGCTGCTGAATGTTTATGATACGGGACGAACAGGGAGGATCCGTGTCCTGTCTTTTAAAACTGGCAT
CATTTCCCTGTGTAAAGCACATTTGGAAGACAAGTACAGATACCTTTTCAAGCAAGTGGCAAGTTCAACAGGATTTT
GTGACCAGCGCAGGCTGGGCCTCCTTCTGCATGATTCTATCCAAATTCCAAGACAGTTGGGTGAAGTTGCATCCTTT
GGGGGCAGTAACATTGAGCCAAGTGTCCGGAGCTGCTTCCAATTTGCTAATAATAAGCCAGAGATCGAAGCGGCCCT
CTTCCTAGACTGGATGAGACTGGAACCCCAGTCCATGGTGTGGCTGCCCGTCCTGCACAGAGTGGCTGCTGCAGAAA
CTGCCAAGCATCAGGCCAAATGTAACATCTGCAAAGAGTGTCCAATCATTGGATTCAGGTACAGGAGTCTAAAGCAC
TTTAATTATGACATCTGCCAAAGCTGCTTTTTTTCTGGTCGAGTTGCAAAAGGCCATAAAATGCACTATCCCATGGT
GGAATATTGCACTCCGACTACATCAGGAGAAGATGTTCGAGACTTTGCCAAGGTACTAAAAAACAAATTTCGAACCA
AAAGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACTCA
TTCACGCATTGAACATTATGCTAGCAGGCTAGCAGAAATGGAAAACAGCAATGGATCTTATCTAAATGATAGCATCT
CTCCTAATGAGAGCATAGATGATGAACATTTGTTAATCCAGCATTACTGCCAAAGTTTGAACCAGGACTCCCCCCTG
AGCCAGCCTCGTAGTCCTGCCCAGATCTTGATTTCCTTAGAGAGTGAGGAAAGAGGGGAGCTAGAGAGAATCCTAGC
AGATCTTGAGGAAGAAAACAGGAATCTGCAAGCAGAATATGACCGTCTAAAGCAGCAGCACGAACATAAAGGCCTGT
CCCCACTGCCGTCCCCTCCTGAAATGATGCCCACCTCTCCCCAGAGTCCCCGGGATGCTGAGCTCATTGCTGAGGCC
AAGCTACTGCGTCAACACAAAGGCCGCCTGGAAGCCAGGATGCAAATCCTGGAAGACCACAATAAACAGCTGGAGTC
ACAGTTACACAGGCTAAGGCAGCTGCTGGAGCAACCCCAGGCAGAGGCCAAAGTGAATGGCACAACGGTGTCCTCTC
CTTCTACCTCTCTACAGAGGTCCGACAGCAGTCAGCCTATGCTGCTCCGAGTGGTTGGCAGTCAAACTTCGGACTCC
ATGGGTGAGGAAGATCTTCTCAGTCCTCCCCAGGACACAAGCACAGGGTTAGAGGAGGTGATGGAGCAACTCAACAA
CTCCTTCCCTAGTTCAAGAGGAAGAAATACCCCTGGAAAGCCAATGAGAGAGGACACAATG
Nterm-R1_R16-R17_R24 to end of dystrophin:
(SEQ ID NO: 28)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCACACTGTCCGTGAAGAAACGATGATGGTGATG
ACTGAAGACATGCCTTTGGAAATTTCTTATGTGCCTTCTACTTATTTGACTGAAATCACTCATGTCTCACAAGCCCT
ATTAGAAGTGGAACAACTTCTCAATGCTCCTGACCTCTGTGCTAAGGACTTTGAAGATCTCTTTAAGCAAGAGGAGT
CTCTGAAGAATATAAAAGATAGTCTACAACAAAGCTCAGGTCGGATTGACATTATTCATAGCAAGAAGACAGCAGCA
TTGCAAAGTGCAACGCCTGTGGAAAGGGTGAAGCTACAGGAAGCTCTCTCCCAGCTTGATTTCCAATGGGAAAAAGT
TAACAAAATGTACAAGGACCGACAAGGGCGATTTGACAGATCTGTTGAGAAATGGCGGCGTTTTCATTATGATATAA
AGATATTTAATCAGTGGCTAACAGAAGCTGAACAGTTTCTCAGAAAGACACAAATTCCTGAGAATTGGGAACATGCT
AAATACAAATGGTATCTTAAGGAACTCCAGGATGGCATTGGGCAGCGGCAAACTGTTGTCAGAACATTGAATGCAAC
TGGGGAAGAAATAATTCAGCAATCCTCAAAAACAGATGCCAGTATTCTACAGGAAAAATTGGGAAGCCTGAATCTGC
GGTGGCAGGAGGTCTGCAAACAGCTGTCAGACAGAAAAAAGAGGCTAGAAGAACAAAAGTGGCAGAGAAAAATAGAT
GAGACCCTTGAAAGACTCCGGGAACTTCAAGAGGCCACGGATGAGCTGGACCTCAAGCTGCGCCAAGCTGAGGTGAT
CAAGGGATCCTGGCAGCCCGTGGGCGATCTCCTCATTGACTCTCTCCAAGATCACCTCGAGAAAGTCAAGGCACTTC
GAGGAGAAATTGCGCCTCTGAAAGAGAACGTGAGCCACGTCAATGACCTTGCTCGCCAGCTTACCACTTTGGGCATT
CAGCTCTCACCGTATAACCTCAGCACTCTGGAAGACCTGAACACCAGATGGAAGCTTCTGCAGGTGGCCGTCGAGGA
CCGAGTCAGGCAGCTGCATGAAGCCCACAGGGACTTTGGTCCAGCATCTCAGCACTTTCTTTCCACGTCTGTCCAGG
GTCCCTGGGAGAGAGCCATCTCGCCAAACAAAGTGCCCTACTATATCAACCACGAGACTCAAACAACTTGCTGGGAC
CATCCCAAAATGACAGAGCTCTACCAGTCTTTAGCTGACCTGAATAATGTCAGATTCTCAGCTTATAGGACTGCCAT
GAAACTCCGAAGACTGCAGAAGGCCCTTTGCTTGGATCTCTTGAGCCTGTCAGCTGCATGTGATGCCTTGGACCAGC
ACAACCTCAAGCAAAATGACCAGCCCATGGATATCCTGCAGATTATTAATTGTTTGACCACTATTTATGACCGCCTG
GAGCAAGAGCACAACAATTTGGTCAACGTCCCTCTCTGCGTGGATATGTGTCTGAACTGGCTGCTGAATGTTTATGA
TACGGGACGAACAGGGAGGATCCGTGTCCTGTCTTTTAAAACTGGCATCATTTCCCTGTGTAAAGCACATTTGGAAG
ACAAGTACAGATACCTTTTCAAGCAAGTGGCAAGTTCAACAGGATTTTGTGACCAGCGCAGGCTGGGCCTCCTTCTG
CATGATTCTATCCAAATTCCAAGACAGTTGGGTGAAGTTGCATCCTTTGGGGGCAGTAACATTGAGCCAAGTGTCCG
GAGCTGCTTCCAATTTGCTAATAATAAGCCAGAGATCGAAGCGGCCCTCTTCCTAGACTGGATGAGACTGGAACCCC
AGTCCATGGTGTGGCTGCCCGTCCTGCACAGAGTGGCTGCTGCAGAAACTGCCAAGCATCAGGCCAAATGTAACATC
TGCAAAGAGTGTCCAATCATTGGATTCAGGTACAGGAGTCTAAAGCACTTTAATTATGACATCTGCCAAAGCTGCTT
TTTTTCTGGTCGAGTTGCAAAAGGCCATAAAATGCACTATCCCATGGTGGAATATTGCACTCCGACTACATCAGGAG
AAGATGTTCGAGACTTTGCCAAGGTACTAAAAAACAAATTTCGAACCAAAAGGTATTTTGCGAAGCATCCCCGAATG
GGCTACCTGCCAGTGCAGACTGTCTTAGAGGGGGACAACATGGAAACTCCCGTTACTCTGATCAACTTCTGGCCAGT
AGATTCTGCGCCTGCCTCGTCCCCTCAGCTTTCACACGATGATACTCATTCACGCATTGAACATTATGCTAGCAGGC
TAGCAGAAATGGAAAACAGCAATGGATCTTATCTAAATGATAGCATCTCTCCTAATGAGAGCATAGATGATGAACAT
TTGTTAATCCAGCATTACTGCCAAAGTTTGAACCAGGACTCCCCCCTGAGCCAGCCTCGTAGTCCTGCCCAGATCTT
GATTTCCTTAGAGAGTGAGGAAAGAGGGGAGCTAGAGAGAATCCTAGCAGATCTTGAGGAAGAAAACAGGAATCTGC
AAGCAGAATATGACCGTCTAAAGCAGCAGCACGAACATAAAGGCCTGTCCCCACTGCCGTCCCCTCCTGAAATGATG
CCCACCTCTCCCCAGAGTCCCCGGGATGCTGAGCTCATTGCTGAGGCCAAGCTACTGCGTCAACACAAAGGCCGCCT
GGAAGCCAGGATGCAAATCCTGGAAGACCACAATAAACAGCTGGAGTCACAGTTACACAGGCTAAGGCAGCTGCTGG
AGCAACCCCAGGCAGAGGCCAAAGTGAATGGCACAACGGTGTCCTCTCCTTCTACCTCTCTACAGAGGTCCGACAGC
AGTCAGCCTATGCTGCTCCGAGTGGTTGGCAGTCAAACTTCGGACTCCATGGGTGAGGAAGATCTTCTCAGTCCTCC
CCAGGACACAAGCACAGGGTTAGAGGAGGTGATGGAGCAACTCAACAACTCCTTCCCTAGTTCAAGAGGAAGAAATA
CCCCTGGAAAGCCAATGAGAGAGGACACAATG
Nterm-R2_R16-R17_R24 to end of dystrophin:
(SEQ ID NO: 29)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGTGGCAGAGAAA
AATAGATGAGACCCTTGAAAGACTCCGGGAACTTCAAGAGGCCACGGATGAGCTGGACCTCAAGCTGCGCCAAGCTG
AGGTGATCAAGGGATCCTGGCAGCCCGTGGGCGATCTCCTCATTGACTCTCTCCAAGATCACCTCGAGAAAGTCAAG
GCACTTCGAGGAGAAATTGCGCCTCTGAAAGAGAACGTGAGCCACGTCAATGACCTTGCTCGCCAGCTTACCACTTT
GGGCATTCAGCTCTCACCGTATAACCTCAGCACTCTGGAAGACCTGAACACCAGATGGAAGCTTCTGCAGGTGGCCG
TCGAGGACCGAGTCAGGCAGCTGCATGAAGCCCACAGGGACTTTGGTCCAGCATCTCAGCACTTTCTTTCCACGTCT
GTCCAGGGTCCCTGGGAGAGAGCCATCTCGCCAAACAAAGTGCCCTACTATATCAACCACGAGACTCAAACAACTTG
CTGGGACCATCCCAAAATGACAGAGCTCTACCAGTCTTTAGCTGACCTGAATAATGTCAGATTCTCAGCTTATAGGA
CTGCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTATGA
CCGCCTGGAGCAAGAGCACAACAATTTGGTCAACGTCCCTCTCTGCGTGGATATGTGTCTGAACTGGCTGCTGAATG
TTTATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGCCT
CCTTCTGCATGATTCTATCCAAATTCCAAGACAGTTGGGTGAAGTTGCATCCTTTGGGGGCAGTAACATTGAGCCAA
GTGTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAATG
TAACATCTGCAAAGAGTGTCCAATCATTGGATTCAGGTACAGGAGTCTAAAGCACTTTAATTATGACATCTGCCAAA
GCTGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCATCC
CCGAATGGGCTACCTGCCAGTGCAGACTGTCTTAGAGGGGGACAACATGGAAACTCCCGTTACTCTGATCAACTTCT
GGCCAGTAGATTCTGCGCCTGCCTCGTCCCCTCAGCTTTCACACGATGATACTCATTCACGCATTGAACATTATGCT
AGCAGGCTAGCAGAAATGGAAAACAGCAATGGATCTTATCTAAATGATAGCATCTCTCCTAATGAGAGCATAGATGA
TGAACATTTGTTAATCCAGCATTACTGCCAAAGTTTGAACCAGGACTCCCCCCTGAGCCAGCCTCGTAGTCCTGCCC
AGATCTTGATTTCCTTAGAGAGTGAGGAAAGAGGGGAGCTAGAGAGAATCCTAGCAGATCTTGAGGAAGAAAACAGG
AATCTGCAAGCAGAATATGACCGTCTAAAGCAGCAGCACGAACATAAAGGCCTGTCCCCACTGCCGTCCCCTCCTGA
AATGATGCCCACCTCTCCCCAGAGTCCCCGGGATGCTGAGCTCATTGCTGAGGCCAAGCTACTGCGTCAACACAAAG
GCCGCCTGGAAGCCAGGATGCAAATCCTGGAAGACCACAATAAACAGCTGGAGTCACAGTTACACAGGCTAAGGCAG
CTGCTGGAGCAACCCCAGGCAGAGGCCAAAGTGAATGGCACAACGGTGTCCTCTCCTTCTACCTCTCTACAGAGGTC
CGACAGCAGTCAGCCTATGCTGCTCCGAGTGGTTGGCAGTCAAACTTCGGACTCCATGGGTGAGGAAGATCTTCTCA
GTCCTCCCCAGGACACAAGCACAGGGTTAGAGGAGGTGATGGAGCAACTCAACAACTCCTTCCCTAGTTCAAGAGGA
AGAAATACCCCTGGAAAGCCAATGAGAGAGGACACAATG
Nterm-R2_R16-R17_R24 to end of syntrophin region:
(SEQ ID NO: 30)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCACACTGTCCGTGAAGAAACGATGAT
GGTGATGACTGAAGACATGCCTTTGGAAATTTCTTATGTGCCTTCTACTTATTTGACTGAAATCACTCATGTCTCAC
AAGCCCTATTAGAAGTGGAACAACTTCTCAATGCTCCTGACCTCTGTGCTAAGGACTTTGAAGATCTCTTTAAGCAA
GAGGAGTCTCTGAAGAATATAAAAGATAGTCTACAACAAAGCTCAGGTCGGATTGACATTATTCATAGCAAGAAGAC
AGCAGCATTGCAAAGTGCAACGCCTGTGGAAAGGGTGAAGCTACAGGAAGCTCTCTCCCAGCTTGATTTCCAATGGG
AAAAAGTTAACAAAATGTACAAGGACCGACAAGGGCGATTTGACAGATCTGTTGAGAAATGGCGGCGTTTTCATTAT
GATATAAAGATATTTAATCAGTGGCTAACAGAAGCTGAACAGTTTCTCAGAAAGACACAAATTCCTGAGAATTGGGA
ACATGCTAAATACAAATGGTATCTTAAGGAACTCCAGGATGGCATTGGGCAGCGGCAAACTGTTGTCAGAACATTGA
ATGCAACTGGGGAAGAAATAATTCAGCAATCCTCAAAAACAGATGCCAGTATTCTACAGGAAAAATTGGGAAGCCTG
AATCTGCGGTGGCAGGAGGTCTGCAAACAGCTGTCAGACAGAAAAAAGAGGCTAGAAGAACAAAAGTGGCAGAGAAA
AATAGATGAGACCCTTGAAAGACTCCGGGAACTTCAAGAGGCCACGGATGAGCTGGACCTCAAGCTGCGCCAAGCTG
AGGTGATCAAGGGATCCTGGCAGCCCGTGGGCGATCTCCTCATTGACTCTCTCCAAGATCACCTCGAGAAAGTCAAG
GCACTTCGAGGAGAAATTGCGCCTCTGAAAGAGAACGTGAGCCACGTCAATGACCTTGCTCGCCAGCTTACCACTTT
GGGCATTCAGCTCTCACCGTATAACCTCAGCACTCTGGAAGACCTGAACACCAGATGGAAGCTTCTGCAGGTGGCCG
TCGAGGACCGAGTCAGGCAGCTGCATGAAGCCCACAGGGACTTTGGTCCAGCATCTCAGCACTTTCTTTCCACGTCT
GTCCAGGGTCCCTGGGAGAGAGCCATCTCGCCAAACAAAGTGCCCTACTATATCAACCACGAGACTCAAACAACTTG
CTGGGACCATCCCAAAATGACAGAGCTCTACCAGTCTTTAGCTGACCTGAATAATGTCAGATTCTCAGCTTATAGGA
CTGCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTATGA
CCGCCTGGAGCAAGAGCACAACAATTTGGTCAACGTCCCTCTCTGCGTGGATATGTGTCTGAACTGGCTGCTGAATG
TTTATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGCCT
CCTTCTGCATGATTCTATCCAAATTCCAAGACAGTTGGGTGAAGTTGCATCCTTTGGGGGCAGTAACATTGAGCCAA
GTGTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAATG
TAACATCTGCAAAGAGTGTCCAATCATTGGATTCAGGTACAGGAGTCTAAAGCACTTTAATTATGACATCTGCCAAA
GCTGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCATCC
CCGAATGGGCTACCTGCCAGTGCAGACTGTCTTAGAGGGGGACAACATGGAAACTCCCGTTACTCTGATCAACTTCT
GGCCAGTAGATTCTGCGCCTGCCTCGTCCCCTCAGCTTTCACACGATGATACTCATTCACGCATTGAACATTATGCT
AGCAGGCTAGCAGAAATGGAAAACAGCAATGGATCTTATCTAAATGATAGCATCTCTCCTAATGAGAGCATAGATGA
TGAACATTTGTTAATCCAGCATTACTGCCAAAGTTTGAACCAGGACTCCCCCCTGAGCCAGCCTCGTAGT
Nterm-R2_R17_H3_R22_H4 to end of syntrophin region
(SEQ ID NO: 116)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCTGAAGACAAGTCATTTGGCAGTTCATT
GATGGAGAGTGAAGTAAACCTGGACCGTTATCAAACAGCTTTAGAAGAAGTATTATCGTGGCTTCTTTCTGCTGAGG
ACACATTGCAAGCACAAGGAGAGATTTCTAATGATGTGGAAGTGGTGAAAGACCAGTTTCATACTCATGAGGGGTAC
ATGATGGATTTGACAGCCCATCAGGGCCGGGTTGGTAATATTCTACAATTGGGAAGTAAGCTGATTGGAACAGGAAA
ATTATCAGAAGATGAAGAAACTGAAGTACAAGAGCAGATGAATCTCCTAAATTCAAGATGGGAATGCCTCAGGGTAG
CTAGCATGGAAAAACAAAGCAATTTACATAGAGTTTTAATGGATCTCCAGAATCAGAAACTGAAAGAGTTGAATGAC
TGGCTAACAAAAACAGAAGAAAGAACAAGGAAAATGGAGGAAGAGCCTCTTGGACCTGATCTTGAAGACCTAAAACG
CCAAGTACAACAACATAAGGTGCTTCAAGAAGATCTAGAACAAGAACAAGTCAGGGTCAATTCTCTCACTCACATGG
TGGTGGTAGTTGATGAATCTAGTGGAGATCACGCAACTGCTGCTTTGGAAGAACAACTTAAGGTATTGGGAGATCGA
TGGGCAAACATCTGTAGATGGACAGAAGACCGCTGGGTTCTTTTACAAGACCGATTTGACAGATCTGTTGAGAAATG
GCGGCGTTTTCATTATGATATAAAGATATTTAATCAGTGGCTAACAGAAGCTGAACAGTTTCTCAGAAAGACACAAA
TTCCTGAGAATTGGGAACATGCTAAATACAAATGGTATCTTAAGGAACTCCAGGATGGCATTGGGCAGCGGCAAACT
GTTGTCAGAACATTGAATGCAACTGGGGAAGAAATAATTCAGCAATCCTCAAAAACAGATGCCAGTATTCTACAGGA
AAAATTGGGAAGCCTGAATCTGCGGTGGCAGGAGGTCTGCAAACAGCTGTCAGACAGAAAAAAGAGGCTAGAAGAAC
AAAAGCAGCCTGACCTAGCTCCTGGACTGACCACTATTGGAGCCTCTCCTACTCAGACTGTTACTCTGGTGACACAA
CCTGTGGTTACTAAGGAAACTGCCATCTCCAAACTAGAAATGCCATCTTCCTTGATGTTGGAGGTACCTGAAGAAAC
TCATAGATTACTGCAACAGTTCCCCCTGGACCTGGAAAAGTTTCTTGCCTGGCTTACAGAAGCTGAAACAACTGCCA
ATGTCCTACAGGATGCTACCCGTAAGGAAAGGCTCCTAGAAGACTCCAAGGGAGTAAAAGAGCTGATGAAACAATGG
CAAGACCTCCAAGGTGAAATTGAAGCTCACACAGATGTTTATCACAACCTGGATGAAAACAGCCAAAAAATCCTGAG
ATCCCTGGAAGGTTCCGATGATGCAGTCCTGTTACAAAGACGTTTGGATAACATGAACTTCAAGTGGAGTGAACTTC
GGAAAAAGTCTCTCAACATTAGGTCCCATTTGGAAGCCAGTTCTGACCAGTGGAAGCGTCTGGGTCCAGCATCTCAG
CACTTTCTTTCCACGTCTGTCCAGGGTCCCTGGGAGAGAGCCATCTCGCCAAACAAAGTGCCCTACTATATCAACCA
CGAGACTCAAACAACTTGCTGGGACCATCCCAAAATGACAGAGCTCTACCAGTCTTTAGCTGACCTGAATAATGTCA
GATTCTCAGCTTATAGGACTGCCATGAAACTCCGAAGACTGCAGAAGGCCCTTTGCTTGGATCTCTTGAGCCTGTCA
GCTGCATGTGATGCCTTGGACCAGCACAACCTCAAGCAAAATGACCAGCCCATGGATATCCTGCAGATTATTAATTG
TTTGACCACTATTTATGACCGCCTGGAGCAAGAGCACAACAATTTGGTCAACGTCCCTCTCTGCGTGGATATGTGTC
TGAACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTGTGA
CCAGCGCAGGCTGGGCCTCCTTCTGCATGATTCTATCCAAATTCCAAGACAGTTGGGTGAAGTTGCATCCTTTGGGG
GCAGTAACATTGAGCCAAGTGTCCGGAGCTGCTTCCAATTTGCTAATAATAAGCCAGAGATCGAAGCGGCCCTCTTC
CTAGACTGGATGAGACTGGAACCCCAGTCCATGGTGTGGCTGCCCGTCCTGCACAGAGTGGCTGCTGCAGAAACTGC
CAAGCATCAGGCCAAATGTAACATCTGCAAAGAGTGTCCAATCATTGGATTCAGGTACAGGAGTCTAAAGCACTTTA
ATTATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAAAG
GTATTTTGCGAAGCATCCCCGAATGGGCTACCTGCCAGTGCAGACTGTCTTAGAGGGGGACAACATGGAAACTCCCG
TTACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCTCC
TAATGAGAGCATAGATGATGAACATTTGTTAATCCAGCATTACTGCCAAAGTTTGAACCAGGACTCCCCCCTGAGCC
AGCCTCGTAGT

Non-limiting examples of codon optimized nucleotide sequences encoding human micro-dystrophin proteins optimized for skeletal muscle are described below.

Nterm-R2_R16-R17_R24 to trCterm:
(SEQ ID NO: 31)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGTGGCAGCGGAA
GATCGACGAGACCCTGGAGCGGCTGCAGGAGCTGCAGGAGGCCACCGACGAGCTGGACCTGAAGCTGCGGCAGGCCG
AGGTGATCAAGGGCAGCTGGCAGCCCGTGGGCGACCTGCTGATCGACAGCCTGCAGGACCACCTGGAGAAGGTGAAG
GCCCTGCGGGGCGAGATCGCCCCCCTGAAGGAGAACGTGAGCCACGTGAACGACCTGGCCCGGCAGCTGACCACCCT
GGGCATCCAGCTGAGCCCCTACAACCTGAGCACCCTGGAGGACCTGAACACCCGGTGGAAGCTGCTGCAGGTGGCCG
TGGAGGACCGGGTGCGGCAGCTGCACGAGGCCCACCGGGACTTCGGCCCCGCCAGCCAGCACTTCCTGAGCACCAGC
GTGCAGGGCCCCTGGGAGCGGGCCATCAGCCCCAACAAGGTGCCCTACTACATCAACCACGAGACCCAGACCACCTG
CTGGGACCACCCCAAGATGACCGAGCTGTACCAGAGCCTGGCCGACCTGAACAACGTGCGGTTCAGCGCCTACCGGA
CCGCCATGAAGCTGCGGCGGCTGCAGAAGGCCCTGTGCCTGGACCTGCTGAGCCTGAGCGCCGCCTGCGACGCCCTG
GACCAGCACAACCTGAAGCAGAACGACCAGCCCATGGACATCCTGCAGATCATCAACTGCCTGACCACCATCTACGA
CCGGCTGGAGCAGGAGCACAACAACCTGGTGAACGTGCCCCTGTGCGTGGACATGTGCCTGAACTGGCTGCTGAACG
TGTACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGCCT
GCTGCTGCACGACAGCATCCAGATCCCCCGGCAGCTGGGCGAGGTGGCCAGCTTCGGCGGCAGCAACATCGAGCCCA
GCGTGCGGAGCTGCTTCCAGTTCGCCAACAACAAGCCCGAGATCGAGGCCGCCCTGTTCCTGGACTGGATGCGGCTG
GAGCCCCAGAGCATGGTGTGGCTGCCCGTGCTGCACCGGGTGGCCGCCGCCGAGACCGCCAAGCACCAGGCCAAGTG
CAACATCTGCAAGGAGTGCCCCATCATCGGCTTCCGGTACCGGAGCCTGAAGCACTTCAACTACGACATCTGCCAGA
GCTGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCACCC
CCGGATGGGCTACCTGCCCGTGCAGACCGTGCTGGAGGGCGACAACATGGAGACCCCC
Nterm-R2_R16-R17_H4 to end of syntrophin region:
(SEQ ID NO: 32)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGGGCCCCGCCAG
CCAGCACTTCCTGAGCACCAGCGTGCAGGGCCCCTGGGAGCGGGCCATCAGCCCCAACAAGGTGCCCTACTACATCA
ACCACGAGACCCAGACCACCTGCTGGGACCACCCCAAGATGACCGAGCTGTACCAGAGCCTGGCCGACCTGAACAAC
GTGCGGTTCAGCGCCTACCGGACCGCCATGAAGCTGCGGCGGCTGCAGAAGGCCCTGTGCCTGGACCTGCTGAGCCT
GAGCGCCGCCTGCGACGCCCTGGACCAGCACAACCTGAAGCAGAACGACCAGCCCATGGACATCCTGCAGATCATCA
ACTGCCTGACCACCATCTACGACCGGCTGGAGCAGGAGCACAACAACCTGGTGAACGTGCCCCTGTGCGTGGACATG
TGCCTGAACTGGCTGCTGAACGTGTACGACACCGGCCGGACCGGCCGGATCCGGGTGCTGAGCTTCAAGACCGGCAT
CATCAGCCTGTGCAAGGCCCACCTGGAGGACAAGTACCGGTACCTGTTCAAGCAGGTGGCCAGCAGCACCGGCTTCT
GCGACCAGCGGCGGCTGGGCCTGCTGCTGCACGACAGCATCCAGATCCCCCGGCAGCTGGGCGAGGTGGCCAGCTTC
GGCGGCAGCAACATCGAGCCCAGCGTGCGGAGCTGCTTCCAGTTCGCCAACAACAAGCCCGAGATCGAGGCCGCCCT
GTTCCTGGACTGGATGCGGCTGGAGCCCCAGAGCATGGTGTGGCTGCCCGTGCTGCACCGGGTGGCCGCCGCCGAGA
CCGCCAAGCACCAGGCCAAGTGCAACATCTGCAAGGAGTGCCCCATCATCGGCTTCCGGTACCGGAGCCTGAAGCAC
TTCAACTACGACATCTGCCAGAGCTGCTTCTTCAGCGGCCGGGTGGCCAAGGGCCACAAGATGCACTACCCCATGGT
GGAGTACTGCACCCCCACCACCAGCGGCGAGGACGTGCGGGACTTCGCCAAGGTGCTGAAGAACAAGTTCCGGACCA
AGCGGTACTTCGCCAAGCACCCCCGGATGGGCTACCTGCCCGTGCAGACCGTGCTGGAGGGCGACAACATGGAGACC
CCCGTGACCCTGATCAACTTCTGGCCCGTGGACAGCGCCCCCGCCAGCAGCCCCCAGCTGAGCCACGACGACACCCA
CAGCCGGATCGAGCACTACGCCAGCCGGCTGGCCGAGATGGAGAACAGCAACGGCAGCTACCTGAACGACAGCATCA
GCCCCAACGAGAGCATCGACGACGAGCACCTGCTGATCCAGCACTACTGCCAGAGCCTGAACCAGGACAGCCCCCTG
AGCCAGCCCCGGAGC
Nterm-R2_R16-R17_H4 to 1st coiled coil and proline rich region that follows:
(SEQ ID NO: 33)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGGGCCCCGCCAG
CCAGCACTTCCTGAGCACCAGCGTGCAGGGCCCCTGGGAGCGGGCCATCAGCCCCAACAAGGTGCCCTACTACATCA
ACCACGAGACCCAGACCACCTGCTGGGACCACCCCAAGATGACCGAGCTGTACCAGAGCCTGGCCGACCTGAACAAC
GTGCGGTTCAGCGCCTACCGGACCGCCATGAAGCTGCGGCGGCTGCAGAAGGCCCTGTGCCTGGACCTGCTGAGCCT
GAGCGCCGCCTGCGACGCCCTGGACCAGCACAACCTGAAGCAGAACGACCAGCCCATGGACATCCTGCAGATCATCA
ACTGCCTGACCACCATCTACGACCGGCTGGAGCAGGAGCACAACAACCTGGTGAACGTGCCCCTGTGCGTGGACATG
TGCCTGAACTGGCTGCTGAACGTGTACGACACCGGCCGGACCGGCCGGATCCGGGTGCTGAGCTTCAAGACCGGCAT
CATCAGCCTGTGCAAGGCCCACCTGGAGGACAAGTACCGGTACCTGTTCAAGCAGGTGGCCAGCAGCACCGGCTTCT
GCGACCAGCGGCGGCTGGGCCTGCTGCTGCACGACAGCATCCAGATCCCCCGGCAGCTGGGCGAGGTGGCCAGCTTC
GGCGGCAGCAACATCGAGCCCAGCGTGCGGAGCTGCTTCCAGTTCGCCAACAACAAGCCCGAGATCGAGGCCGCCCT
GTTCCTGGACTGGATGCGGCTGGAGCCCCAGAGCATGGTGTGGCTGCCCGTGCTGCACCGGGTGGCCGCCGCCGAGA
CCGCCAAGCACCAGGCCAAGTGCAACATCTGCAAGGAGTGCCCCATCATCGGCTTCCGGTACCGGAGCCTGAAGCAC
TTCAACTACGACATCTGCCAGAGCTGCTTCTTCAGCGGCCGGGTGGCCAAGGGCCACAAGATGCACTACCCCATGGT
GGAGTACTGCACCCCCACCACCAGCGGCGAGGACGTGCGGGACTTCGCCAAGGTGCTGAAGAACAAGTTCCGGACCA
AGCGGTACTTCGCCAAGCACCCCCGGATGGGCTACCTGCCCGTGCAGACCGTGCTGGAGGGCGACAACATGGAGACC
CCCGTGACCCTGATCAACTTCTGGCCCGTGGACAGCGCCCCCGCCAGCAGCCCCCAGCTGAGCCACGACGACACCCA
CAGCCGGATCGAGCACTACGCCAGCCGGCTGGCCGAGATGGAGAACAGCAACGGCAGCTACCTGAACGACAGCATCA
GCCCCAACGAGAGCATCGACGACGAGCACCTGCTGATCCAGCACTACTGCCAGAGCCTGAACCAGGACAGCCCCCTG
AGCCAGCCCCGGAGCCCCGCCCAGATCCTGATCAGCCTGGAGAGCGAGGAGCGGGGCGAGCTGGAGCGGATCCTGGC
CGACCTGGAGGAGGAGAACCGGAACCTGCAGGCCGAGTACGACCGGCTGAAGCAGCAGCACGAGCACAAGGGCCTGA
GCCCCCTGCCCAGCCCCCCCGAGATGATGCCCACCAGCCCCCAGAGCCCC
Nterm-R2_R16-R17_R24 to 1st coiled coil and proline rich region that follows:
(SEQ ID NO: 34)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGTGGCAGCGGAA
GATCGACGAGACCCTGGAGCGGCTGCAGGAGCTGCAGGAGGCCACCGACGAGCTGGACCTGAAGCTGCGGCAGGCCG
AGGTGATCAAGGGCAGCTGGCAGCCCGTGGGCGACCTGCTGATCGACAGCCTGCAGGACCACCTGGAGAAGGTGAAG
GCCCTGCGGGGCGAGATCGCCCCCCTGAAGGAGAACGTGAGCCACGTGAACGACCTGGCCCGGCAGCTGACCACCCT
GGGCATCCAGCTGAGCCCCTACAACCTGAGCACCCTGGAGGACCTGAACACCCGGTGGAAGCTGCTGCAGGTGGCCG
TGGAGGACCGGGTGCGGCAGCTGCACGAGGCCCACCGGGACTTCGGCCCCGCCAGCCAGCACTTCCTGAGCACCAGC
GTGCAGGGCCCCTGGGAGCGGGCCATCAGCCCCAACAAGGTGCCCTACTACATCAACCACGAGACCCAGACCACCTG
CTGGGACCACCCCAAGATGACCGAGCTGTACCAGAGCCTGGCCGACCTGAACAACGTGCGGTTCAGCGCCTACCGGA
CCGCCATGAAGCTGCGGCGGCTGCAGAAGGCCCTGTGCCTGGACCTGCTGAGCCTGAGCGCCGCCTGCGACGCCCTG
GACCAGCACAACCTGAAGCAGAACGACCAGCCCATGGACATCCTGCAGATCATCAACTGCCTGACCACCATCTACGA
CCGGCTGGAGCAGGAGCACAACAACCTGGTGAACGTGCCCCTGTGCGTGGACATGTGCCTGAACTGGCTGCTGAACG
TGTACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGCCT
GCTGCTGCACGACAGCATCCAGATCCCCCGGCAGCTGGGCGAGGTGGCCAGCTTCGGCGGCAGCAACATCGAGCCCA
GCGTGCGGAGCTGCTTCCAGTTCGCCAACAACAAGCCCGAGATCGAGGCCGCCCTGTTCCTGGACTGGATGCGGCTG
GAGCCCCAGAGCATGGTGTGGCTGCCCGTGCTGCACCGGGTGGCCGCCGCCGAGACCGCCAAGCACCAGGCCAAGTG
CAACATCTGCAAGGAGTGCCCCATCATCGGCTTCCGGTACCGGAGCCTGAAGCACTTCAACTACGACATCTGCCAGA
GCTGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCACCC
CCGGATGGGCTACCTGCCCGTGCAGACCGTGCTGGAGGGCGACAACATGGAGACCCCCGTGACCCTGATCAACTTCT
GGCCCGTGGACAGCGCCCCCGCCAGCAGCCCCCAGCTGAGCCACGACGACACCCACAGCCGGATCGAGCACTACGCC
AGCCGGCTGGCCGAGATGGAGAACAGCAACGGCAGCTACCTGAACGACAGCATCAGCCCCAACGAGAGCATCGACGA
CGAGCACCTGCTGATCCAGCACTACTGCCAGAGCCTGAACCAGGACAGCCCCCTGAGCCAGCCCCGGAGCCCCGCCC
AGATCCTGATCAGCCTGGAGAGCGAGGAGCGGGGCGAGCTGGAGCGGATCCTGGCCGACCTGGAGGAGGAGAACCGG
AACCTGCAGGCCGAGTACGACCGGCTGAAGCAGCAGCACGAGCACAAGGGCCTGAGCCCCCTGCCCAGCCCCCCCGA
GATGATGCCCACCAGCCCCCAGAGCCCC
Nterm-R2_R23_R24 to end of dystrophin:
(SEQ ID NO: 35)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACCTGAGCCTGCAGGAGCTGCTGGT
GTGGCTGCAGCTGAAGGACGACGAGCTGAGCCGGCAGGCCCCCATCGGCGGCGACTTCCCCGCCGTGCAGAAGCAGA
ACGACGTGCACCGGGCCTTCAAGCGGGAGCTGAAGACCAAGGAGCCCGTGATCATGAGCACCCTGGAGACCGTGCGG
ATCTTCCTGACCGAGCAGCCCCTGGAGGGCCTGGAGAAGCTGTACCAGGAGCCCCGGGAGCTGCCCCCCGAGGAGCG
GGCCCAGAACGTGACCCGGCTGCTGCGGAAGCAGGCCGAGGAGGTGAACACCGAGTGGGAGAAGCTGAACCTGCACA
GCGCCGACTGGCAGCGGAAGATCGACGAGACCCTGGAGCGGCTGCAGGAGCTGCAGGAGGCCACCGACGAGCTGGAC
CTGAAGCTGCGGCAGGCCGAGGTGATCAAGGGCAGCTGGCAGCCCGTGGGCGACCTGCTGATCGACAGCCTGCAGGA
CCACCTGGAGAAGGTGAAGGCCCTGCGGGGCGAGATCGCCCCCCTGAAGGAGAACGTGAGCCACGTGAACGACCTGG
CCCGGCAGCTGACCACCCTGGGCATCCAGCTGAGCCCCTACAACCTGAGCACCCTGGAGGACCTGAACACCCGGTGG
AAGCTGCTGCAGGTGGCCGTGGAGGACCGGGTGCGGCAGCTGCACGAGGCCCACCGGGACTTCGGCCCCGCCAGCCA
GCACTTCCTGAGCACCAGCGTGCAGGGCCCCTGGGAGCGGGCCATCAGCCCCAACAAGGTGCCCTACTACATCAACC
ACGAGACCCAGACCACCTGCTGGGACCACCCCAAGATGACCGAGCTGTACCAGAGCCTGGCCGACCTGAACAACGTG
CGGTTCAGCGCCTACCGGACCGCCATGAAGCTGCGGCGGCTGCAGAAGGCCCTGTGCCTGGACCTGCTGAGCCTGAG
CGCCGCCTGCGACGCCCTGGACCAGCACAACCTGAAGCAGAACGACCAGCCCATGGACATCCTGCAGATCATCAACT
GCCTGACCACCATCTACGACCGGCTGGAGCAGGAGCACAACAACCTGGTGAACGTGCCCCTGTGCGTGGACATGTGC
CTGAACTGGCTGCTGAACGTGTACGACACCGGCCGGACCGGCCGGATCCGGGTGCTGAGCTTCAAGACCGGCATCAT
CAGCCTGTGCAAGGCCCACCTGGAGGACAAGTACCGGTACCTGTTCAAGCAGGTGGCCAGCAGCACCGGCTTCTGCG
ACCAGCGGCGGCTGGGCCTGCTGCTGCACGACAGCATCCAGATCCCCCGGCAGCTGGGCGAGGTGGCCAGCTTCGGC
GGCAGCAACATCGAGCCCAGCGTGCGGAGCTGCTTCCAGTTCGCCAACAACAAGCCCGAGATCGAGGCCGCCCTGTT
CCTGGACTGGATGCGGCTGGAGCCCCAGAGCATGGTGTGGCTGCCCGTGCTGCACCGGGTGGCCGCCGCCGAGACCG
CCAAGCACCAGGCCAAGTGCAACATCTGCAAGGAGTGCCCCATCATCGGCTTCCGGTACCGGAGCCTGAAGCACTTC
AACTACGACATCTGCCAGAGCTGCTTCTTCAGCGGCCGGGTGGCCAAGGGCCACAAGATGCACTACCCCATGGTGGA
GTACTGCACCCCCACCACCAGCGGCGAGGACGTGCGGGACTTCGCCAAGGTGCTGAAGAACAAGTTCCGGACCAAGC
GGTACTTCGCCAAGCACCCCCGGATGGGCTACCTGCCCGTGCAGACCGTGCTGGAGGGCGACAACATGGAGACCCCC
GTGACCCTGATCAACTTCTGGCCCGTGGACAGCGCCCCCGCCAGCAGCCCCCAGCTGAGCCACGACGACACCCACAG
CCGGATCGAGCACTACGCCAGCCGGCTGGCCGAGATGGAGAACAGCAACGGCAGCTACCTGAACGACAGCATCAGCC
CCAACGAGAGCATCGACGACGAGCACCTGCTGATCCAGCACTACTGCCAGAGCCTGAACCAGGACAGCCCCCTGAGC
CAGCCCCGGAGCCCCGCCCAGATCCTGATCAGCCTGGAGAGCGAGGAGCGGGGCGAGCTGGAGCGGATCCTGGCCGA
CCTGGAGGAGGAGAACCGGAACCTGCAGGCCGAGTACGACCGGCTGAAGCAGCAGCACGAGCACAAGGGCCTGAGCC
CCCTGCCCAGCCCCCCCGAGATGATGCCCACCAGCCCCCAGAGCCCCCGGGACGCCGAGCTGATCGCCGAGGCCAAG
CTGCTGCGGCAGCACAAGGGCCGGCTGGAGGCCCGGATGCAGATCCTGGAGGACCACAACAAGCAGCTGGAGAGCCA
GCTGCACCGGCTGCGGCAGCTGCTGGAGCAGCCCCAGGCCGAGGCCAAGGTGAACGGCACCACCGTGAGCAGCCCCA
GCACCAGCCTGCAGCGGAGCGACAGCAGCCAGCCCATGCTGCTGCGGGTGGTGGGCAGCCAGACCAGCGACAGCATG
GGCGAGGAGGACCTGCTGAGCCCCCCCCAGGACACCAGCACCGGCCTGGAGGAGGTGATGGAGCAGCTGAACAACAG
CTTCCCCAGCAGCCGGGGCCGGAACACCCCCGGCAAGCCCATGCGGGAGGACACCATG
Nterm-R2_R23_R24 to end of dystrophin:
(SEQ ID NO: 118)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACGCCAGCAGCGACCAGTGGAAGCGGCT
GCACCTGAGCCTGCAGGAGCTGCTGGTGTGGCTGCAGCTGAAGGACGACGAGCTGAGCCGGCAGGCCCCCATCGGCG
GCGACTTCCCCGCCGTGCAGAAGCAGAACGACGTGCACCGGGCCTTCAAGCGGGAGCTGAAGACCAAGGAGCCCGTG
ATCATGAGCACCCTGGAGACCGTGCGGATCTTCCTGACCGAGCAGCCCCTGGAGGGCCTGGAGAAGCTGTACCAGGA
GCCCCGGGAGCTGCCCCCCGAGGAGCGGGCCCAGAACGTGACCCGGCTGCTGCGGAAGCAGGCCGAGGAGGTGAACA
CCGAGTGGGAGAAGCTGAACCTGCACAGCGCCGACTGGCAGCGGAAGATCGACGAGACCCTGGAGCGGCTGCAGGAG
CTGCAGGAGGCCACCGACGAGCTGGACCTGAAGCTGCGGCAGGCCGAGGTGATCAAGGGCAGCTGGCAGCCCGTGGG
CGACCTGCTGATCGACAGCCTGCAGGACCACCTGGAGAAGGTGAAGGCCCTGCGGGGCGAGATCGCCCCCCTGAAGG
AGAACGTGAGCCACGTGAACGACCTGGCCCGGCAGCTGACCACCCTGGGCATCCAGCTGAGCCCCTACAACCTGAGC
ACCCTGGAGGACCTGAACACCCGGTGGAAGCTGCTGCAGGTGGCCGTGGAGGACCGGGTGCGGCAGCTGCACGAGGC
CCACCGGGACTTCGGCCCCGCCAGCCAGCACTTCCTGAGCACCAGCGTGCAGGGCCCCTGGGAGCGGGCCATCAGCC
CCAACAAGGTGCCCTACTACATCAACCACGAGACCCAGACCACCTGCTGGGACCACCCCAAGATGACCGAGCTGTAC
CAGAGCCTGGCCGACCTGAACAACGTGCGGTTCAGCGCCTACCGGACCGCCATGAAGCTGCGGCGGCTGCAGAAGGC
CCTGTGCCTGGACCTGCTGAGCCTGAGCGCCGCCTGCGACGCCCTGGACCAGCACAACCTGAAGCAGAACGACCAGC
CCATGGACATCCTGCAGATCATCAACTGCCTGACCACCATCTACGACCGGCTGGAGCAGGAGCACAACAACCTGGTG
AACGTGCCCCTGTGCGTGGACATGTGCCTGAACTGGCTGCTGAACGTGTACGACACCGGCCGGACCGGCCGGATCCG
GGTGCTGAGCTTCAAGACCGGCATCATCAGCCTGTGCAAGGCCCACCTGGAGGACAAGTACCGGTACCTGTTCAAGC
AGGTGGCCAGCAGCACCGGCTTCTGCGACCAGCGGCGGCTGGGCCTGCTGCTGCACGACAGCATCCAGATCCCCCGG
CAGCTGGGCGAGGTGGCCAGCTTCGGCGGCAGCAACATCGAGCCCAGCGTGCGGAGCTGCTTCCAGTTCGCCAACAA
CAAGCCCGAGATCGAGGCCGCCCTGTTCCTGGACTGGATGCGGCTGGAGCCCCAGAGCATGGTGTGGCTGCCCGTGC
TGCACCGGGTGGCCGCCGCCGAGACCGCCAAGCACCAGGCCAAGTGCAACATCTGCAAGGAGTGCCCCATCATCGGC
TTCCGGTACCGGAGCCTGAAGCACTTCAACTACGACATCTGCCAGAGCTGCTTCTTCAGCGGCCGGGTGGCCAAGGG
CCACAAGATGCACTACCCCATGGTGGAGTACTGCACCCCCACCACCAGCGGCGAGGACGTGCGGGACTTCGCCAAGG
TGCTGAAGAACAAGTTCCGGACCAAGCGGTACTTCGCCAAGCACCCCCGGATGGGCTACCTGCCCGTGCAGACCGTG
CTGGAGGGCGACAACATGGAGACCCCCGTGACCCTGATCAACTTCTGGCCCGTGGACAGCGCCCCCGCCAGCAGCCC
CCAGCTGAGCCACGACGACACCCACAGCCGGATCGAGCACTACGCCAGCCGGCTGGCCGAGATGGAGAACAGCAACG
GCAGCTACCTGAACGACAGCATCAGCCCCAACGAGAGCATCGACGACGAGCACCTGCTGATCCAGCACTACTGCCAG
AGCCTGAACCAGGACAGCCCCCTGAGCCAGCCCCGGAGCCCCGCCCAGATCCTGATCAGCCTGGAGAGCGAGGAGCG
GGGCGAGCTGGAGCGGATCCTGGCCGACCTGGAGGAGGAGAACCGGAACCTGCAGGCCGAGTACGACCGGCTGAAGC
AGCAGCACGAGCACAAGGGCCTGAGCCCCCTGCCCAGCCCCCCCGAGATGATGCCCACCAGCCCCCAGAGCCCCCGG
GACGCCGAGCTGATCGCCGAGGCCAAGCTGCTGCGGCAGCACAAGGGCCGGCTGGAGGCCCGGATGCAGATCCTGGA
GGACCACAACAAGCAGCTGGAGAGCCAGCTGCACCGGCTGCGGCAGCTGCTGGAGCAGCCCCAGGCCGAGGCCAAGG
TGAACGGCACCACCGTGAGCAGCCCCAGCACCAGCCTGCAGCGGAGCGACAGCAGCCAGCCCATGCTGCTGCGGGTG
GTGGGCAGCCAGACCAGCGACAGCATGGGCGAGGAGGACCTGCTGAGCCCCCCCCAGGACACCAGCACCGGCCTGGA
GGAGGTGATGGAGCAGCTGAACAACAGCTTCCCCAGCAGCCGGGGCCGGAACACCCCCGGCAAGCCCATGCGGGAGG
ACACCATG
Nterm-R2_R16-R17_H4 to end of dystrophin:
(SEQ ID NO: 36)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGGGCCCCGCCAG
CCAGCACTTCCTGAGCACCAGCGTGCAGGGCCCCTGGGAGCGGGCCATCAGCCCCAACAAGGTGCCCTACTACATCA
ACCACGAGACCCAGACCACCTGCTGGGACCACCCCAAGATGACCGAGCTGTACCAGAGCCTGGCCGACCTGAACAAC
GTGCGGTTCAGCGCCTACCGGACCGCCATGAAGCTGCGGCGGCTGCAGAAGGCCCTGTGCCTGGACCTGCTGAGCCT
GAGCGCCGCCTGCGACGCCCTGGACCAGCACAACCTGAAGCAGAACGACCAGCCCATGGACATCCTGCAGATCATCA
ACTGCCTGACCACCATCTACGACCGGCTGGAGCAGGAGCACAACAACCTGGTGAACGTGCCCCTGTGCGTGGACATG
TGCCTGAACTGGCTGCTGAACGTGTACGACACCGGCCGGACCGGCCGGATCCGGGTGCTGAGCTTCAAGACCGGCAT
CATCAGCCTGTGCAAGGCCCACCTGGAGGACAAGTACCGGTACCTGTTCAAGCAGGTGGCCAGCAGCACCGGCTTCT
GCGACCAGCGGCGGCTGGGCCTGCTGCTGCACGACAGCATCCAGATCCCCCGGCAGCTGGGCGAGGTGGCCAGCTTC
GGCGGCAGCAACATCGAGCCCAGCGTGCGGAGCTGCTTCCAGTTCGCCAACAACAAGCCCGAGATCGAGGCCGCCCT
GTTCCTGGACTGGATGCGGCTGGAGCCCCAGAGCATGGTGTGGCTGCCCGTGCTGCACCGGGTGGCCGCCGCCGAGA
CCGCCAAGCACCAGGCCAAGTGCAACATCTGCAAGGAGTGCCCCATCATCGGCTTCCGGTACCGGAGCCTGAAGCAC
TTCAACTACGACATCTGCCAGAGCTGCTTCTTCAGCGGCCGGGTGGCCAAGGGCCACAAGATGCACTACCCCATGGT
GGAGTACTGCACCCCCACCACCAGCGGCGAGGACGTGCGGGACTTCGCCAAGGTGCTGAAGAACAAGTTCCGGACCA
AGCGGTACTTCGCCAAGCACCCCCGGATGGGCTACCTGCCCGTGCAGACCGTGCTGGAGGGCGACAACATGGAGACC
CCCGTGACCCTGATCAACTTCTGGCCCGTGGACAGCGCCCCCGCCAGCAGCCCCCAGCTGAGCCACGACGACACCCA
CAGCCGGATCGAGCACTACGCCAGCCGGCTGGCCGAGATGGAGAACAGCAACGGCAGCTACCTGAACGACAGCATCA
GCCCCAACGAGAGCATCGACGACGAGCACCTGCTGATCCAGCACTACTGCCAGAGCCTGAACCAGGACAGCCCCCTG
AGCCAGCCCCGGAGCCCCGCCCAGATCCTGATCAGCCTGGAGAGCGAGGAGCGGGGCGAGCTGGAGCGGATCCTGGC
CGACCTGGAGGAGGAGAACCGGAACCTGCAGGCCGAGTACGACCGGCTGAAGCAGCAGCACGAGCACAAGGGCCTGA
GCCCCCTGCCCAGCCCCCCCGAGATGATGCCCACCAGCCCCCAGAGCCCCCGGGACGCCGAGCTGATCGCCGAGGCC
AAGCTGCTGCGGCAGCACAAGGGCCGGCTGGAGGCCCGGATGCAGATCCTGGAGGACCACAACAAGCAGCTGGAGAG
CCAGCTGCACCGGCTGCGGCAGCTGCTGGAGCAGCCCCAGGCCGAGGCCAAGGTGAACGGCACCACCGTGAGCAGCC
CCAGCACCAGCCTGCAGCGGAGCGACAGCAGCCAGCCCATGCTGCTGCGGGTGGTGGGCAGCCAGACCAGCGACAGC
ATGGGCGAGGAGGACCTGCTGAGCCCCCCCCAGGACACCAGCACCGGCCTGGAGGAGGTGATGGAGCAGCTGAACAA
CAGCTTCCCCAGCAGCCGGGGCCGGAACACCCCCGGCAAGCCCATGCGGGAGGACACCATG
Nterm-R1_R16-R17_R24 to end of dystrophin:
(SEQ ID NO: 37)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCACACCGTGCGGGAGGAGACCATGATGGTGATG
ACCGAGGACATGCCCCTGGAGATCAGCTACGTGCCCAGCACCTACCTGACCGAGATCACCCACGTGAGCCAGGCCCT
GCTGGAGGTGGAGCAGCTGCTGAACGCCCCCGACCTGTGCGCCAAGGACTTCGAGGACCTGTTCAAGCAGGAGGAGA
GCCTGAAGAACATCAAGGACAGCCTGCAGCAGAGCAGCGGCCGGATCGACATCATCCACAGCAAGAAGACCGCCGCC
CTGCAGAGCGCCACCCCCGTGGAGCGGGTGAAGCTGCAGGAGGCCCTGAGCCAGCTGGACTTCCAGTGGGAGAAGGT
GAACAAGATGTACAAGGACCGGCAGGGCCGGTTCGACCGGAGCGTGGAGAAGTGGCGGCGGTTCCACTACGACATCA
AGATCTTCAACCAGTGGCTGACCGAGGCCGAGCAGTTCCTGCGGAAGACCCAGATCCCCGAGAACTGGGAGCACGCC
AAGTACAAGTGGTACCTGAAGGAGCTGCAGGACGGCATCGGCCAGCGGCAGACCGTGGTGCGGACCCTGAACGCCAC
CGGCGAGGAGATCATCCAGCAGAGCAGCAAGACCGACGCCAGCATCCTGCAGGAGAAGCTGGGCAGCCTGAACCTGC
GGTGGCAGGAGGTGTGCAAGCAGCTGAGCGACCGGAAGAAGCGGCTGGAGGAGCAGAAGTGGCAGCGGAAGATCGAC
GAGACCCTGGAGCGGCTGCAGGAGCTGCAGGAGGCCACCGACGAGCTGGACCTGAAGCTGCGGCAGGCCGAGGTGAT
CAAGGGCAGCTGGCAGCCCGTGGGCGACCTGCTGATCGACAGCCTGCAGGACCACCTGGAGAAGGTGAAGGCCCTGC
GGGGCGAGATCGCCCCCCTGAAGGAGAACGTGAGCCACGTGAACGACCTGGCCCGGCAGCTGACCACCCTGGGCATC
CAGCTGAGCCCCTACAACCTGAGCACCCTGGAGGACCTGAACACCCGGTGGAAGCTGCTGCAGGTGGCCGTGGAGGA
CCGGGTGCGGCAGCTGCACGAGGCCCACCGGGACTTCGGCCCCGCCAGCCAGCACTTCCTGAGCACCAGCGTGCAGG
GCCCCTGGGAGCGGGCCATCAGCCCCAACAAGGTGCCCTACTACATCAACCACGAGACCCAGACCACCTGCTGGGAC
CACCCCAAGATGACCGAGCTGTACCAGAGCCTGGCCGACCTGAACAACGTGCGGTTCAGCGCCTACCGGACCGCCAT
GAAGCTGCGGCGGCTGCAGAAGGCCCTGTGCCTGGACCTGCTGAGCCTGAGCGCCGCCTGCGACGCCCTGGACCAGC
ACAACCTGAAGCAGAACGACCAGCCCATGGACATCCTGCAGATCATCAACTGCCTGACCACCATCTACGACCGGCTG
GAGCAGGAGCACAACAACCTGGTGAACGTGCCCCTGTGCGTGGACATGTGCCTGAACTGGCTGCTGAACGTGTACGA
CACCGGCCGGACCGGCCGGATCCGGGTGCTGAGCTTCAAGACCGGCATCATCAGCCTGTGCAAGGCCCACCTGGAGG
ACAAGTACCGGTACCTGTTCAAGCAGGTGGCCAGCAGCACCGGCTTCTGCGACCAGCGGCGGCTGGGCCTGCTGCTG
CACGACAGCATCCAGATCCCCCGGCAGCTGGGCGAGGTGGCCAGCTTCGGCGGCAGCAACATCGAGCCCAGCGTGCG
GAGCTGCTTCCAGTTCGCCAACAACAAGCCCGAGATCGAGGCCGCCCTGTTCCTGGACTGGATGCGGCTGGAGCCCC
AGAGCATGGTGTGGCTGCCCGTGCTGCACCGGGTGGCCGCCGCCGAGACCGCCAAGCACCAGGCCAAGTGCAACATC
TGCAAGGAGTGCCCCATCATCGGCTTCCGGTACCGGAGCCTGAAGCACTTCAACTACGACATCTGCCAGAGCTGCTT
CTTCAGCGGCCGGGTGGCCAAGGGCCACAAGATGCACTACCCCATGGTGGAGTACTGCACCCCCACCACCAGCGGCG
AGGACGTGCGGGACTTCGCCAAGGTGCTGAAGAACAAGTTCCGGACCAAGCGGTACTTCGCCAAGCACCCCCGGATG
GGCTACCTGCCCGTGCAGACCGTGCTGGAGGGCGACAACATGGAGACCCCCGTGACCCTGATCAACTTCTGGCCCGT
GGACAGCGCCCCCGCCAGCAGCCCCCAGCTGAGCCACGACGACACCCACAGCCGGATCGAGCACTACGCCAGCCGGC
TGGCCGAGATGGAGAACAGCAACGGCAGCTACCTGAACGACAGCATCAGCCCCAACGAGAGCATCGACGACGAGCAC
CTGCTGATCCAGCACTACTGCCAGAGCCTGAACCAGGACAGCCCCCTGAGCCAGCCCCGGAGCCCCGCCCAGATCCT
GATCAGCCTGGAGAGCGAGGAGCGGGGCGAGCTGGAGCGGATCCTGGCCGACCTGGAGGAGGAGAACCGGAACCTGC
AGGCCGAGTACGACCGGCTGAAGCAGCAGCACGAGCACAAGGGCCTGAGCCCCCTGCCCAGCCCCCCCGAGATGATG
CCCACCAGCCCCCAGAGCCCCCGGGACGCCGAGCTGATCGCCGAGGCCAAGCTGCTGCGGCAGCACAAGGGCCGGCT
GGAGGCCCGGATGCAGATCCTGGAGGACCACAACAAGCAGCTGGAGAGCCAGCTGCACCGGCTGCGGCAGCTGCTGG
AGCAGCCCCAGGCCGAGGCCAAGGTGAACGGCACCACCGTGAGCAGCCCCAGCACCAGCCTGCAGCGGAGCGACAGC
AGCCAGCCCATGCTGCTGCGGGTGGTGGGCAGCCAGACCAGCGACAGCATGGGCGAGGAGGACCTGCTGAGCCCCCC
CCAGGACACCAGCACCGGCCTGGAGGAGGTGATGGAGCAGCTGAACAACAGCTTCCCCAGCAGCCGGGGCCGGAACA
CCCCCGGCAAGCCCATGCGGGAGGACACCATG
Nterm-R2_R16-R17_R24 to end of dystrophin:
(SEQ ID NO: 38)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGTGGCAGCGGAA
GATCGACGAGACCCTGGAGCGGCTGCAGGAGCTGCAGGAGGCCACCGACGAGCTGGACCTGAAGCTGCGGCAGGCCG
AGGTGATCAAGGGCAGCTGGCAGCCCGTGGGCGACCTGCTGATCGACAGCCTGCAGGACCACCTGGAGAAGGTGAAG
GCCCTGCGGGGCGAGATCGCCCCCCTGAAGGAGAACGTGAGCCACGTGAACGACCTGGCCCGGCAGCTGACCACCCT
GGGCATCCAGCTGAGCCCCTACAACCTGAGCACCCTGGAGGACCTGAACACCCGGTGGAAGCTGCTGCAGGTGGCCG
TGGAGGACCGGGTGCGGCAGCTGCACGAGGCCCACCGGGACTTCGGCCCCGCCAGCCAGCACTTCCTGAGCACCAGC
GTGCAGGGCCCCTGGGAGCGGGCCATCAGCCCCAACAAGGTGCCCTACTACATCAACCACGAGACCCAGACCACCTG
CTGGGACCACCCCAAGATGACCGAGCTGTACCAGAGCCTGGCCGACCTGAACAACGTGCGGTTCAGCGCCTACCGGA
CCGCCATGAAGCTGCGGCGGCTGCAGAAGGCCCTGTGCCTGGACCTGCTGAGCCTGAGCGCCGCCTGCGACGCCCTG
GACCAGCACAACCTGAAGCAGAACGACCAGCCCATGGACATCCTGCAGATCATCAACTGCCTGACCACCATCTACGA
CCGGCTGGAGCAGGAGCACAACAACCTGGTGAACGTGCCCCTGTGCGTGGACATGTGCCTGAACTGGCTGCTGAACG
TGTACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGCCT
GCTGCTGCACGACAGCATCCAGATCCCCCGGCAGCTGGGCGAGGTGGCCAGCTTCGGCGGCAGCAACATCGAGCCCA
GCGTGCGGAGCTGCTTCCAGTTCGCCAACAACAAGCCCGAGATCGAGGCCGCCCTGTTCCTGGACTGGATGCGGCTG
GAGCCCCAGAGCATGGTGTGGCTGCCCGTGCTGCACCGGGTGGCCGCCGCCGAGACCGCCAAGCACCAGGCCAAGTG
CAACATCTGCAAGGAGTGCCCCATCATCGGCTTCCGGTACCGGAGCCTGAAGCACTTCAACTACGACATCTGCCAGA
GCTGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCACCC
CCGGATGGGCTACCTGCCCGTGCAGACCGTGCTGGAGGGCGACAACATGGAGACCCCCGTGACCCTGATCAACTTCT
GGCCCGTGGACAGCGCCCCCGCCAGCAGCCCCCAGCTGAGCCACGACGACACCCACAGCCGGATCGAGCACTACGCC
AGCCGGCTGGCCGAGATGGAGAACAGCAACGGCAGCTACCTGAACGACAGCATCAGCCCCAACGAGAGCATCGACGA
CGAGCACCTGCTGATCCAGCACTACTGCCAGAGCCTGAACCAGGACAGCCCCCTGAGCCAGCCCCGGAGCCCCGCCC
AGATCCTGATCAGCCTGGAGAGCGAGGAGCGGGGCGAGCTGGAGCGGATCCTGGCCGACCTGGAGGAGGAGAACCGG
AACCTGCAGGCCGAGTACGACCGGCTGAAGCAGCAGCACGAGCACAAGGGCCTGAGCCCCCTGCCCAGCCCCCCCGA
GATGATGCCCACCAGCCCCCAGAGCCCCCGGGACGCCGAGCTGATCGCCGAGGCCAAGCTGCTGCGGCAGCACAAGG
GCCGGCTGGAGGCCCGGATGCAGATCCTGGAGGACCACAACAAGCAGCTGGAGAGCCAGCTGCACCGGCTGCGGCAG
CTGCTGGAGCAGCCCCAGGCCGAGGCCAAGGTGAACGGCACCACCGTGAGCAGCCCCAGCACCAGCCTGCAGCGGAG
CGACAGCAGCCAGCCCATGCTGCTGCGGGTGGTGGGCAGCCAGACCAGCGACAGCATGGGCGAGGAGGACCTGCTGA
GCCCCCCCCAGGACACCAGCACCGGCCTGGAGGAGGTGATGGAGCAGCTGAACAACAGCTTCCCCAGCAGCCGGGGC
CGGAACACCCCCGGCAAGCCCATGCGGGAGGACACCATG
Nterm-R2_R16-R17_R24 to end of syntrophin region:
(SEQ ID NO: 39)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCACACCGTGCGGGAGGAGACCATGAT
GGTGATGACCGAGGACATGCCCCTGGAGATCAGCTACGTGCCCAGCACCTACCTGACCGAGATCACCCACGTGAGCC
AGGCCCTGCTGGAGGTGGAGCAGCTGCTGAACGCCCCCGACCTGTGCGCCAAGGACTTCGAGGACCTGTTCAAGCAG
GAGGAGAGCCTGAAGAACATCAAGGACAGCCTGCAGCAGAGCAGCGGCCGGATCGACATCATCCACAGCAAGAAGAC
CGCCGCCCTGCAGAGCGCCACCCCCGTGGAGCGGGTGAAGCTGCAGGAGGCCCTGAGCCAGCTGGACTTCCAGTGGG
AGAAGGTGAACAAGATGTACAAGGACCGGCAGGGCCGGTTCGACCGGAGCGTGGAGAAGTGGCGGCGGTTCCACTAC
GACATCAAGATCTTCAACCAGTGGCTGACCGAGGCCGAGCAGTTCCTGCGGAAGACCCAGATCCCCGAGAACTGGGA
GCACGCCAAGTACAAGTGGTACCTGAAGGAGCTGCAGGACGGCATCGGCCAGCGGCAGACCGTGGTGCGGACCCTGA
ACGCCACCGGCGAGGAGATCATCCAGCAGAGCAGCAAGACCGACGCCAGCATCCTGCAGGAGAAGCTGGGCAGCCTG
AACCTGCGGTGGCAGGAGGTGTGCAAGCAGCTGAGCGACCGGAAGAAGCGGCTGGAGGAGCAGAAGTGGCAGCGGAA
GATCGACGAGACCCTGGAGCGGCTGCAGGAGCTGCAGGAGGCCACCGACGAGCTGGACCTGAAGCTGCGGCAGGCCG
AGGTGATCAAGGGCAGCTGGCAGCCCGTGGGCGACCTGCTGATCGACAGCCTGCAGGACCACCTGGAGAAGGTGAAG
GCCCTGCGGGGCGAGATCGCCCCCCTGAAGGAGAACGTGAGCCACGTGAACGACCTGGCCCGGCAGCTGACCACCCT
GGGCATCCAGCTGAGCCCCTACAACCTGAGCACCCTGGAGGACCTGAACACCCGGTGGAAGCTGCTGCAGGTGGCCG
TGGAGGACCGGGTGCGGCAGCTGCACGAGGCCCACCGGGACTTCGGCCCCGCCAGCCAGCACTTCCTGAGCACCAGC
GTGCAGGGCCCCTGGGAGCGGGCCATCAGCCCCAACAAGGTGCCCTACTACATCAACCACGAGACCCAGACCACCTG
CTGGGACCACCCCAAGATGACCGAGCTGTACCAGAGCCTGGCCGACCTGAACAACGTGCGGTTCAGCGCCTACCGGA
CCGCCATGAAGCTGCGGCGGCTGCAGAAGGCCCTGTGCCTGGACCTGCTGAGCCTGAGCGCCGCCTGCGACGCCCTG
GACCAGCACAACCTGAAGCAGAACGACCAGCCCATGGACATCCTGCAGATCATCAACTGCCTGACCACCATCTACGA
CCGGCTGGAGCAGGAGCACAACAACCTGGTGAACGTGCCCCTGTGCGTGGACATGTGCCTGAACTGGCTGCTGAACG
TGTACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGCCT
GCTGCTGCACGACAGCATCCAGATCCCCCGGCAGCTGGGCGAGGTGGCCAGCTTCGGCGGCAGCAACATCGAGCCCA
GCGTGCGGAGCTGCTTCCAGTTCGCCAACAACAAGCCCGAGATCGAGGCCGCCCTGTTCCTGGACTGGATGCGGCTG
GAGCCCCAGAGCATGGTGTGGCTGCCCGTGCTGCACCGGGTGGCCGCCGCCGAGACCGCCAAGCACCAGGCCAAGTG
CAACATCTGCAAGGAGTGCCCCATCATCGGCTTCCGGTACCGGAGCCTGAAGCACTTCAACTACGACATCTGCCAGA
GCTGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCACCC
CCGGATGGGCTACCTGCCCGTGCAGACCGTGCTGGAGGGCGACAACATGGAGACCCCCGTGACCCTGATCAACTTCT
GGCCCGTGGACAGCGCCCCCGCCAGCAGCCCCCAGCTGAGCCACGACGACACCCACAGCCGGATCGAGCACTACGCC
AGCCGGCTGGCCGAGATGGAGAACAGCAACGGCAGCTACCTGAACGACAGCATCAGCCCCAACGAGAGCATCGACGA
CGAGCACCTGCTGATCCAGCACTACTGCCAGAGCCTGAACCAGGACAGCCCCCTGAGCCAGCCCCGGAGC
Nterm-R2_R17_H3_R22_H4 to end of syntrophin region:
(SEQ ID NO: 117)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCCGAGGACAAGAGCTTCGGCAGCAGCCT
GATGGAGAGCGAGGTGAACCTGGACCGGTACCAGACCGCCCTGGAGGAGGTGCTGAGCTGGCTGCTGAGCGCCGAGG
ACACCCTGCAGGCCCAGGGCGAGATCAGCAACGACGTGGAGGTGGTGAAGGACCAGTTCCACACCCACGAGGGCTAC
ATGATGGACCTGACCGCCCACCAGGGCCGGGTGGGCAACATCCTGCAGCTGGGCAGCAAGCTGATCGGCACCGGCAA
GCTGAGCGAGGACGAGGAGACCGAGGTGCAGGAGCAGATGAACCTGCTGAACAGCCGGTGGGAGTGCCTGCGGGTGG
CCAGCATGGAGAAGCAGAGCAACCTGCACCGGGTGCTGATGGACCTGCAGAACCAGAAGCTGAAGGAGCTGAACGAC
TGGCTGACCAAGACCGAGGAGCGGACCCGGAAGATGGAGGAGGAGCCCCTGGGCCCCGACCTGGAGGACCTGAAGCG
GCAGGTGCAGCAGCACAAGGTGCTGCAGGAGGACCTGGAGCAGGAGCAGGTGCGGGTGAACAGCCTGACCCACATGG
TGGTGGTGGTGGACGAGAGCAGCGGCGACCACGCCACCGCCGCCCTGGAGGAGCAGCTGAAGGTGCTGGGCGACCGG
TGGGCCAACATCTGCCGGTGGACCGAGGACCGGTGGGTGCTGCTGCAGGACCGGTTCGACCGGAGCGTGGAGAAGTG
GCGGCGGTTCCACTACGACATCAAGATCTTCAACCAGTGGCTGACCGAGGCCGAGCAGTTCCTGCGGAAGACCCAGA
TCCCCGAGAACTGGGAGCACGCCAAGTACAAGTGGTACCTGAAGGAGCTGCAGGACGGCATCGGCCAGCGGCAGACC
GTGGTGCGGACCCTGAACGCCACCGGCGAGGAGATCATCCAGCAGAGCAGCAAGACCGACGCCAGCATCCTGCAGGA
GAAGCTGGGCAGCCTGAACCTGCGGTGGCAGGAGGTGTGCAAGCAGCTGAGCGACCGGAAGAAGCGGCTGGAGGAGC
AGAAGCAGCCCGACCTGGCCCCCGGCCTGACCACCATCGGCGCCAGCCCCACCCAGACCGTGACCCTGGTGACCCAG
CCCGTGGTGACCAAGGAGACCGCCATCAGCAAGCTGGAGATGCCCAGCAGCCTGATGCTGGAGGTGCCCGAGGAGAC
CCACCGGCTGCTGCAGCAGTTCCCCCTGGACCTGGAGAAGTTCCTGGCCTGGCTGACCGAGGCCGAGACCACCGCCA
ACGTGCTGCAGGACGCCACCCGGAAGGAGCGGCTGCTGGAGGACAGCAAGGGCGTGAAGGAGCTGATGAAGCAGTGG
CAGGACCTGCAGGGCGAGATCGAGGCCCACACCGACGTGTACCACAACCTGGACGAGAACAGCCAGAAGATCCTGCG
GAGCCTGGAGGGCAGCGACGACGCCGTGCTGCTGCAGCGGCGGCTGGACAACATGAACTTCAAGTGGAGCGAGCTGC
GGAAGAAGAGCCTGAACATCCGGAGCCACCTGGAGGCCAGCAGCGACCAGTGGAAGCGGCTGGGCCCCGCCAGCCAG
CACTTCCTGAGCACCAGCGTGCAGGGCCCCTGGGAGCGGGCCATCAGCCCCAACAAGGTGCCCTACTACATCAACCA
CGAGACCCAGACCACCTGCTGGGACCACCCCAAGATGACCGAGCTGTACCAGAGCCTGGCCGACCTGAACAACGTGC
GGTTCAGCGCCTACCGGACCGCCATGAAGCTGCGGCGGCTGCAGAAGGCCCTGTGCCTGGACCTGCTGAGCCTGAGC
GCCGCCTGCGACGCCCTGGACCAGCACAACCTGAAGCAGAACGACCAGCCCATGGACATCCTGCAGATCATCAACTG
CCTGACCACCATCTACGACCGGCTGGAGCAGGAGCACAACAACCTGGTGAACGTGCCCCTGTGCGTGGACATGTGCC
TGAACTGGCTGCTGAACGTGTACGACACCGGCCGGACCGGCCGGATCCGGGTGCTGAGCTTCAAGACCGGCATCATC
AGCCTGTGCAAGGCCCACCTGGAGGACAAGTACCGGTACCTGTTCAAGCAGGTGGCCAGCAGCACCGGCTTCTGCGA
CCAGCGGCGGCTGGGCCTGCTGCTGCACGACAGCATCCAGATCCCCCGGCAGCTGGGCGAGGTGGCCAGCTTCGGCG
GCAGCAACATCGAGCCCAGCGTGCGGAGCTGCTTCCAGTTCGCCAACAACAAGCCCGAGATCGAGGCCGCCCTGTTC
CTGGACTGGATGCGGCTGGAGCCCCAGAGCATGGTGTGGCTGCCCGTGCTGCACCGGGTGGCCGCCGCCGAGACCGC
CAAGCACCAGGCCAAGTGCAACATCTGCAAGGAGTGCCCCATCATCGGCTTCCGGTACCGGAGCCTGAAGCACTTCA
ACTACGACATCTGCCAGAGCTGCTTCTTCAGCGGCCGGGTGGCCAAGGGCCACAAGATGCACTACCCCATGGTGGAG
TACTGCACCCCCACCACCAGCGGCGAGGACGTGCGGGACTTCGCCAAGGTGCTGAAGAACAAGTTCCGGACCAAGCG
GTACTTCGCCAAGCACCCCCGGATGGGCTACCTGCCCGTGCAGACCGTGCTGGAGGGCGACAACATGGAGACCCCCG
TGACCCTGATCAACTTCTGGCCCGTGGACAGCGCCCCCGCCAGCAGCCCCCAGCTGAGCCACGACGACACCCACAGC
CGGATCGAGCACTACGCCAGCCGGCTGGCCGAGATGGAGAACAGCAACGGCAGCTACCTGAACGACAGCATCAGCCC
CAACGAGAGCATCGACGACGAGCACCTGCTGATCCAGCACTACTGCCAGAGCCTGAACCAGGACAGCCCCCTGAGCC
AGCCCCGGAGC

A nucleotide sequence encoding a micro-dystrophin protein optimized for skeletal muscle is further understood to include nucleotide sequences that are variants of any one of SEQ ID Nos. 22-39, 99, and 116-118. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID Nos. 22-39, 99, and 116-118.

In some embodiments, a micro-dystrophin protein optimized for skeletal muscle is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 99, and 116-118.

In some embodiments, a micro-dystrophin protein optimized for skeletal muscle is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 99, and 116-118.

In some embodiments, a micro-dystrophin protein optimized for skeletal muscle is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 99, and 116-118.

In some embodiments, it is desirable to overexpress micro-dystrophin proteins in skeletal muscle. Thus, in some embodiments, a nucleotide sequence encoding a micro-dystrophin protein optimized for skeletal muscle may be codon optimized for higher expression. In some embodiments, one or more codons (e.g., 1, 2, 3, 4, 5, 6, 7, 8, 9, 10, 11, 12, 13, 14, 15, 16, 17, 18, 19, 20, or more) are substituted.

The “percent identity” of two amino acid sequences or nucleic acid sequences may be determined by any method known in the art.

In some embodiments, the percent identity of two nucleic acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST nucleotide searches can be performed with the NBLAST program, score=100, wordlength-12, to obtain guide sequences homologous to a target nucleic acid. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

### Recombinant Nucleic Acids

The disclosure provides recombinant nucleic acids comprising a nucleotide sequence encoding a micro-dystrophin protein of the disclosure. A recombinant nucleic acid is a molecule that is constructed by joining nucleic acids (e.g., isolated nucleic acids, synthetic nucleic acids or a combination thereof) from multiple sources.

A recombinant nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Recombinant nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

Expression of the micro-dystrophin protein may be controlled using one or more regulatory sequences such as enhancers and promoters, operably linked to the nucleotide sequences encoding the micro-dystrophin protein.

A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence.

Promoters that may be used in accordance with the present disclosure may comprise any suitable promoter that can drive the expression of the nucleotide sequences encoding the micro-dystrophin proteins.

In some embodiments, a promoter is naturally associated with dystrophin, and may be obtained by isolating the 5′ non-coding sequence upstream of the coding segment and/or exon of dystrophin. Such a promoter may be referred to as an endogenous promoter or a native promoter.

In some embodiments, the promoter is a chimeric promoter comprising sequence elements from two or more different promoters.

In some embodiments, the promoter is a constitutively active promoter. Constitutive promoters include any constitutive promoter described herein or known to one of ordinary skill in the art. Non-limiting examples of constitutive promoters include the immediate early cytomegalovirus (CMV) promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as mammalian gene promoters such as, but not limited to, the elongation Factor-la (EF-1a) the actin promoter, the myosin promoter, the hemoglobin promoter, and the creatine kinase promoter.

Inducible promoters are also contemplated herein. An “inducible promoter” refers to a promoter that is characterized by regulating (e.g., initiating or activating) transcriptional activity when in the presence of, influenced by or contacted by an inducer signal.

In some embodiments, the promoter is a tissue-specific promoter. A “tissue-specific promoter”, as used herein, refers to promoters that preferentially or selectively function in a specific type of tissue. In some embodiments, a tissue-specific promoter is not able to drive the expression of the genes in other types of tissues. In some embodiments, a promoter that may be used in accordance with the present disclosure is a skeletal muscle-specific promoter. In some embodiments, the skeletal muscle-specific promoter is the CK6 promoter, CK8 promoter, or skeletal α-actin promoter. In some embodiments, the skeletal muscle-specific promoter comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a promoter that may be used in accordance with the present disclosure is a cardiac-specific promoter. In some embodiments, the cardiac-specific promoter is the cardiac troponin C promoter, the cardiac troponin I promoter, or the cardiac troponin T (cTnT) promoter. In some embodiments, the cardiac-specific promoter is the cTnT promoter.

In some embodiments, the engineered nucleic acids of the present disclosure further comprise one or more enhancer elements. In some embodiments, an enhancer element is a skeletal muscle alpha-actin enhancer. In some embodiments, the promoter is a skeletal muscle-specific promoter with skeletal muscle alpha-actin enhancer elements.

In some embodiments, the recombinant nucleic acids of the present disclosure further comprise additional regulatory sequences, including, without limitation, a 3′ untranslated region (3′UTR), and/or a poly-adenylation (polyA) signal sequence.

In some embodiments, a nucleotide sequence encoding a micro-dystrophin protein optimized for cardiac muscle is operably linked to a cardiac-specific promoter. In some embodiments, a nucleotide sequence encoding a micro-dystrophin protein optimized for skeletal muscle is operably linked to a skeletal muscle-specific promoter. In some embodiments, a nucleotide sequence encoding a micro-dystrophin protein optimized for cardiac muscle is operably linked to a weaker promoter. In some embodiments, the expression of a micro-dystrophin protein optimized for cardiac muscle is 5%, 10%, 155, 20%, 25%, 30%, 35%, 40%, 45%, or 50% lower than the expression of a micro-dystrophin protein optimized for skeletal muscle.

### Skeletal Muscle-Specific Promoter

In one aspect, the present disclosure provides minimal skeletal muscle-specific promoters based on the skeletal alpha-actin promoter. In some embodiments, a skeletal muscle-specific promoter of the disclosure does not comprise any element that mediates a response in cardiac muscle.

In some embodiments, a skeletal muscle-specific promoter of the disclosure is less than about 600 bp in length. In some embodiments, the skeletal muscle-specific promoter is less than about 550 bp in length. In some embodiments, the skeletal muscle-specific promoter is less than about 500 bp in length. In some embodiments, the skeletal muscle-specific promoter is less than about 400 bp in length.

In some embodiments, a skeletal muscle-specific promoter of the disclosure comprises one or more (e.g., 1, 2, 3, or more) copies of the skeletal muscle alpha-actin enhancer. In some embodiments, a skeletal muscle-specific promoter of the disclosure comprises one or more (e.g., 1, 2, 3, or more) copies of an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 8-106 of SEQ ID NO: 76. In some embodiments, the skeletal alpha-actin enhancer comprises nucleotides 8-106 of SEQ ID NO: 76. In some embodiments, the skeletal alpha-actin enhancer consists essentially of nucleotides 8-106 of SEQ ID NO: 76. In some embodiments, the skeletal alpha-actin enhancer consists of nucleotides 8-106 of SEQ ID NO: 76. In some embodiments, a skeletal muscle-specific promoter of the disclosure comprises one or more (e.g., 1, 2, 3, or more) copies of an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 76. In some embodiments, the skeletal alpha-actin enhancer comprises SEQ ID NO: 76. In some embodiments, the skeletal alpha-actin enhancer consists essentially of SEQ ID NO: 76. In some embodiments, the skeletal alpha-actin enhancer consists of SEQ ID NO: 76.

In some embodiments, a skeletal muscle-specific promoter of the disclosure comprises two copies of the skeletal muscle alpha-actin enhancer. In some embodiments, a skeletal muscle-specific promoter of the disclosure comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 8-205 of SEQ ID NO: 79. In some embodiments, the skeletal alpha-actin enhancer comprises nucleotides 8-205 of SEQ ID NO: 79. In some embodiments, the skeletal alpha-actin enhancer consists essentially of nucleotides 8-205 of SEQ ID NO: 79. In some embodiments, the skeletal alpha-actin enhancer consists of nucleotides 8-205 of SEQ ID NO: 79. In some embodiments, a skeletal muscle-specific promoter of the disclosure comprises an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79. In some embodiments, a skeletal muscle-specific promoter of the disclosure comprises an enhancer that consists essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79. In some embodiments, a skeletal muscle-specific promoter of the disclosure comprises an enhancer that consists of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79.

In some embodiments, a skeletal muscle-specific promoter of the disclosure comprises a core promoter sequence that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 1-274 of SEQ ID NO: 77. In some embodiments, the core promoter sequence comprises nucleotides 1-274 of SEQ ID NO: 77. In some embodiments, the core promoter sequence consists essentially of nucleotides 1-274 of SEQ ID NO: 77. In some embodiments, the core promoter sequence consists of nucleotides 1-274 of SEQ ID NO: 77. In some embodiments, a skeletal muscle-specific promoter of the disclosure comprises a core promoter sequence plus Kozak consensus sequence that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 77. In some embodiments, the core promoter sequence plus Kozak consensus sequence comprises SEQ ID NO: 77. In some embodiments, the core promoter sequence plus a Kozak consensus sequence consists essentially of SEQ ID NO: 77. In some embodiments, the core promoter sequence plus a Kozak consensus sequence consists of SEQ ID NO: 77.

In some embodiments, a skeletal muscle-specific promoter of the disclosure comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure consists essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure consists of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure comprises the nucleotide sequence of SEQ ID NO: 78 or SEQ ID NO: 80. In some embodiments, a skeletal muscle-specific promoter of the disclosure consists essentially of the nucleotide sequence of SEQ ID NO: 78 or SEQ ID NO: 80. In some embodiments, a skeletal muscle-specific promoter of the disclosure consists of the nucleotide sequence of SEQ ID NO: 78 or SEQ ID NO: 80.

A skeletal muscle-specific promoter of the disclosure may be used to express any gene of interest in skeletal muscle. Thus, a skeletal muscle-specific promoter of the disclosure may be operably linked to any gene of interest, to express the gene of interest in skeletal muscle. In some embodiments, the gene of interest is a micro-dystrophin gene of the disclosure.

A skeletal muscle-specific promoter of the disclosure may be used may be used for gene therapy for skeletal muscle disorders. In some embodiments, the skeletal muscle disorder is a muscular dystrophy. In some embodiments, the muscular dystrophy is DMD.

### Vectors

In some aspects, the present disclosure provides vectors comprising the recombinant nucleic acids described herein. A vector is any nucleic acid that may be used as a vehicle to deliver exogenous (foreign) genetic material to a cell. A vector, in some embodiments, is a DNA sequence that includes an insert (e.g., a nucleotide sequence encoding a micro-dystrophin protein and a larger sequence that serves as the backbone of the vector. Non-limiting examples of vectors include plasmids, viruses/viral vectors, phagemids, cosmids (comprising a plasmid and Lambda phage cos sequences), and artificial chromosomes, any of which may be used as provided herein. In some embodiments, the vector is a viral vector, such as a viral particle. In some embodiments, the viral vector is an adenovirus, adeno associated virus (AAV), 7-retrovirus, HSV, lentivirus, or Sendai virus vector. In some embodiments, the viral vector is an recombinant AAV (rAAV) vector.

In some embodiments, a nucleic acid of the disclosure is flanked by AAV ITRs for packaging into an rAAV vector. The phrase “rAAV vector” can include a rAAV genome comprising the gene of interest flanked by AAV ITRs, and an rAAV particle comprising an rAAV genome encapsidated with rAAV capsid proteins.

The ITR sequences may be derived from any AAV serotype (e.g., 1, 2, 3, 4, 5, 6, 7, 8, 9, or 10) or may be derived from more than one serotype. In some embodiments, the ITR sequences are the same serotype as the capsid (e.g., AAV8 ITR sequences and AAV8 capsid, etc.). In some embodiments, the ITR sequences are of a different serotype from the capsid.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. YoungJr., Toni Cathomen and Richard Jude Samulski; U.S. Pat. Nos. 5,139,941 and 5,962,313, all of which are incorporated herein by reference).

Further provided herein are rAAV viral particles or rAAV preparations containing such particles. The rAAV particles comprise a viral capsid and an rAAV genome comprising the gene of interest flanked by AAV ITRs, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV genome comprising the gene of interest flanked by AAV ITRs may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. Non-limiting examples of derivatives and pseudotypes include AAVrh.10, AAVrh.74, AAV2/1, AAV2/5, AAV2/6, AAV2/8, AAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). In some embodiments, the capsid of any of the herein disclosed rAAV particles is of the serotype AAV1, AAV2, AAV3, AAV6, AAV7, AAV8, AAVrh.74, AAVrh.10, AAV2/6 or AAV9.

In some embodiments, a nucleotide sequence encoding a micro-dystrophin protein optimized for cardiac muscle and a nucleotide sequence encoding a micro-dystrophin protein optimized for skeletal muscle are packaged in rAAV particles of the same serotype.

In some embodiments, a nucleotide sequence encoding a micro-dystrophin protein optimized for cardiac muscle and a nucleotide sequence encoding a micro-dystrophin protein optimized for skeletal muscle are packaged in rAAV particles of different serotypes.

In some embodiments, a nucleotide sequence encoding a micro-dystrophin protein optimized for cardiac muscle is packaged in an rAAV particle of the serotype AAV1, AAV8, AAV9, AAVrh.74, or AAVrh.10. In some embodiments, a nucleotide sequence encoding a micro-dystrophin protein optimized for cardiac muscle is packaged in an rAAV particle of the serotype AAV9, AAVrh.74, or AAVrh.10. In some embodiments, a nucleotide sequence encoding a micro-dystrophin protein optimized for skeletal muscle is packaged in an rAAV particle of the serotype AAV1, AAV6, AAV7, AAV8, or AAV9. In some embodiments, a nucleotide sequence encoding a micro-dystrophin protein optimized for skeletal muscle is packaged in an rAAV particle of the serotype AAV8.

In some aspects, the methods described herein comprise expressing a micro-dystrophin protein in cardiac or skeletal muscle. The vectors provided herein may be used for gene therapy for treating skeletal muscle disorders in a subject in need thereof. In some embodiments, the vectors provided herein may be used for gene therapy for treating a muscular dystrophy (e.g., DMD) in a subject in need thereof.

### Methods of Use

In some aspects, the present disclosure provides methods of treating DMD.

In one aspect, the present disclosure provides a method of treating DMD in a subject in need thereof, comprising administering to the subject an effective amount of a micro-dystrophin protein of the disclosure.

In another aspect, the present disclosure provides a gene therapy for treating DMD in a subject in need thereof. Accordingly, the present disclosure provides a method of treating DMD in a subject in need thereof, comprising administering to the subject an effective amount of a nucleic acid molecule encoding a micro-dystrophin protein.

In some embodiments, the present disclosure provides a method of treating DMD in a subject in need thereof, comprising administering to the subject an effective amount of a nucleic acid molecule encoding a micro-dystrophin protein optimized for cardiac muscle and an effective amount of a nucleic acid molecule encoding a micro-dystrophin protein optimized for skeletal muscle.

In some embodiments, the present disclosure provides a method of treating DMD in a subject in need thereof, comprising administering to the subject an effective amount of a nucleic acid molecule encoding a micro-dystrophin protein optimized for cardiac muscle to cardiac muscle and an effective amount of a nucleic acid molecule encoding a micro-dystrophin protein optimized for skeletal muscle to skeletal muscle.

In some embodiments, the present disclosure provides a method of treating DMD in a subject in need thereof, the method comprising delivering to the subject a first gene therapy vector (e.g., a first rAAV particle) comprising a nucleotide sequence encoding a first micro-dystrophin protein to cardiac muscle and a second gene therapy vector (e.g., a second rAAV particle) comprising a nucleotide sequence encoding a second micro-dystrophin to skeletal muscle.

The present disclosure thus contemplates methods of expressing one or more micro-dystrophin proteins in a subject for treating DMD, the method comprising administering to a subject in need thereof an effective amount of one or more nucleic acids of the disclosure.

In some embodiments, additional gene therapy approaches are combined with micro-dystrophin.

The terms “subject,” and “patient,” are used interchangeably herein. In some embodiments, a subject is a mammal, such as a human, a nonhuman primate, a dog, a cat, a horse, a sheep, a poultry, a cow, a pig, a mouse, a rat, a rodent, or a goat. In some embodiments, the subject and mammal is a human.

An “effective amount” of the compositions of the disclosure generally refers to an amount sufficient to elicit the desired biological response, e.g., express the micro-dystrophin protein in a target cell, treat DMD, etc. As will be appreciated by those of ordinary skill in this art, the effective amount of an agent described herein may vary depending on such factors as the condition being treated, the mode of administration, and the age, body composition, and health of the subject. Suitable dosage ranges are readily determinable by one skilled in the art.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

### Compositions

In some aspects, the present disclosure provides compositions comprising the polypeptides, nucleic acids, or vectors disclosed herein. For administration to a subject, the polypeptides, engineered nucleic acids, or vectors disclosed herein may be formulated in a composition. In some embodiments, the composition further comprises additional agents (e.g., for specific delivery, increasing half-life, or other therapeutic agents).

In some embodiments, the composition is a pharmaceutical composition. In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Nucleic acids, in some embodiments, may be formulated in a non-viral delivery vehicle. Non-limiting examples of non-viral delivery vehicles include nanoparticles, such as nanocapsules and nanospheres. See, e.g., Sing, R et al. Exp Mol Pathol. 2009; 86(3):215-223. A nanocapsule is often comprised of a polymeric shell encapsulating an agent. Nanospheres are often comprised of a solid polymeric matrix throughout which the agent is dispersed. In some embodiments, the nanoparticle is a lipid particle, such as a liposome. See, e.g., Puri, A et al. Crit Rev Ther Drug Carrier Syst. 2009; 26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283,1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the engineered nucleic acids disclosed herein may be found, for example, in Remington's Pharmaceutical Sciences, 18th edition, Mack Publishing Co., Easton, Pa (1990) (incorporated herein by reference in its entirety).

### Methods of Administration

Any of the polypeptides, nucleic acids, vectors, or compositions disclosed herein may be administered to a subject.

Suitable routes of administration include, without limitation, intravenous, intranasal, intramuscular, intrathecal, or subcutaneous. In some embodiments, a polypeptide, engineered nucleic acid, vector, or composition of the disclosure is administered intravenously, subcutaneously, intramuscularly intrathecally or intranasally. In some embodiments, a polypeptide, nucleic acid, vector, or composition of the disclosure is administered directly (e.g., by direct injection) to one or more cells (e.g., cardiac or skeletal muscle cells), tissues (e.g., cardiac or skeletal muscle), or organs (e.g., heart). Other routes of administration are contemplated herein. The administration route can be changed depending on a number of factors, including the desired cell, tissue, or organ.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intraarticular, and intramuscular administration and formulation.

In one aspect, the present disclosure relates to methods of achieving localized delivery of a gene therapy vector (e.g., an rAAV vector) to a muscle of a subject. Localized delivery to a muscle can be achieved through different methods including, but not limited to, catheter-based methods, local injections, local injection devices, microneedles or drug-eluting stents or drug-eluting implants. In one aspect, the present disclosure relates to catheter-based methods of delivering gene therapy vectors to a muscle. Examples of catheters include, but are not limited to, guiding catheters, microporous infusion catheters, balloon catheters, porous balloon catheters, microporous balloon catheters, retractable-needle catheters, over-the-needle (OTN) catheters, iontophoretic catheters or butterfly catheters. In some embodiments, the muscle is cardiac muscle. In some embodiments, the muscle is skeletal muscle. Non-limiting examples of skeletal muscles include arm muscles, hand muscles, shoulder muscles, chest muscles, neck muscles, muscles of the larynx, scalp muscles, eye muscles, hip muscles, leg muscles and thigh muscles.

In some embodiments, catheter delivery allows the combined delivery of one or more gene therapy vectors to the cardiac muscle and to one or more skeletal muscle(s). In some embodiments, the gene therapy vector delivered to the cardiac muscle is the same as the gene therapy vector delivered to the skeletal muscle(s). In some embodiments, the gene therapy vector delivered to the cardiac muscle is different from the gene therapy vector delivered to the skeletal muscle(s). In some embodiments, the catheter has multiple lumens for the delivery of the different gene therapy vectors. In some embodiments, the catheter has a single lumen. In some embodiments, the different gene therapy vectors are sequentially introduced as the catheter is moved to different arteries. In some embodiments, the combined delivery to cardiac and skeletal muscles comprises advancing a catheter to heart, delivering a gene therapy vector into the left and right coronary arteries, retracting the catheter to the aortic arch, and delivering a gene therapy vector to arteries that irrigate the skeletal muscles (e.g., the subclavian and/or carotid arteries). In some embodiments, the method further comprises retracting the catheter into the descending aorta and delivering the gene therapy to skeletal muscle via descending aortic branches.

In some embodiments, the present disclosure relates to the delivery of a gene therapy vector to an artery via a catheter. Non-limiting examples of arteries include a femoral artery, a subclavian artery, a carotid artery, an axillary artery, a brachial artery, a radial artery, an ulnar artery, an iliac artery, a popliteal artery, a tibial artery, a dorsalis pedis artery and an aorta. In some embodiments, the gene therapy vector is delivered to a subclavian and/or a carotid artery.

In some embodiments, the catheter is first introduced into the femoral artery and advanced to the subclavian and/or carotid arteries. In some embodiments, the catheter is then retracted into the descending aorta to deliver the gene therapy vector to skeletal muscle via descending aortic branches.

In some embodiments, the methods described herein relate to the delivery of a first rAAV to cardiac muscle and a second rAAV to a skeletal muscle, comprising: (i) introducing a catheter into the femoral artery and advancing to the heart; (ii) delivering the first gene therapy vector into the left and right coronary arteries; (iii) retracting the catheter to the aortic arch; and (iv) delivering the second gene therapy vector to the subclavian and/or carotid arteries. In some embodiments, the first and second rAAVs have different capsid serotypes. In some embodiments, the first and second rAAVs have the same capsid serotype. In some embodiments, the first and second rAAVs carry the same therapeutic gene. In some embodiments, the first and second rAAVs carry different therapeutic genes. In some embodiments, a first rAAV carries a micro-dystrophin gene optimized for cardiac muscle and the second rAAV carries a micro-dystrophin gene optimized for skeletal muscle.

In some embodiments, a vasodilator is administered to the subject prior to, or simultaneously with, the introduction of the catheter into an artery for gene therapy vector delivery. Vasodilators are medications that open (dilate) blood vessels. Non-limiting examples of vasodilators are arterial dilators, venous dilators, mixed dilators, nitroprusside, nitroglycerin, nitric oxide, hydralazine, allicin, nitrates, isosorbide dinitrate, isosorbide mononitrate, erythrityl tetranitrate, pentaerythritol tetranitrate, sodium nitroprusside, alpha-adrenoceptor antagonists (alpha blockers), al-adrenoceptor antagonists (e.g., prazosin, terazosin, doxazosin, trimazosin), phentolamine, phenoxybenzamine, sympatholytics, angiotensin converting enzyme (ACE) inhibitors (e.g., benazepril, captopril, enalapril, fosinopril, lisinopril, moexipril, quinapril, ramipril), angiotensin receptor blockers (ARBs) (e.g., candesartan, eprosartan, irbesartan, losartan, olmesartan, telmisartan, azilsartan, valsartan), alpha-2-agonists (e.g., α-methyldopa, clonidine), alpha agonist of the alpha-2 adrenergic receptor (e.g., guanabenz, guanfacine) beta2-adrenoceptor agonists (β2-agonists), beta-adrenoceptor agonist (isoprenaline), beta-1-adrenergic agonist (e.g., dobutamine), calcium-channel blockers (CCBs) (e.g., amlodipine, felodipine, isradipine, nicardipine, nifedipine, nimodipine, nitrendipine, verapamil, diltiazem), centrally acting sympatholytics, direct acting vasodilators, endothelin receptor antagonists (e.g., bosentan, ambrisentan), ganglionic blockers (e.g. trimethaphan camsylate), nitrodilators, phosphodiesterase inhibitors, potassium-channel openers (e.g., minoxidil), renin inhibitors (e.g., aliskiren), PDE3 inhibitors (e.g., milrinone, inamrinone, amrinone, cilostazol), PDE5 inhibitors (e.g., sildenafil, tadalafil), epinephrine, norepinephrine, or dopamine. In some embodiments, the vasodilator is adenosine. In some embodiments, the vasodilator is a PDE5 inhibitor. In some embodiments, the PDE5 inhibitor is sildenafil or tadalafil. In some embodiments, the PDE5 inhibitor is sildenafil.

In some embodiments, the vasodilator is administered 0, 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150 or 180 minutes prior to the delivery of the gene therapy vector.

In some embodiments, administration of the vasodilator is through intravascular injection. In some embodiments, administration of the vasodilator is through intramuscular injection. In some embodiments, administration of the vasodilator is through ingestion. In some embodiments, administration of the vasodilator is through topical application. s In some embodiments, the methods of the present disclosure further comprise inflating a blood pressure cuff on each limb of the subject at the time of delivery of the gene therapy vector.

In some embodiments, the nucleic acids of the disclosure are delivered via an AAV vector. In some embodiments, the number of AAV particles administered to a subject may be on the order ranging from about 10⁹ to about 10¹⁶ particles, or any values in between, such as for example, about 10⁹, 10¹⁰, 10¹¹, 10¹², 10¹³, 10¹⁴, 10¹⁵, or 10¹⁶ particles. In some embodiments, the number of AAV particles administered to a subject may be on the order ranging from about 10⁹ to about 10¹⁶ vector genomes (vgs), or any values in between, such as for example, about 10⁹, 10¹⁰, 10¹¹, 10¹², 10¹³, 10¹⁴, 10¹⁵, or 10¹⁶ vgs. The AAV particles can be administered as a single dose, or divided into two or more administrations as may be required to achieve therapy. In some embodiments, the two or more administrations are within 24 hours of each other.

### EXAMPLES

### Example 1. Micro-Dystrophin Constructs that are Optimized for Either Skeletal Muscle or Cardiac Muscle

Based on studies in mice with micro-dystrophin constructs that are currently in clinical trials, two discoveries were made. First, it was found that the affinity of the micro-dystrophin for its membrane complex is modulated by the inclusion of the first three spectrin-like repeats (FIG. 1). The inclusion of only R1 leads to relatively weak binding, while the presence of R1+R2 or R1+R2+R3 leads to a much higher apparent affinity for the complex.

Secondly, it was discovered that overexpression of a micro-dystrophin containing either R1+R2 or R1+R2+R3 leads to outcompeting utrophin at the cardiac membrane, while not affecting utrophin in skeletal muscle. This displacement of utrophin in the heart leads to a much more rapid onset of cardiomyopathy and heart failure than if no dystrophin is present. On the other hand, a R1 only containing micro-dystrophin does not outcompete utrophin for its membrane binding in the heart to the same extent. Accordingly, a micro-dystrophin that will be beneficial to the heart, can be designed based on including only R1 of the first three repeats.

In the case of skeletal muscle, it was found that the greater the affinity for the complex, the better, since utrophin localization is unaffected. Thus, for skeletal muscle, the ideal constructs contain either R1+R2 or R1+R2+R3. However, in some embodiments, in order to accommodate the full C-terminus, it may be necessary to exclude R2 and only include R1.

Lastly, the loss of regulation of ion channels, including the TRPC1,3,6 channels, leads to residual pathology in skeletal and cardiac muscles that the current micro-dystrophins do not address. Accordingly, in some embodiments, regions in the C-terminus of dystrophin, including the syntrophin binding sites and/or the coiled coil regions that interact with dystrobrevin (FIG. 1), are included in micro-dystrophin.

The following exemplary micro-dystrophin constructs were designed for AAV delivery in view of the above findings. In some embodiments, the micro-dystrophin constructs designed for skeletal muscle are used with a promoter that expresses only in skeletal muscle and not in the heart. In some embodiments, cardiac-specific expression is achieved with the truncated cTnT promoter. In some embodiments, a smaller cTnT promoter is generated to allow use of the largest cardiac micro-dystrophins described below. FIGS. 2A and 2B shows the order of Repeats (R) and hinges (H), as well as the cysteine-rich portion of the C-terminus (CR), which is commonly included in micro-dystrophins and referred to as the truncated C-terminus, which is then followed by the rest of the C-terminus (CT).

TABLE 1
Cardiac micro-dystrophins

| | Size |
|---|---|
| | (amino |
| | acid |
| Elements included in construct | number) |
| Nterm-R1_R17-R19_R24 to trCterm | 1263/1260 |
| Nterm-R1_R17-R19_H4 to 1^{st} coiled coil and proline | 1286/1283 |
| rich region that follows | |
| Nterm-R1_R17-R19_H4 to end of syntrophin region | 1223/1220 |
| Nterm-R1_R17-R19_R24 to 1^{st} coiled coil and proline | 1407/1404 |
| rich region that follows | |
| Nterm-R1_R17-R19_H4 to end of dystrophin | 1418/1415 |
| Nterm-R1_R17-R19_R24 to end of dystrophin | 1539/1536 |
| Nterm-R1_R17_H3_R21-R22_H4 to end of syntrophin | 1287 |
| region | |
| Nterm-R1_R17_R22_H4 to 1^{st} coiled coil | 1198 |
| Nterm-R1_R16-R17_R21-R22_H4 to end of syntrophin | 1363 |
| region | |

TABLE 2
Skeletal Muscle micro-dystrophins

| | Size |
|---|---|
| | (amino |
| | acid |
| Elements included in construct | number) |
| Nterm-R2_R16-R17_R24 to trCterm | 1277 |
| Nterm-R2_R16-R17_H4 to end of syntrophin region | 1237 |
| Nterm-R2_R16-R17_H4 to 1^{st} coiled coil and proline | 1300 |
| rich region that follows | |
| Nterm-R2_R16-R17_R24 to 1^{st} coiled coil and proline | 1421 |
| rich region that follows | |
| Nterm-R2_R23, R24 to end of dystrophin | 1431 |
| Nterm-R2_R16-R17_H4 to end of dystrophin | 1432 |
| Nterm-R1_R16-R17_R24 to end of dystrophin | 1448 |
| Nterm-R2_R16-R17_R24 to end of dystrophin | 1553 |
| Nterm-R2_R16-R17_R24 to end of syntrophin region | 1358 |
| Nterm-R2_R17_H3_R22_H4 to end of syntrophin region | 1287 |

“-” is used to show the end points of domains that are continuously linked while “_” is used to indicate a deletion between the domains. For example, Nterm-R1_R17-R19_R24 to trCterm indicates that the micro-dystrophin comprises the N-terminus region up to and including R1 linked to R17 up to and including R19 linked to R24 up to and including the truncated C-terminus of dystrophin (i.e., the region between R1 and R17, the region between R19 and R24, and the region after the truncated C-terminus are deleted). The amino acid sequences of modules to build the micro-dystrophin proteins described in this example are provided below. The amino acids of the micro-dystrophin constructs in Tables 1 and 2 are provided as SEQ ID Nos. 1-6 and 13-21 elsewhere in the description.

N-terminus through Hinge 1 (325 amino acids)
(SEQ ID NO: 40)
MLWWEEVEDCYEREDVQKKTFTKWVNAQFSKFGKQHIENLFSDLQDGRRLLDLLEGLTGQKLPKEKGSTRVHALNNV
NKALRVLQNNNVDLVNIGSTDIVDGNHKLTLGLIWNIILHWQVKNVMKNIMAGLQQTNSEKILLSWVRQSTRNYPQV
NVINFTTSWSDGLALNALIHSHRPDLFDWNSVVCQQSATQRLEHAFNIARYQLGIEKLLDPEDVDTTYPDKKSILMY
ITSLFQVLPQQVSIEAIQEVEMLPRPPKVTKEEHFQLHHQMHYSQQITVSLAQGYERTSSPKPRFKSYAYTQAAYVT
TSDPTRSPFPSQHLEAP
Repeat 1 (126 amino acids)
(SEQ ID NO: 41)
EDKSFGSSLMESEVNLDRYQTALEEVLSWLLSAEDTLQAQGEISNDVEVVKDQFHTHEGYMMDLTAHQGRVGNILQL
GSKLIGTGKLSEDEETEVQEQMNLLNSRWECLRVASMEKQSNLHRVLMD
Repeat 2 (105 amino acids)
(SEQ ID NO: 42)
LQNQKLKELNDWLTKTEERTRKMEEEPLGPDLEDLKRQVQQHKVLQEDLEQEQVRVNSLTHMVVVVDESSGDHATAA
LEEQLKVLGDRWANICRWTEDRWVLLQD
Repeat 3 + 4 residues of H2 (118 amino acids)
(SEQ ID NO: 43)
ILLKWQRLTEEQCLFSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQP
Repeat 16 + linker (114 + 9 amino acids = 123 amino acids)
(SEQ ID NO: 44)
HTVREETMMVMTEDMPLEISYVPSTYLTEITHVSQALLEVEQLLNAPDLCAKDFEDLFKQEESLKNIKDSLQQSSGR
IDIIHSKKTAALQSATPVERVKLQEALSQLDFQWEKVNKMYKDROG
Repeat 17 (113 amino acids)
(SEQ ID NO: 45)
RFDRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQK
Repeats 17-19 (327 aa)
(SEQ ID NO: 46)
RFDRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEKLE
QVKLLVEELPLRQGILKQLNETGGPVLVSAPISPEEQDKLENKLKQTNLQWIKVSRALPEKQGEIEAQIKDLGQLEK
KLEDLEEQLNHLLLWLSPIRNQLEIYNQPNQEGPFDVKETEIAVQAKQPDVEEILSKGQHLYKEKPATQPVKRKLED
LSSEWKAVNRLLQELRAKQ
Repeats 17-19 (324 aa)
(SEQ ID NO: 81)
RSVEKWRRFHYDIKIFNQWLTEAEQFLRKTQIPENWEHAKYKWYLKELQDGIGQRQTVVRTLNATGEEII
QQSSKTDASILQEKLGSLNLRWQEVCKQLSDRKKRLEEQKNILSEFQRDLNEFVLWLEEADNIASIPLEP
GKEQQLKEKLEQVKLLVEELPLRQGILKQLNETGGPVLVSAPISPEEQDKLENKLKQTNLQWIKVSRALP
EKQGEIEAQIKDLGQLEKKLEDLEEQLNHLLLWLSPIRNQLEIYNQPNQEGPFDVKETEIAVQAKQPDVE
EILSKGQHLYKEKPATQPVKRKLEDLSSEWKAVNRLLQELRAKQ
Hinge 3 (47 aa)
(SEQ ID NO: 119)
QPDLAPGLTTIGASPTQTVTLVTQPVVTKETAISKLEMPSSLMLEVP
Repeat 21 (105 aa)
(SEQ ID NO: 120)
KDSTQWLEAKEEAEQVLGQARAKLESWKEGPYTVDAIQKKITETKQLAKDLRQWQTNVDVANDLALKLLRDYSADDT
RKVHMITENINASWRSIHKRVSEREAAL
Repeat 22 + linker (126 aa)
(SEQ ID NO: 121)
EETHRLLQQFPLDLEKFLAWLTEAETTANVLQDATRKERLLEDSKGVKELMKQWQDLQGEIEAHTDVYHNLDENSQK
ILRSLEGSDDAVLLORRLDNMNFKWSELRKKSLNIRSHLEASSDQWKRL
Repeat 23 (114 aa)
(SEQ ID NO: 47)
HLSLQELLVWLQLKDDELSRQAPIGGDFPAVQKQNDVHRAFKRELKTKEPVIMSTLETVRIFLTEQPLEGLEKLYQE
PRELPPEERAQNVTRLLRKQAEEVNTEWEKLNLHSAD
Repeat 23 (123 amino acids)
(SEQ ID NO: 82)
ASSDQWKRLHLSLQELLVWLQLKDDELSRQAPIGGDFPAVQKQNDVHRAFKRELKTKEPVIMSTLETVRIFLTEQPL
EGLEKLYQEPRELPPEERAQNVTRLLRKQAEEVNTEWEKLNLHSAD
Repeat 24 (121 amino acids)
(SEQ ID NO: 48)
WQRKIDETLERLQELQEATDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDF
Hinge 4 + truncated C terminus (364 amino acids)
(SEQ ID NO: 49)
GPASQHELSTSVQGPWERAISPNKVPYYINHETQTTCWDHPKMTELYQSLADLNNVRESAYRTAMKLRRLQKALCLD
LLSLSAACDALDQHNLKQNDQPMDILQIINCLTTIYDRLEQEHNNLVNVPLCVDMCLNWLLNVYDTGRIGRIRVLSF
KTGIISLCKAHLEDKYRYLFKQVASSTGFCDQRRLGLLLHDSIQIPRQLGEVASEGGSNIEPSVRSCFQFANNKPEI
EAALFLDWMRLEPQSMVWLPVLHRVAAAETAKHQAKCNICKECPIIGFRYRSLKHFNYDICQSCFFSGRVAKGHKMH
YPMVEYCTPTTSGEDVRDFAKVLKNKFRTKRYFAKHPRMGYLPVQTVLEGDNMETP
Syntrophin binding sites (81 amino acids)
(SEQ ID NO: 50)
VTLINFWPVDSAPASSPQLSHDDTHSRIEHYASRLAEMENSNGSYLNDSISPNESIDDEHLLIQHYCQSLNQDSPLS
QPRS
Coiled coil region (195 amino acids)
1st coiled coil adds 47 aa; including proline rich region adds 16 more aa
(total of 63 aa)
(SEQ ID NO: 51)
PAQILISLESEERGELERILADLEEENRNLQAEYDRLKQQHEHKGLSPLPSPPEMMPTSPQSP
2nd coiled coil region to end of dystrophin; including 2nd coiled coil adds
another 61 aa and region after adds 71 more aa (total of 132 aa)
(SEQ ID NO: 52)
RDAELIAEAKLLRQHKGRLEARMQILEDHNKQLESQLHRLRQLLEQPQAEAKVNGTTVSSPSTSLQRSDSSQPMLLR
VVGSQTSDSMGEEDLLSPPQDTSTGLEEVMEQLNNSFPSSRGRNTPGKPMREDTM

The nucleotide sequences of modules to build the micro-dystrophin genes described in this example are provided below. The nucleotide sequences of the micro-dystrophin constructs in Tables 1 and 2 are provided as SEQ ID Nos. 7-12, 22-39, 92-97, 109-114, and 116-118 elsewhere in the description.

N-terminus through Hinge 1
Human sequence without codon alterations:
(SEQ ID NO: 53)
ATGCTTTGGTGGGAAGAAGTAGAGGACTGTTATGAAAGAGAAGATGTTCAAAAGAAAACATTCACAAAATGGGTAAA
TGCACAATTTTCTAAGTTTGGGAAGCAGCATATTGAGAACCTCTTCAGTGACCTACAGGATGGGAGGCGCCTCCTAG
ACCTCCTCGAAGGCCTGACAGGGCAAAAACTGCCAAAAGAAAAAGGATCCACAAGAGTTCATGCCCTGAACAATGTC
AACAAGGCACTGCGGGTTTTGCAGAACAATAATGTTGATTTAGTGAATATTGGAAGTACTGACATCGTAGATGGAAA
TCATAAACTGACTCTTGGTTTGATTTGGAATATAATCCTCCACTGGCAGGTCAAAAATGTAATGAAAAATATCATGG
CTGGATTGCAACAAACCAACAGTGAAAAGATTCTCCTGAGCTGGGTCCGACAATCAACTCGTAATTATCCACAGGTT
AATGTAATCAACTTCACCACCAGCTGGTCTGATGGCCTGGCTTTGAATGCTCTCATCCATAGTCATAGGCCAGACCT
ATTTGACTGGAATAGTGTGGTTTGCCAGCAGTCAGCCACACAACGACTGGAACATGCATTCAACATCGCCAGATATC
AATTAGGCATAGAGAAACTACTCGATCCTGAAGATGTTGATACCACCTATCCAGATAAGAAGTCCATCTTAATGTAC
ATCACATCACTCTTCCAAGTTTTGCCTCAACAAGTGAGCATTGAAGCCATCCAGGAAGTGGAAATGTTGCCAAGGCC
ACCTAAAGTGACTAAAGAAGAACATTTTCAGTTACATCATCAAATGCACTATTCTCAACAGATCACGGTCAGTCTAG
CACAGGGATATGAGAGAACTTCTTCCCCTAAGCCTCGATTCAAGAGCTATGCCTACACACAGGCTGCTTATGTCACC
ACCTCTGACCCTACACGGAGCCCATTTCCTTCACAGCATTTGGAAGCTCCT
Human sequence with codon optimization:
(SEQ ID NO: 54)
ATGCTGTGGTGGGAGGAGGTGGAGGACTGCTACGAGCGGGAGGACGTGCAGAAGAAGACCTTCACCAAGTGGGTGAA
CGCCCAGTTCAGCAAGTTCGGCAAGCAGCACATCGAGAACCTGTTCAGCGACCTGCAGGACGGCCGGCGGCTGCTGG
ACCTGCTGGAGGGCCTGACCGGCCAGAAGCTGCCCAAGGAGAAGGGCAGCACCCGGGTGCACGCCCTGAACAACGTG
AACAAGGCCCTGCGGGTGCTGCAGAACAACAACGTGGACCTGGTGAACATCGGCAGCACCGACATCGTGGACGGCAA
CCACAAGCTGACCCTGGGCCTGATCTGGAACATCATCCTGCACTGGCAGGTGAAGAACGTGATGAAGAACATCATGG
CCGGCCTGCAGCAGACCAACAGCGAGAAGATCCTGCTGAGCTGGGTGCGGCAGAGCACCCGGAACTACCCCCAGGTG
AACGTGATCAACTTCACCACCAGCTGGAGCGACGGCCTGGCCCTGAACGCCCTGATCCACAGCCACCGGCCCGACCT
GTTCGACTGGAACAGCGTGGTGTGCCAGCAGAGCGCCACCCAGCGGCTGGAGCACGCCTTCAACATCGCCCGGTACC
AGCTGGGCATCGAGAAGCTGCTGGACCCCGAGGACGTGGACACCACCTACCCCGACAAGAAGAGCATCCTGATGTAC
ATCACCAGCCTGTTCCAGGTGCTGCCCCAGCAGGTGAGCATCGAGGCCATCCAGGAGGTGGAGATGCTGCCCCGGCC
CCCCAAGGTGACCAAGGAGGAGCACTTCCAGCTGCACCACCAGATGCACTACAGCCAGCAGATCACCGTGAGCCTGG
CCCAGGGCTACGAGCGGACCAGCAGCCCCAAGCCCCGGTTCAAGAGCTACGCCTACACCCAGGCCGCCTACGTGACC
ACCAGCGACCCCACCCGGAGCCCCTTCCCCAGCCAGCACCTGGAGGCCCCC
Repeat 1
Human sequence without codon alterations:
(SEQ ID NO: 55)
GAAGACAAGTCATTTGGCAGTTCATTGATGGAGAGTGAAGTAAACCTGGACCGTTATCAAACAGCTTTAGAAGAAGT
ATTATCGTGGCTTCTTTCTGCTGAGGACACATTGCAAGCACAAGGAGAGATTTCTAATGATGTGGAAGTGGTGAAAG
ACCAGTTTCATACTCATGAGGGGTACATGATGGATTTGACAGCCCATCAGGGCCGGGTTGGTAATATTCTACAATTG
GGAAGTAAGCTGATTGGAACAGGAAAATTATCAGAAGATGAAGAAACTGAAGTACAAGAGCAGATGAATCTCCTAAA
TTCAAGATGGGAATGCCTCAGGGTAGCTAGCATGGAAAAACAAAGCAATTTACATAGAGTTTTAATGGAT
Human sequence with codon optimization:
(SEQ ID NO: 56)
GAGGACAAGAGCTTCGGCAGCAGCCTGATGGAGAGCGAGGTGAACCTGGACCGGTACCAGACCGCCCTGGAGGAGGT
GCTGAGCTGGCTGCTGAGCGCCGAGGACACCCTGCAGGCCCAGGGCGAGATCAGCAACGACGTGGAGGTGGTGAAGG
ACCAGTTCCACACCCACGAGGGCTACATGATGGACCTGACCGCCCACCAGGGCCGGGTGGGCAACATCCTGCAGCTG
GGCAGCAAGCTGATCGGCACCGGCAAGCTGAGCGAGGACGAGGAGACCGAGGTGCAGGAGCAGATGAACCTGCTGAA
CAGCCGGTGGGAGTGCCTGCGGGTGGCCAGCATGGAGAAGCAGAGCAACCTGCACCGGGTGCTGATGGAC
Repeat 2
Human sequence without codon alterations:
(SEQ ID NO: 57)
CTCCAGAATCAGAAACTGAAAGAGTTGAATGACTGGCTAACAAAAACAGAAGAAAGAACAAGGAAAATGGAGGAAGA
GCCTCTTGGACCTGATCTTGAAGACCTAAAACGCCAAGTACAACAACATAAGGTGCTTCAAGAAGATCTAGAACAAG
AACAAGTCAGGGTCAATTCTCTCACTCACATGGTGGTGGTAGTTGATGAATCTAGTGGAGATCACGCAACTGCTGCT
TTGGAAGAACAACTTAAGGTATTGGGAGATCGATGGGCAAACATCTGTAGATGGACAGAAGACCGCTGGGTTCTTTT
ACAAGAC
Human sequence with codon optimization:
(SEQ ID NO: 58)
CTGCAGAACCAGAAGCTGAAGGAGCTGAACGACTGGCTGACCAAGACCGAGGAGCGGACCCGGAAGATGGAGGAGGA
GCCCCTGGGCCCCGACCTGGAGGACCTGAAGCGGCAGGTGCAGCAGCACAAGGTGCTGCAGGAGGACCTGGAGCAGG
AGCAGGTGCGGGTGAACAGCCTGACCCACATGGTGGTGGTGGTGGACGAGAGCAGCGGCGACCACGCCACCGCCGCC
CTGGAGGAGCAGCTGAAGGTGCTGGGCGACCGGTGGGCCAACATCTGCCGGTGGACCGAGGACCGGTGGGTGCTGCT
GCAGGAC
Repeat 16 + linker
Human sequence without codon alterations:
(SEQ ID NO: 59)
CACACTGTCCGTGAAGAAACGATGATGGTGATGACTGAAGACATGCCTTTGGAAATTTCTTATGTGCCTTCTACTTA
TTTGACTGAAATCACTCATGTCTCACAAGCCCTATTAGAAGTGGAACAACTTCTCAATGCTCCTGACCTCTGTGCTA
AGGACTTTGAAGATCTCTTTAAGCAAGAGGAGTCTCTGAAGAATATAAAAGATAGTCTACAACAAAGCTCAGGTCGG
ATTGACATTATTCATAGCAAGAAGACAGCAGCATTGCAAAGTGCAACGCCTGTGGAAAGGGTGAAGCTACAGGAAGC
TCTCTCCCAGCTTGATTTCCAATGGGAAAAAGTTAACAAAATGTACAAGGACCGACAAGGG
Human sequence with codon optimization:
(SEQ ID NO: 60)
CACACCGTGCGGGAGGAGACCATGATGGTGATGACCGAGGACATGCCCCTGGAGATCAGCTACGTGCCCAGCACCTA
CCTGACCGAGATCACCCACGTGAGCCAGGCCCTGCTGGAGGTGGAGCAGCTGCTGAACGCCCCCGACCTGTGCGCCA
AGGACTTCGAGGACCTGTTCAAGCAGGAGGAGAGCCTGAAGAACATCAAGGACAGCCTGCAGCAGAGCAGCGGCCGG
ATCGACATCATCCACAGCAAGAAGACCGCCGCCCTGCAGAGCGCCACCCCCGTGGAGCGGGTGAAGCTGCAGGAGGC
CCTGAGCCAGCTGGACTTCCAGTGGGAGAAGGTGAACAAGATGTACAAGGACCGGCAGGGC
Repeat 17
Human sequence without codon alterations:
(SEQ ID NO: 61)
CGATTTGACAGATCTGTTGAGAAATGGCGGCGTTTTCATTATGATATAAAGATATTTAATCAGTGGCTAACAGAAGC
TGAACAGTTTCTCAGAAAGACACAAATTCCTGAGAATTGGGAACATGCTAAATACAAATGGTATCTTAAGGAACTCC
AGGATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTGTC
AGACAGAAAAAAGAGGCTAGAAGAACAAAAG
Human sequence with codon optimization:
(SEQ ID NO: 62)
CGGTTCGACCGGAGCGTGGAGAAGTGGCGGCGGTTCCACTACGACATCAAGATCTTCAACCAGTGGCTGACCGAGGC
CGAGCAGTTCCTGCGGAAGACCCAGATCCCCGAGAACTGGGAGCACGCCAAGTACAAGTGGTACCTGAAGGAGCTGC
AGGACGGCATCGGCCAGCGGCAGACCGTGGTGCGGACCCTGAACGCCACCGGCGAGGAGATCATCCAGCAGAGCAGC
AAGACCGACGCCAGCATCCTGCAGGAGAAGCTGGGCAGCCTGAACCTGCGGTGGCAGGAGGTGTGCAAGCAGCTGAG
CGACCGGAAGAAGCGGCTGGAGGAGCAGAAG
Repeats 17-19 (encoding 327 aa)
Human sequence without codon alterations:
(SEQ ID NO: 63)
CGATTTGACAGATCTGTTGAGAAATGGCGGCGTTTTCATTATGATATAAAGATATTTAATCAGTGGCTAACAGAAGC
TGAACAGTTTCTCAGAAAGACACAAATTCCTGAGAATTGGGAACATGCTAAATACAAATGGTATCTTAAGGAACTCC
AGGATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTGTC
AGACAGAAAAAAGAGGCTAGAAGAACAAAAGAATATCTTGTCAGAATTTCAAAGAGATTTAAATGAATTTGTTTTAT
GGTTGGAGGAAGCAGATAACATTGCTAGTATCCCACTTGAACCTGGAAAAGAGCAGCAACTAAAAGAAAAGCTTGAG
CAAGTCAAGTTACTGGTGGAAGAGTTGCCCCTGCGCCAGGGAATTCTCAAACAATTAAATGAAACTGGAGGACCCGT
GCTTGTAAGTGCTCCCATAAGCCCAGAAGAGCAAGATAAACTTGAAAATAAGCTCAAGCAGACAAATCTCCAGTGGA
TAAAGGTTTCCAGAGCTTTACCTGAGAAACAAGGAGAAATTGAAGCTCAAATAAAAGACCTTGGGCAGCTTGAAAAA
AAGCTTGAAGACCTTGAAGAGCAGTTAAATCATCTGCTGCTGTGGTTATCTCCTATTAGGAATCAGTTGGAAATTTA
TAACCAACCAAACCAAGAAGGACCATTTGACGTTAAGGAAACTGAAATAGCAGTTCAAGCTAAACAACCGGATGTGG
AAGAGATTTTGTCTAAAGGGCAGCATTTGTACAAGGAAAAACCAGCCACTCAGCCAGTGAAGAGGAAGTTAGAAGAT
CTGAGCTCTGAGTGGAAGGCGGTAAACCGTTTACTTCAAGAGCTGAGGGCAAAGCAG
Repeats 17-19 (encoding 324 aa)
Human sequence without codon alterations:
(SEQ ID NO: 83)
AGATCTGTTGAGAAATGGCGGCGTTTTCATTATGATATAAAGATATTTAATCAGTGGCTAACAGAAGCTGAACAGTT
TCTCAGAAAGACACAAATTCCTGAGAATTGGGAACATGCTAAATACAAATGGTATCTTAAGGAACTCCAGGATGGCA
TTGGGCAGCGGCAAACTGTTGTCAGAACATTGAATGCAACTGGGGAAGAAATAATTCAGCAATCCTCAAAAACAGAT
GCCAGTATTCTACAGGAAAAATTGGGAAGCCTGAATCTGCGGTGGCAGGAGGTCTGCAAACAGCTGTCAGACAGAAA
AAAGAGGCTAGAAGAACAAAAGAATATCTTGTCAGAATTTCAAAGAGATTTAAATGAATTTGTTTTATGGTTGGAGG
AAGCAGATAACATTGCTAGTATCCCACTTGAACCTGGAAAAGAGCAGCAACTAAAAGAAAAGCTTGAGCAAGTCAAG
TTACTGGTGGAAGAGTTGCCCCTGCGCCAGGGAATTCTCAAACAATTAAATGAAACTGGAGGACCCGTGCTTGTAAG
TGCTCCCATAAGCCCAGAAGAGCAAGATAAACTTGAAAATAAGCTCAAGCAGACAAATCTCCAGTGGATAAAGGTTT
CCAGAGCTTTACCTGAGAAACAAGGAGAAATTGAAGCTCAAATAAAAGACCTTGGGCAGCTTGAAAAAAAGCTTGAA
GACCTTGAAGAGCAGTTAAATCATCTGCTGCTGTGGTTATCTCCTATTAGGAATCAGTTGGAAATTTATAACCAACC
AAACCAAGAAGGACCATTTGACGTTAAGGAAACTGAAATAGCAGTTCAAGCTAAACAACCGGATGTGGAAGAGATTT
TGTCTAAAGGGCAGCATTTGTACAAGGAAAAACCAGCCACTCAGCCAGTGAAGAGGAAGTTAGAAGATCTGAGCTCT
GAGTGGAAGGCGGTAAACCGTTTACTTCAAGAGCTGAGGGCAAAGCAG
Hinge 3
Human sequence without codon alterations:
(SEQ ID NO: 100)
CAGCCTGACCTAGCTCCTGGACTGACCACTATTGGAGCCTCTCCTACTCAGACTGTTACTCTGGTGACACAACCTGT
GGTTACTAAGGAAACTGCCATCTCCAAACTAGAAATGCCATCTTCCTTGATGTTGGAGGTACCT
Human Sequences with codon optimization:
(SEQ ID NO: 101)
CAGCCCGACCTGGCCCCCGGCCTGACCACCATCGGCGCCAGCCCCACCCAGACCGTGACCCTGGTGACCCAGCCCGT
GGTGACCAAGGAGACCGCCATCAGCAAGCTGGAGATGCCCAGCAGCCTGATGCTGGAGGTGCCC
Repeat 21
Human sequence without codon alterations:
(SEQ ID NO: 102)
AAGGATTCAACACAATGGCTGGAAGCTAAGGAAGAAGCTGAGCAGGTCTTAGGACAGGCCAGAGCCAAGCTTGAGTC
ATGGAAGGAGGGTCCCTATACAGTAGATGCAATCCAAAAGAAAATCACAGAAACCAAGCAGTTGGCCAAAGACCTCC
GCCAGTGGCAGACAAATGTAGATGTGGCAAATGACTTGGCCCTGAAACTTCTCCGGGATTATTCTGCAGATGATACC
AGAAAAGTCCACATGATAACAGAGAATATCAATGCCTCTTGGAGAAGCATTCATAAAAGGGTGAGTGAGCGAGAGGC
TGCTTTG
Human Sequences with codon optimization:
(SEQ ID NO: 103)
AAGGACAGCACCCAGTGGCTGGAGGCCAAGGAGGAGGCCGAGCAGGTGCTGGGCCAGGCCCGGGCCAAGCTGGAGAG
CTGGAAGGAGGGCCCCTACACCGTGGACGCCATCCAGAAGAAGATCACCGAGACCAAGCAGCTGGCCAAGGACCTGC
GGCAGTGGCAGACCAACGTGGACGTGGCCAACGACCTGGCCCTGAAGCTGCTGCGGGACTACAGCGCCGACGACACC
CGGAAGGTGCACATGATCACCGAGAACATCAACGCCAGCTGGCGGAGCATCCACAAGCGGGTGAGCGAGCGGGAGGC
CGCCCTG
Repeat 22 + linker
Human sequence without codon alterations:
(SEQ ID NO: 104)
GAAGAAACTCATAGATTACTGCAACAGTTCCCCCTGGACCTGGAAAAGTTTCTTGCCTGGCTTACAGAAGCTGAAAC
AACTGCCAATGTCCTACAGGATGCTACCCGTAAGGAAAGGCTCCTAGAAGACTCCAAGGGAGTAAAAGAGCTGATGA
AACAATGGCAAGACCTCCAAGGTGAAATTGAAGCTCACACAGATGTTTATCACAACCTGGATGAAAACAGCCAAAAA
ATCCTGAGATCCCTGGAAGGTTCCGATGATGCAGTCCTGTTACAAAGACGTTTGGATAACATGAACTTCAAGTGGAG
TGAACTTCGGAAAAAGTCTCTCAACATTAGGTCCCATTTGGAAGCCAGTTCTGACCAGTGGAAGCGTCTG
Human Sequences with codon optimization:
(SEQ ID NO: 105)
GAGGAGACCCACCGGCTGCTGCAGCAGTTCCCCCTGGACCTGGAGAAGTTCCTGGCCTGGCTGACCGAGGCCGAGAC
CACCGCCAACGTGCTGCAGGACGCCACCCGGAAGGAGCGGCTGCTGGAGGACAGCAAGGGCGTGAAGGAGCTGATGA
AGCAGTGGCAGGACCTGCAGGGCGAGATCGAGGCCCACACCGACGTGTACCACAACCTGGACGAGAACAGCCAGAAG
ATCCTGCGGAGCCTGGAGGGCAGCGACGACGCCGTGCTGCTGCAGCGGCGGCTGGACAACATGAACTTCAAGTGGAG
CGAGCTGCGGAAGAAGAGCCTGAACATCCGGAGCCACCTGGAGGCCAGCAGCGACCAGTGGAAGCGGCTG
Repeat 23
Human sequence without codon alterations:
(SEQ ID NO: 64)
CACCTTTCTCTGCAGGAACTTCTGGTGTGGCTACAGCTGAAAGATGATGAATTAAGCCGGCAGGCACCTATTGGAGG
CGACTTTCCAGCAGTTCAGAAGCAGAACGATGTACATAGGGCCTTCAAGAGGGAATTGAAAACTAAAGAACCTGTAA
TCATGAGTACTCTTGAGACTGTACGAATATTTCTGACAGAGCAGCCTTTGGAAGGACTAGAGAAACTCTACCAGGAG
CCCAGAGAGCTGCCTCCTGAGGAGAGAGCCCAGAATGTCACTCGGCTTCTACGAAAGCAGGCTGAGGAGGTCAATAC
TGAGTGGGAAAAATTGAACCTGCACTCCGCTGAC
Human Sequences with codon optimization:
(SEQ ID NO: 65)
CACCTGAGCCTGCAGGAGCTGCTGGTGTGGCTGCAGCTGAAGGACGACGAGCTGAGCCGGCAGGCCCCCATCGGCGG
CGACTTCCCCGCCGTGCAGAAGCAGAACGACGTGCACCGGGCCTTCAAGCGGGAGCTGAAGACCAAGGAGCCCGTGA
TCATGAGCACCCTGGAGACCGTGCGGATCTTCCTGACCGAGCAGCCCCTGGAGGGCCTGGAGAAGCTGTACCAGGAG
CCCCGGGAGCTGCCCCCCGAGGAGCGGGCCCAGAACGTGACCCGGCTGCTGCGGAAGCAGGCCGAGGAGGTGAACAC
CGAGTGGGAGAAGCTGAACCTGCACAGCGCCGAC
Repeat 23 (encoding 123 aa)
Human sequence without codon alterations:
(SEQ ID NO: 84)
GCCAGTTCTGACCAGTGGAAGCGTCTGCACCTTTCTCTGCAGGAACTTCTGGTGTGGCTACAGCTGAAAGATGATGA
ATTAAGCCGGCAGGCACCTATTGGAGGCGACTTTCCAGCAGTTCAGAAGCAGAACGATGTACATAGGGCCTTCAAGA
GGGAATTGAAAACTAAAGAACCTGTAATCATGAGTACTCTTGAGACTGTACGAATATTTCTGACAGAGCAGCCTTTG
GAAGGACTAGAGAAACTCTACCAGGAGCCCAGAGAGCTGCCTCCTGAGGAGAGAGCCCAGAATGTCACTCGGCTTCT
ACGAAAGCAGGCTGAGGAGGTCAATACTGAGTGGGAAAAATTGAACCTGCACTCCGCTGAC
Human sequence with codon optimization:
(SEQ ID NO: 85)
GCCAGCAGCGACCAGTGGAAGCGGCTGCACCTGAGCCTGCAGGAGCTGCTGGTGTGGCTGCAGCTGAAGGACGACGA
GCTGAGCCGGCAGGCCCCCATCGGCGGCGACTTCCCCGCCGTGCAGAAGCAGAACGACGTGCACCGGGCCTTCAAGC
GGGAGCTGAAGACCAAGGAGCCCGTGATCATGAGCACCCTGGAGACCGTGCGGATCTTCCTGACCGAGCAGCCCCTG
GAGGGCCTGGAGAAGCTGTACCAGGAGCCCCGGGAGCTGCCCCCCGAGGAGCGGGCCCAGAACGTGACCCGGCTGCT
GCGGAAGCAGGCCGAGGAGGTGAACACCGAGTGGGAGAAGCTGAACCTGCACAGCGCCGAC
Repeat 24
Human Sequences without codon alterations:
(SEQ ID NO: 66)
TGGCAGAGAAAAATAGATGAGACCCTTGAAAGACTCCGGGAACTTCAAGAGGCCACGGATGAGCTGGACCTCAAGCT
GCGCCAAGCTGAGGTGATCAAGGGATCCTGGCAGCCCGTGGGCGATCTCCTCATTGACTCTCTCCAAGATCACCTCG
AGAAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTTCT
GCAGGTGGCCGTCGAGGACCGAGTCAGGCAGCTGCATGAAGCCCACAGGGACTTT
Human Sequences with codon optimization:
(SEQ ID NO: 67)
TGGCAGCGGAAGATCGACGAGACCCTGGAGCGGCTGCAGGAGCTGCAGGAGGCCACCGACGAGCTGGACCTGAAGCT
GCGGCAGGCCGAGGTGATCAAGGGCAGCTGGCAGCCCGTGGGCGACCTGCTGATCGACAGCCTGCAGGACCACCTGG
AGAAGGTGAAGGCCCTGCGGGGCGAGATCGCCCCCCTGAAGGAGAACGTGAGCCACGTGAACGACCTGGCCCGGCAG
CTGACCACCCTGGGCATCCAGCTGAGCCCCTACAACCTGAGCACCCTGGAGGACCTGAACACCCGGTGGAAGCTGCT
GCAGGTGGCCGTGGAGGACCGGGTGCGGCAGCTGCACGAGGCCCACCGGGACTTC
Hinge 4 + truncated C terminus
Human Sequences without codon alterations:
(SEQ ID NO: 68)
GGTCCAGCATCTCAGCACTTTCTTTCCACGTCTGTCCAGGGTCCCTGGGAGAGAGCCATCTCGCCAAACAAAGTGCC
CTACTATATCAACCACGAGACTCAAACAACTTGCTGGGACCATCCCAAAATGACAGAGCTCTACCAGTCTTTAGCTG
ACCTGAATAATGTCAGATTCTCAGCTTATAGGACTGCCATGAAACTCCGAAGACTGCAGAAGGCCCTTTGCTTGGAT
CTCTTGAGCCTGTCAGCTGCATGTGATGCCTTGGACCAGCACAACCTCAAGCAAAATGACCAGCCCATGGATATCCT
GCAGATTATTAATTGTTTGACCACTATTTATGACCGCCTGGAGCAAGAGCACAACAATTTGGTCAACGTCCCTCTCT
GCGTGGATATGTGTCTGAACTGGCTGCTGAATGTTTATGATACGGGACGAACAGGGAGGATCCGTGTCCTGTCTTTT
AAAACTGGCATCATTTCCCTGTGTAAAGCACATTTGGAAGACAAGTACAGATACCTTTTCAAGCAAGTGGCAAGTTC
AACAGGATTTTGTGACCAGCGCAGGCTGGGCCTCCTTCTGCATGATTCTATCCAAATTCCAAGACAGTTGGGTGAAG
TTGCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTGGC
TGCTGCAGAAACTGCCAAGCATCAGGCCAAATGTAACATCTGCAAAGAGTGTCCAATCATTGGATTCAGGTACAGGA
GTCTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAACAA
ATTTCGAACCAAAAGGTATTTTGCGAAGCATCCCCGAATGGGCTACCTGCCAGTGCAGACTGTCTTAGAGGGGGACA
ACATGGAAACTCCC
Human Sequences with codon optimization:
(SEQ ID NO: 69)
GGCCCCGCCAGCCAGCACTTCCTGAGCACCAGCGTGCAGGGCCCCTGGGAGCGGGCCATCAGCCCCAACAAGGTGCC
CTACTACATCAACCACGAGACCCAGACCACCTGCTGGGACCACCCCAAGATGACCGAGCTGTACCAGAGCCTGGCCG
ACCTGAACAACGTGCGGTTCAGCGCCTACCGGACCGCCATGAAGCTGCGGCGGCTGCAGAAGGCCCTGTGCCTGGAC
CTGCTGAGCCTGAGCGCCGCCTGCGACGCCCTGGACCAGCACAACCTGAAGCAGAACGACCAGCCCATGGACATCCT
GCAGATCATCAACTGCCTGACCACCATCTACGACCGGCTGGAGCAGGAGCACAACAACCTGGTGAACGTGCCCCTGT
GCGTGGACATGTGCCTGAACTGGCTGCTGAACGTGTACGACACCGGCCGGACCGGCCGGATCCGGGTGCTGAGCTTC
AAGACCGGCATCATCAGCCTGTGCAAGGCCCACCTGGAGGACAAGTACCGGTACCTGTTCAAGCAGGTGGCCAGCAG
CACCGGCTTCTGCGACCAGCGGCGGCTGGGCCTGCTGCTGCACGACAGCATCCAGATCCCCCGGCAGCTGGGCGAGG
TGGCCAGCTTCGGCGGCAGCAACATCGAGCCCAGCGTGCGGAGCTGCTTCCAGTTCGCCAACAACAAGCCCGAGATC
GAGGCCGCCCTGTTCCTGGACTGGATGCGGCTGGAGCCCCAGAGCATGGTGTGGCTGCCCGTGCTGCACCGGGTGGC
CGCCGCCGAGACCGCCAAGCACCAGGCCAAGTGCAACATCTGCAAGGAGTGCCCCATCATCGGCTTCCGGTACCGGA
GCCTGAAGCACTTCAACTACGACATCTGCCAGAGCTGCTTCTTCAGCGGCCGGGTGGCCAAGGGCCACAAGATGCAC
TACCCCATGGTGGAGTACTGCACCCCCACCACCAGCGGCGAGGACGTGCGGGACTTCGCCAAGGTGCTGAAGAACAA
GTTCCGGACCAAGCGGTACTTCGCCAAGCACCCCCGGATGGGCTACCTGCCCGTGCAGACCGTGCTGGAGGGCGACA
ACATGGAGACCCCC
Syntrophin binding sites
Human Sequences without codon alterations:
(SEQ ID NO: 70)
GTTACTCTGATCAACTTCTGGCCAGTAGATTCTGCGCCTGCCTCGTCCCCTCAGCTTTCACACGATGATACTCATTC
ACGCATTGAACATTATGCTAGCAGGCTAGCAGAAATGGAAAACAGCAATGGATCTTATCTAAATGATAGCATCTCTC
CTAATGAGAGCATAGATGATGAACATTTGTTAATCCAGCATTACTGCCAAAGTTTGAACCAGGACTCCCCCCTGAGC
CAGCCTCGTAGT
Human Sequences with codon optimization:
(SEQ ID NO: 71)
GTGACCCTGATCAACTTCTGGCCCGTGGACAGCGCCCCCGCCAGCAGCCCCCAGCTGAGCCACGACGACACCCACAG
CCGGATCGAGCACTACGCCAGCCGGCTGGCCGAGATGGAGAACAGCAACGGCAGCTACCTGAACGACAGCATCAGCC
CCAACGAGAGCATCGACGACGAGCACCTGCTGATCCAGCACTACTGCCAGAGCCTGAACCAGGACAGCCCCCTGAGC
CAGCCCCGGAGC
Coiled coil region
1st coiled coil plus proline rich region
Human sequence without codon alterations:
(SEQ ID NO: 72)
CCTGCCCAGATCTTGATTTCCTTAGAGAGTGAGGAAAGAGGGGAGCTAGAGAGAATCCTAGCAGATCTTGAGGAAGA
AAACAGGAATCTGCAAGCAGAATATGACCGTCTAAAGCAGCAGCACGAACATAAAGGCCTGTCCCCACTGCCGTCCC
CTCCTGAAATGATGCCCACCTCTCCCCAGAGTCCC
Human sequence with codon optimization:
(SEQ ID NO: 73)
CCCGCCCAGATCCTGATCAGCCTGGAGAGCGAGGAGCGGGGCGAGCTGGAGCGGATCCTGGCCGACCTGGAGGAGGA
GAACCGGAACCTGCAGGCCGAGTACGACCGGCTGAAGCAGCAGCACGAGCACAAGGGCCTGAGCCCCCTGCCCAGCC
CCCCCGAGATGATGCCCACCAGCCCCCAGAGCCCC
2nd coiled coil region to end of dystrophin
Human sequence without codon alterations:
(SEQ ID NO: 74)
CGGGATGCTGAGCTCATTGCTGAGGCCAAGCTACTGCGTCAACACAAAGGCCGCCTGGAAGCCAGGATGCAAATCCT
GGAAGACCACAATAAACAGCTGGAGTCACAGTTACACAGGCTAAGGCAGCTGCTGGAGCAACCCCAGGCAGAGGCCA
AAGTGAATGGCACAACGGTGTCCTCTCCTTCTACCTCTCTACAGAGGTCCGACAGCAGTCAGCCTATGCTGCTCCGA
GTGGTTGGCAGTCAAACTTCGGACTCCATGGGTGAGGAAGATCTTCTCAGTCCTCCCCAGGACACAAGCACAGGGTT
AGAGGAGGTGATGGAGCAACTCAACAACTCCTTCCCTAGTTCAAGAGGAAGAAATACCCCTGGAAAGCCAATGAGAG
AGGACACAATG
Human sequence with codon optimization:
(SEQ ID NO: 75)
CGGGACGCCGAGCTGATCGCCGAGGCCAAGCTGCTGCGGCAGCACAAGGGCCGGCTGGAGGCCCGGATGCAGATCCT
GGAGGACCACAACAAGCAGCTGGAGAGCCAGCTGCACCGGCTGCGGCAGCTGCTGGAGCAGCCCCAGGCCGAGGCCA
AGGTGAACGGCACCACCGTGAGCAGCCCCAGCACCAGCCTGCAGCGGAGCGACAGCAGCCAGCCCATGCTGCTGCGG
GTGGTGGGCAGCCAGACCAGCGACAGCATGGGCGAGGAGGACCTGCTGAGCCCCCCCCAGGACACCAGCACCGGCCT
GGAGGAGGTGATGGAGCAGCTGAACAACAGCTTCCCCAGCAGCCGGGGCCGGAACACCCCCGGCAAGCCCATGCGGG
AGGACACCATG

### Additional Studies

Four AAV vectors, AAVRH10 CK8.pDys_Nterm-R1_R17_H3_R21-R22_H4 to end of syntrophin region, AAVRH10-CK8. μDys_Nterm-R2_R17_H3_R22_H4 to end of syntrophin region; AAVRH10-CK8. pDys_Nterm-R1_R17_R22_H4 to 1st coiled coil; and AAVRH10-CK8. μDys_Nterm-R1_R16-R17_R21-R22_H4 to end of syntrophin region) were injected (via tail vein) at a dosage of 2×10¹⁴ vector genomes/kg into 5-week-old male D2.mdx mice (dystrophin-deficient mice in a DBA2/J genetic background). In parallel, D2.mdx. mice were also treated with three other micro-dystrophins—PF-06939926 (Pfizer), SRP-9001-10 (Sarepta), and SGT-001 (Solid Bio)—that are currently in clinical trials. A subset of mice were sacrificed at 9 weeks of age to analyze the degree of skeletal muscle protection and rescue (skeletal muscle functional loss of function is apparent by this time). Cardiac functional deficits are not apparent until >9 months of age. Therefore, two groups of mice were analyzed for preservation of cardiac function (cardiac output and normal ventricular diameter) at 12 months of age.

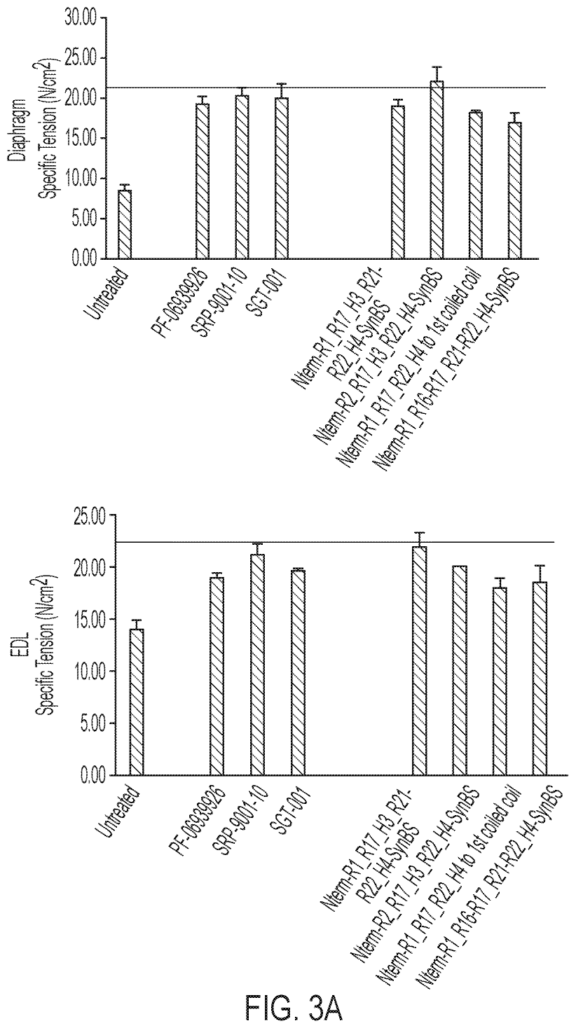

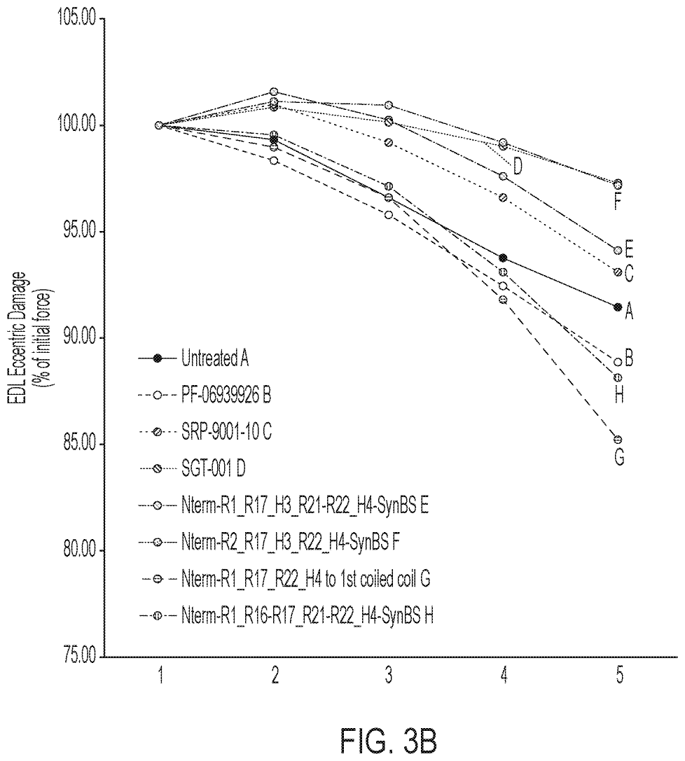

Skeletal muscle analyses revealed that AAVRH10-CK8. μDys_Nterm-R2_R17_H3_R22_H4 to end of syntrophin region improved maintenance of force production in diaphragm and EDL muscles (FIG. 3A). This AAV vector also exhibited enhanced protection of skeletal muscles from contraction-induced injury (see FIG. 3B). AAVRH10-CK8. μDys_Nterm-R1_R17_H3_R21-R22_H4 to end of syntrophin region also exhibited improved muscle function and resistance to injury (FIGS. 3A-3B).

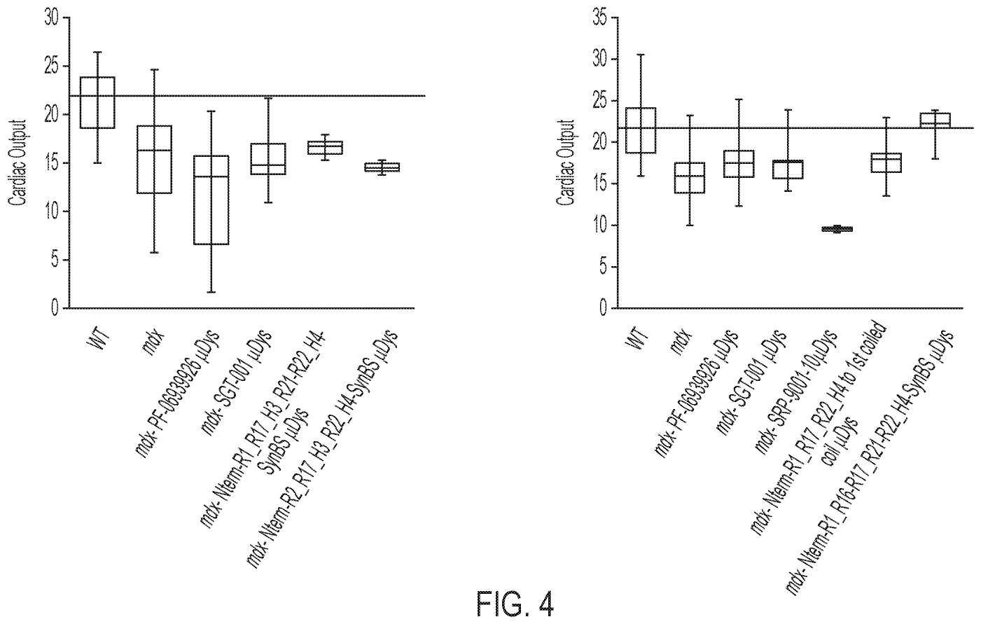

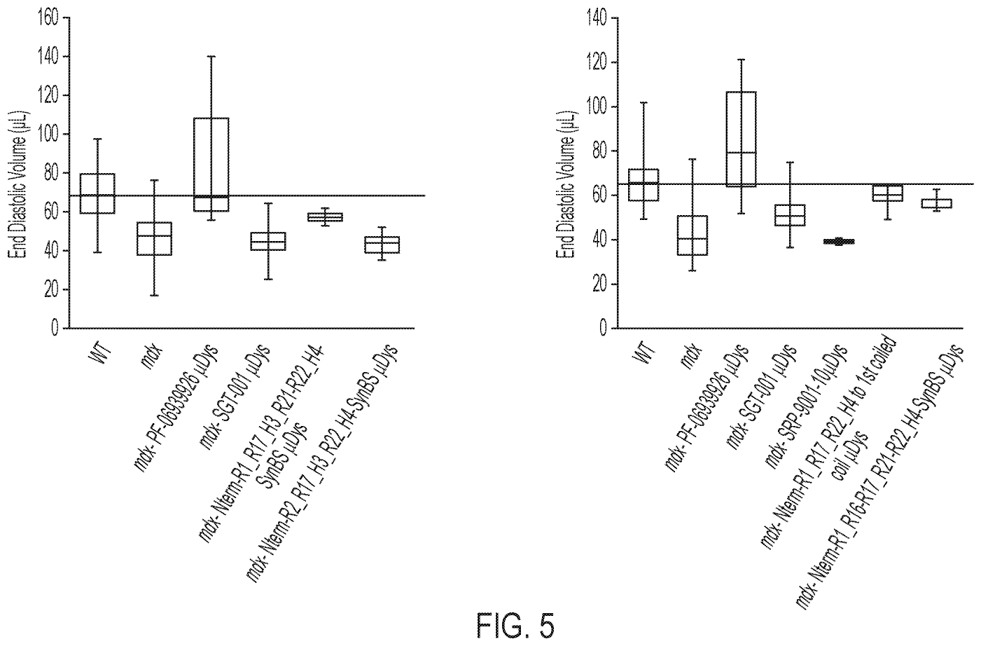

Heart analyses revealed that AAVRH10-CK8. μDys_Nterm-R1_R16-R17_R21-R22_H4 to end of syntrophin region improved cardiac output (see. FIG. 4) and ventricular end diastolic volumes (EDV) (see FIG. 5) of D2.mdx mice. Administration of AAVRH10-CK8. μDys_Nterm-R1_R17_R22_H4 to 1st coiled coil and AAVRH10-CK8. μDys_Nterm-R1_R17_H3_R21-R22_H4 to end of syntrophin region also improved cardiac output (see FIG. 4) and EDV (see FIG. 5) in D2.mdx mice. These results demonstrate that micro-dystrophins of the present disclosure improve skeletal muscle and cardiac function in a manner that is comparable to or superior to the known micro-dystrophins that are currently in clinical trials.

### Example 2. New Skeletal Muscle-Specific Promoter

The finding that the heart and skeletal muscle may need different micro-dystrophin constructs delivered to each muscle type, motivated the need for a small, skeletal muscle-specific promoter. Elements from the skeletal muscle alpha-actin promoter were taken to construct such a promoter. It consists of one or two copies of the 99 bp skeletal muscle alpha-actin enhancer and 274 bases of the core promoter elements, resulting in a 380 bp construct (or 479 bp with 2 enhancers).

Sequences of the promoter are provided below.

### a) 380 bp Construct

Enhancer
(XbaI site for cutting 5′ end)
(SEQ ID NO: 76)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGC
Core Promoter + KOZAK
(SEQ ID NO: 77)
AGGAGGGGCAAACCCGCTAGGGAGACACTCCATATACGGCCCGGCCCGCG
TTACCTGGGACCGGGCCAACCCGCTCCTTCTTTGGTCAACGCAGGGGACC
CGGGCGGGGGCCCAGGCCGCGAACCGGCCGAGGGGGGGGCTCTAGTGCCC
AACACCCAAATATGGCTCGAGAAGGGCAGCGACATTCCTGCGGGGTGGCG
CGGAGGGAATGCCCGCGGGCTATATAAAACCTGAGCAGAGGGACAAGCGG
CCACCGCAGCGGACAGCGCCACC ATGG

ATG+G—initiation codon+G of coding sequence follows and completes the optimal KOZAK and creates a NcoI site (CCATGG) to cut the full promoter. The G after the ATG ia not necessary if other cloning strategies are preferred.

The promoter is 380 bp in length:

(SEQ ID NO: 78)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGCAGGAGGGGCAAACCCGCTAGGGAGACACTCCATATACGGCCCGG
CCCGCGTTACCTGGGACCGGGCCAACCCGCTCCTTCTTTGGTCAACGCAG
GGGACCCGGGCGGGGGCCCAGGCCGCGAACCGGCCGAGGGAGGGGGCTCT
AGTGCCCAACACCCAAATATGGCTCGAGAAGGGCAGCGACATTCCTGCGG
GGTGGCGCGGAGGGAATGCCCGCGGGCTATATAAAACCTGAGCAGAGGGA
CAAGCGGCCACCGCAGCGGACAGCGCCACC

### b) 479 bp Construct (Stronger Promoter)

Back to back Enhancers
(XbaI site for cutting 5′ end)
(SEQ ID NO: 79)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGCAATCTGAAAGGCATAGCCCCATATATCAGTGATATAAATAGAAC
CTGCAGCAGGCTCTGGTAAATGATGACTACAAGGTGGACTGGGAGGCAGC
CCGGC
Core Promoter + KOZAK
(SEQ ID NO: 77)
AGGAGGGGCAAACCCGCTAGGGAGACACTCCATATACGGCCCGGCCCGCG
TTACCTGGGACCGGGCCAACCCGCTCCTTCTTTGGTCAACGCAGGGGACC
CGGGCGGGGGCCCAGGCCGCGAACCGGCCGAGGGAGGGGGCTCTAGTGCC
CAACACCCAAATATGGCTCGAGAAGGGCAGCGACATTCCTGCGGGGTGGC
GCGGAGGGAATGCCCGCGGGCTATATAAAACCTGAGCAGAGGGACAAGCG
GCCACCGCAGCGGACAGCGCCACCATGG

The promoter is 479 bp in length:

(SEQ ID NO: 80)
TCTAGAAAATCTGAAAGGCATAGCCCCATATATCAGTGATATAAATAG
AACCTGCAGCAGGCTCTGGTAAATGATGACTACAAGGTGGACTGGGAG
GCAGCCCGGCAATCTGAAAGGCATAGCCCCATATATCAGTGATATAAA
TAGAACCTGCAGCAGGCTCTGGTAAATGATGACTACAAGGTGGACTGG
GAGGCAGCCCGGCAGGAGGGGCAAACCCGCTAGGGAGACACTCCATAT
ACGGCCCGGCCCGCGTTACCTGGGACCGGGCCAACCCGCTCCTTCTTT
GGTCAACGCAGGGGACCCGGGCGGGGGCCCAGGCCGCGAACCGGCCGA
GGGAGGGGGCTCTAGTGCCCAACACCCAAATATGGCTCGAGAAGGGCA
GCGACATTCCTGCGGGGTGGCGCGGAGGGAATGCCCGCGGGCTATATA
AAACCTGAGCAGAGGGACAAGCGGCCACCGCAGCGGACAGCGCCACC

### Example 3. New Approach for rAAV Delivery to Muscle

If both the heart and skeletal muscle are to be optimally targeted in rAAV gene delivery, then it would be best to deliver the cardiac rAAV and the skeletal muscle rAAV via the specific arterial beds for the muscles, rather than the intravenous delivery as is the common practice. This is accomplished using a drug delivery catheter that is introduced into the femoral artery and first advanced the heart. The cardiac rAAV is first delivered into the left and right coronary arteries. Following the cardiac rAAV delivery, the catheter is retracted to the aortic arch, where skeletal muscle-targeted rAAV is then delivered to the subclavian arteries and, if desired, the carotid arteries. The catheter is then retracted into the descending aorta, if desired, and the rest of the skeletal muscle rAAV is delivered via various descending aortic branches, depending on the desired skeletal muscle distribution.

To further enhance distribution of rAAV to skeletal muscle, a PDE5 inhibitor, such as sildenafil or tadalafil, is given 1 hour prior to rAAV delivery to increase blood flow to resting skeletal muscle. Additionally, inflation of a blood pressure cuff on each limb at a pressure below the systolic pressure and above the diastolyic pressure improves retention of virus in the limbs at the time of viral delivery.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A recombinant nucleic acid comprising a nucleotide sequence encoding a micro-dystrophin protein comprising: an amino-terminal actin-binding domain, hinge domain 1, spectrin-like repeat 1, and spectrin-like repeat 17, wherein the micro-dystrophin protein does not contain spectrin-like repeat 2 and/or spectrin-like repeat 3 of dystrophin, and wherein the micro-dystrophin protein optionally further comprises spectrin-like repeat 18 and/or spectrin-like repeat 19.

2. The recombinant nucleic acid of claim 1, wherein the micro-dystrophin protein further comprises one or more of: spectrin-like repeat 16, hinge domain 3, spectrin-like repeat 21, spectrin-like repeat 22, spectrin-like repeat 24, hinge domain 4, and dystroglycan binding site of dystrophin.

3. The recombinant nucleic acid of claim 1 or 2, wherein the micro-dystrophin protein further comprises one or more syntrophin binding domains of dystrophin and/or one or more coiled coil domains of dystrophin.

4. The recombinant nucleic acid of any one of claims 1-3, wherein the spectrin-like repeat 1 is directly coupled to the spectrin-like repeat 17.

5. The recombinant nucleic acid of any one of claims 1-4, wherein the spectrin-like repeat 19 is present and is directly coupled to spectrin-like repeat 24.

6. The recombinant nucleic acid of any one of claims 1-4, wherein the spectrin-like repeat 19 is present and is directly coupled to hinge domain 4.

7. The method of any one of claims 1-4, wherein the spectrin-like repeat 17 is directly coupled to hinge domain 3 or spectrin-like repeat 22.

8. A recombinant nucleic acid comprising a nucleotide sequence encoding a micro-dystrophin protein comprising: an amino-terminal actin-binding domain, hinge domain 1, spectrin-like repeat 1, spectrin-like repeat 2, and spectrin-like repeat 17, wherein the micro-dystrophin protein does not contain spectrin-like repeat 3 of dystrophin, and wherein the micro-dystrophin protein optionally further comprises spectrin-like repeat 16.

9. The recombinant nucleic acid of claim 8, wherein the micro-dystrophin protein further comprises one or more of: hinge domain 3, spectrin-like repeat 22, spectrin-like repeat 23, spectrin-like repeat 24, hinge domain 4, and dystroglycan binding site of dystrophin.

10. The recombinant nucleic acid of claim 8 or 9, wherein the micro-dystrophin protein further comprises one or more syntrophin binding domains of dystrophin.

11. The recombinant nucleic acid of any one of claims 8-10, wherein the micro-dystrophin protein further comprises one or more coiled coil domains of dystrophin.

12. The recombinant nucleic acid of any one of claims 8-11, wherein the spectrin-like repeat 2 is directly coupled to spectrin-like repeat 16 or spectrin-like 17.

13. The recombinant nucleic acid of any one of claims 8-12, wherein the spectrin-like repeat 17 is directly coupled to spectrin-like repeat 24.

14. The recombinant nucleic acid of any one of claims 8-12, wherein the spectrin-like repeat 17 is directly coupled to hinge domain 3 or hinge domain 4.

15. The recombinant nucleic acid of any preceding claim, wherein the micro-dystrophin protein comprises the region of dystrophin from spectrin-like repeat 24 to the C-terminus.

16. The recombinant nucleic acid of any one of claims 1-14, wherein the micro-dystrophin protein comprises the region of dystrophin from spectrin-like repeat 24 to the end of the proline rich region following the first coiled coil domain.

17. The recombinant nucleic acid of any one of claims 1-14, wherein the micro-dystrophin protein comprises the region of dystrophin from hinge domain 4 to the C-terminus.

18. The recombinant nucleic acid of any one of claims 1-14, wherein the micro-dystrophin protein comprises the region of dystrophin from hinge domain 4 to the end of the proline rich region following the first coiled coil domain.

19. The recombinant nucleic acid of any one of claims 1-14, wherein the micro-dystrophin protein comprises the region of dystrophin from hinge domain 4 to the end of the second syntrophin binding domain.

20. The recombinant nucleic acid of any preceding claim, wherein the recombinant nucleic acid is less than 5 kb in length.

21. The recombinant nucleic acid of any preceding claim, wherein a promoter is operably linked to the nucleotide sequence encoding the micro-dystrophin protein.

22. The recombinant nucleic acid of claim 21, wherein the promoter is a cardiac-specific promoter.

23. The recombinant nucleic acid of claim 22, wherein the cardiac-specific promoter is a cardiac troponin T (cTnT) promoter.

24. The recombinant nucleic acid of claim 21, wherein the promoter is a skeletal muscle-specific promoter.

25. The recombinant nucleic acid of claim 24, wherein the skeletal muscle-specific promoter is a skeletal muscle alpha-actin promoter.

26. The recombinant nucleic acid of any preceding claim, wherein the skeletal-muscle specific promoter comprises a nucleotide sequence that is at least 80% identical to SEQ ID NO: 78 or SEQ ID NO: 80.

27. A recombinant nucleic acid comprising a nucleotide sequence encoding a micro-dystrophin protein, wherein the nucleotide sequence encoding the micro-dystrophin protein comprises a nucleotide sequence that is at least 80% identical to the nucleotide sequence of any one of SEQ ID Nos. 7-12, 22-39, 92-97, 109-114, and 116-118.

28. A recombinant nucleic acid comprising a nucleotide sequence encoding a micro-dystrophin protein that comprises an amino acid sequence that is at least 80% identical to the amino acid sequence of any one of SEQ ID Nos. 1-6, 13-21, 86-91, 98, 106-108, and 115.

29. A micro-dystrophin protein encoded by the recombinant nucleic acid of any one of claims 1-28.

30. A micro-dystrophin protein comprising an amino acid sequence that is at least 80% identical to the amino acid sequence of any one of SEQ ID Nos. 1-6 and 13-21, 86-91, 98, 106-108, and 115.

31. A recombinant adeno-associated virus (rAAV) vector comprising the recombinant nucleic acid of any one of claims 1-28.

32. An rAAV particle comprising the rAAV vector of claim 31 encapsidated in an AAV capsid.

33. The rAAV particle of claim 32, wherein the AAV capsid comprises a capsid protein derived from AAV1, AAV2, AAV3, AAV6, AAV7, AAV8, AAVrh.74, AAVrh.10, AAV2/6 or AAV9 serotypes.

34. A composition comprising the rAAV particle of claim 32 or 33.

35. A method of treating Duchenne muscular dystrophy (DMD) in a subject in need thereof, comprising administering to the subject the recombinant nucleic acid of any one of claims 1-28, the micro-dystrophin protein of claim 29 or 30, the rAAV particle of claim 32 or 33, or the composition of claim 34.

36. An isolated nucleic acid comprising a nucleotide sequence that is at least 80% identical to the nucleotide sequence of SEQ ID NO: 78 or SEQ ID NO: 80.

37. The isolated nucleic acid of claim 36, wherein the nucleotide sequence that is at least 80% identical to the nucleotide sequence of SEQ ID NO: 78 or SEQ ID NO: 80 is operably linked to a nucleotide sequence that is at least 80% identical to the nucleotide sequence of any one of SEQ ID Nos. 7-12, 22-39, 92-97, 109-114, and 116-118.

38. The isolated nucleic acid of claim 36, wherein the nucleotide sequence that is at least 80% identical to the nucleotide sequence of SEQ ID NO: 78 or SEQ ID NO: 80 is operably linked to a nucleotide sequence that encodes a micro-dystrophin protein that comprises an amino acid sequence that is at least 80% identical to the amino acid sequence of any one of SEQ ID Nos. 1-6, 13-21, 86-91, 98, 106-108, and 115.

39. A vector comprising the isolated nucleic acid of any one of claims 36-38.

40. The vector of claim 39, wherein the vector is a recombinant adeno-associated virus (rAAV) vector.

41. An rAAV particle comprising the rAAV vector of claim 40 encapsidated in an AAV capsid.

42. The rAAV particle of claim 41, wherein the AAV capsid comprises a capsid protein derived from AAV1, AAV2, AAV3, AAV6, AAV8, AAVrh.74, AAVrh.10, AAV2/6 or AAV9 serotypes.

43. A composition comprising the rAAV particle of claim 41 or 42.

44. A method of treating a skeletal muscle disorder in a subject in need thereof, comprising administering to the subject the isolated nucleic acid of any one of claims 36-38, the rAAV particle of claim 41 or 42, or the composition of claim 43.

45. A method of treating Duchenne muscular dystrophy (DMD) in a subject in need thereof, comprising administering to the subject the isolated nucleic acid of any one of claims 36-38, the rAAV particle of claim 41 or 42, or the composition of claim 43.

46. A method of treating Duchenne muscular dystrophy (DMD) in a subject in need thereof, the method comprising delivering to the subject a first recombinant adeno-associated virus (rAAV) particle comprising a nucleotide sequence encoding a first micro-dystrophin protein to cardiac muscle and a second rAAV particle comprising a nucleotide sequence encoding a second micro-dystrophin to skeletal muscle.

47. The method of claim 46, wherein the first rAAV particle comprises a cardiac-specific promoter operably linked to the nucleotide sequence encoding the first micro-dystrophin protein.

48. The method of claim 46, wherein the second rAAV particle comprises a skeletal muscle-specific promoter operably linked to the nucleotide sequence encoding the second micro-dystrophin protein.

49. The method of any one of claims 46-48, wherein the first rAAV particle comprises a capsid protein derived from AAV9, AAVrh.74, or AAVrh.10.

50. The method of any one of claims 46-49, wherein the second rAAV particle comprises a capsid protein derived from AAV8.

51. The method of any one of claims 46-50, wherein the first and second rAAV particles are delivered by the same method.

52. The method of any one of claims 46-50, wherein the first and second rAAV particles are delivered by different methods.

53. The method of any one of claims 46-51, wherein the first and second rAAV particles are delivered via one or more catheters.

54. The method of claim 53, comprising:
(i) introducing a catheter into the femoral artery and advancing to the heart;
(ii) delivering the first rAAV particle into the left and right coronary arteries;
(iii) retracting the catheter to the aortic arch; and
(iv) delivering the second rAAV particle to the subclavian and/or carotid arteries.

55. The method of claim 54, further comprising (v) retracting the catheter into the descending aorta and delivering the second rAAV particle to skeletal muscle via descending aortic branches.

56. The method of any one of claims 46-55, further comprising administering a vasodilator to the subject prior to the delivery of the first and second rAAV particles.

57. The method of claim 56, wherein the vasodilator is a PDE5 inhibitor.

58. The method of claim 57, wherein the PDE5 inhibitor is sildenafil or tadalafil.

59. The method of any one of claims 56-58, wherein the vasodilator is administered 10 minutes, 20 minutes, 30 minutes, 40 minutes, 50 minutes, or 1 hour prior to the delivery of first rAAV and/or second rAAV.

60. The method of any one of claims 46-59, wherein a blood pressure cuff is inflated on each limb at the time of delivery of the first rAAV particle and/or the second rAAV particle.

61. The method of any one of claims 46-59, wherein the first micro-dystrophin protein comprises: an amino-terminal actin-binding domain, hinge domain 1, spectrin-like repeat 1, and spectrin-like repeat 17, wherein the micro-dystrophin protein does not contain spectrin-like repeat 2 and/or spectrin-like repeat 3 of dystrophin, and wherein the micro-dystrophin protein optionally further comprises spectrin-like repeat 18, and/or spectrin-like repeat 19.

62. The method of claim 61, wherein the first micro-dystrophin protein further comprises one or more of: spectrin-like repeat 16, hinge domain 3, spectrin-like repeat 21, spectrin-like repeat 22, spectrin-like repeat 24, hinge domain 4, and dystroglycan binding site of dystrophin.

63. The method of claim 61 or 62, wherein the first micro-dystrophin protein further comprises one or more syntrophin binding domains of dystrophin and/or one or more coiled coil domains of dystrophin.

64. The method of any one of claims 61-63, wherein the spectrin-like repeat 1 of the first micro-dystrophin protein is directly coupled to the spectrin-like repeat 17.

65. The method of any one of claims 61-64, wherein the spectrin-like repeat 19 is present in the first micro-dystrophin protein and is directly coupled to spectrin-like repeat 24.

66. The method of any one of claims 61-64, wherein the spectrin-like repeat 19 is present in the first micro-dystrophin protein and is directly coupled to hinge domain 4.

67. The method of any one of claims 61-64, wherein the spectrin-like repeat 17 is directly coupled to hinge domain 3 or spectrin-like repeat 22.

68. The method of any one of claims 46-67, wherein the second micro-dystrophin protein comprises: an amino-terminal actin-binding domain, hinge domain 1, spectrin-like repeat 1, spectrin-like repeat 2, and spectrin-like repeat 17, wherein the micro-dystrophin protein does not contain spectrin-like repeat 3 of dystrophin and wherein the micro-dystrophin protein optionally further comprises spectrin-like repeat 16.

69. The method of claim 68, wherein the second micro-dystrophin protein further comprises one or more of: hinge domain 3, spectrin-like repeat 22, spectrin-like repeat 23, spectrin-like repeat 24, hinge domain 4, and dystroglycan binding site of dystrophin.

70. The method of claim 68 or 69, wherein the second micro-dystrophin protein further comprises one or more syntrophin binding domains of dystrophin.

71. The method of any one of claims 68-70, wherein the second micro-dystrophin protein further comprises one or more coiled coil domains of dystrophin.

72. The method of any one of claims 68-71, wherein the spectrin-like repeat 2 of the second micro-dystrophin protein is directly coupled to spectrin-like repeat 16 or spectrin-like repeat 17.

73. The method of any one of claims 68-72, wherein the spectrin-like repeat 17 of the second micro-dystrophin protein is directly coupled to spectrin-like repeat 24.

74. The method of any one of claims 68-72, wherein the spectrin-like repeat 17 of the second micro-dystrophin protein is directly coupled to hinge domain 3 or hinge domain 4.

75. The method of any one of 46-74, wherein the first or second micro-dystrophin protein comprises the region of dystrophin from spectrin-like repeat 24 to the C-terminus.

76. The method of any one of claims 46-74, wherein the first or second micro-dystrophin protein comprises the region of dystrophin from spectrin-like repeat 24 to the end of the proline rich region following the first coiled coil domain.

77. The method of any one of claims 46-74, wherein the first or second micro-dystrophin protein comprises the region of dystrophin from hinge domain 4 to the C-terminus.

78. The method of any one of claims 46-74, wherein the first or second micro-dystrophin protein comprises the region of dystrophin from hinge domain 4 to the end of the proline rich region following the first coiled coil domain.

79. The method of any one of claims 46-74, wherein the first or second micro-dystrophin protein comprises the region of dystrophin from hinge domain 4 to the end of the second syntrophin binding domain.

80. A method of delivering an rAAV particle to a skeletal muscle in a subject, the method comprising delivering the rAAV particle via a catheter.

81. The method of claim 80, comprising delivering the rAAV particle to the subclavian and/or carotid arteries.

82. The method of claim 80 or 81, wherein the catheter is first introduced into the femoral artery and advanced to the subclavian and/or carotid arteries.

83. The method of any one of claims 80-82, further comprising retracting the catheter into the descending aorta and delivering the rAAV particle to skeletal muscle via descending aortic branches.

84. The method of any one of claims 80-83, comprising administering a vasodilator.

85. The method of 84, wherein the vasodilator is a PDE5 inhibitor.

86. The method of claim 85, wherein the PDE5 inhibitor is sildenafil or tadalafil.

87. The method of any one of claims 84-86, wherein the vasodilator is administered 10 minutes, 20 minutes, 30 minutes, 40 minutes, 50 minutes, or 1 hour prior to the delivery of the rAAV particle.

88. The method of any one of claims 80-87, further comprising inflating a blood pressure cuff on each limb at the time of delivery of the rAAV particle.

89. The method of any one of claims 80-88, wherein the skeletal muscle is fast-twitch or slow-twitch.


SEQUENCE LISTING (121 sequences)

SEQ ID NO: 1 - protein, 1263 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETP

SEQ ID NO: 2 - protein, 1286 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 3 - protein, 1223 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 4 - protein, 1407 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 5 - protein, 1418 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 6 - protein, 1539 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 7 - DNA, 3789 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactccc

SEQ ID NO: 8 - DNA, 3858 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 9 - DNA, 3669 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 10 - DNA, 4221 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 11 - DNA, 4254 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 12 - DNA, 4617 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 13 - protein, 1277 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETP

SEQ ID NO: 14 - protein, 1237 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 15 - protein, 1300 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 16 - protein, 1421 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 17 - protein, 1431 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 18 - protein, 1432 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 19 - protein, 1448 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 20 - protein, 1553 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 21 - protein, 1358 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 22 - protein, 3831 aa, Artificial Sequence
  Synthetic
ATGCTTTGGTGGGAAGAAGTAGAGGACTGTTATGAAAGAGAAGATGTTCAAAAGAAAACATTCACAAAATGGGTAAATGCACAATTTTCTAAGTTTGGGAAGCAGCATATTGAGAACCTCTTCAGTGACCTACAGGATGGGAGGCGCCTCCTAGACCTCCTCGAAGGCCTGACAGGGCAAAAACTGCCAAAAGAAAAAGGATCCACAAGAGTTCATGCCCTGAACAATGTCAACAAGGCACTGCGGGTTTTGCAGAACAATAATGTTGATTTAGTGAATATTGGAAGTACTGACATCGTAGATGGAAATCATAAACTGACTCTTGGTTTGATTTGGAATATAATCCTCCACTGGCAGGTCAAAAATGTAATGAAAAATATCATGGCTGGATTGCAACAAACCAACAGTGAAAAGATTCTCCTGAGCTGGGTCCGACAATCAACTCGTAATTATCCACAGGTTAATGTAATCAACTTCACCACCAGCTGGTCTGATGGCCTGGCTTTGAATGCTCTCATCCATAGTCATAGGCCAGACCTATTTGACTGGAATAGTGTGGTTTGCCAGCAGTCAGCCACACAACGACTGGAACATGCATTCAACATCGCCAGATATCAATTAGGCATAGAGAAACTACTCGATCCTGAAGATGTTGATACCACCTATCCAGATAAGAAGTCCATCTTAATGTACATCACATCACTCTTCCAAGTTTTGCCTCAACAAGTGAGCATTGAAGCCATCCAGGAAGTGGAAATGTTGCCAAGGCCACCTAAAGTGACTAAAGAAGAACATTTTCAGTTACATCATCAAATGCACTATTCTCAACAGATCACGGTCAGTCTAGCACAGGGATATGAGAGAACTTCTTCCCCTAAGCCTCGATTCAAGAGCTATGCCTACACACAGGCTGCTTATGTCACCACCTCTGACCCTACACGGAGCCCATTTCCTTCACAGCATTTGGAAGCTCCTGAAGACAAGTCATTTGGCAGTTCATTGATGGAGAGTGAAGTAAACCTGGACCGTTATCAAACAGCTTTAGAAGAAGTATTATCGTGGCTTCTTTCTGCTGAGGACACATTGCAAGCACAAGGAGAGATTTCTAATGATGTGGAAGTGGTGAAAGACCAGTTTCATACTCATGAGGGGTACATGATGGATTTGACAGCCCATCAGGGCCGGGTTGGTAATATTCTACAATTGGGAAGTAAGCTGATTGGAACAGGAAAATTATCAGAAGATGAAGAAACTGAAGTACAAGAGCAGATGAATCTCCTAAATTCAAGATGGGAATGCCTCAGGGTAGCTAGCATGGAAAAACAAAGCAATTTACATAGAGTTTTAATGGATCTCCAGAATCAGAAACTGAAAGAGTTGAATGACTGGCTAACAAAAACAGAAGAAAGAACAAGGAAAATGGAGGAAGAGCCTCTTGGACCTGATCTTGAAGACCTAAAACGCCAAGTACAACAACATAAGGTGCTTCAAGAAGATCTAGAACAAGAACAAGTCAGGGTCAATTCTCTCACTCACATGGTGGTGGTAGTTGATGAATCTAGTGGAGATCACGCAACTGCTGCTTTGGAAGAACAACTTAAGGTATTGGGAGATCGATGGGCAAACATCTGTAGATGGACAGAAGACCGCTGGGTTCTTTTACAAGACCACACTGTCCGTGAAGAAACGATGATGGTGATGACTGAAGACATGCCTTTGGAAATTTCTTATGTGCCTTCTACTTATTTGACTGAAATCACTCATGTCTCACAAGCCCTATTAGAAGTGGAACAACTTCTCAATGCTCCTGACCTCTGTGCTAAGGACTTTGAAGATCTCTTTAAGCAAGAGGAGTCTCTGAAGAATATAAAAGATAGTCTACAACAAAGCTCAGGTCGGATTGACATTATTCATAGCAAGAAGACAGCAGCATTGCAAAGTGCAACGCCTGTGGAAAGGGTGAAGCTACAGGAAGCTCTCTCCCAGCTTGATTTCCAATGGGAAAAAGTTAACAAAATGTACAAGGACCGACAAGGGCGATTTGACAGATCTGTTGAGAAATGGCGGCGTTTTCATTATGATATAAAGATATTTAATCAGTGGCTAACAGAAGCTGAACAGTTTCTCAGAAAGACACAAATTCCTGAGAATTGGGAACATGCTAAATACAAATGGTATCTTAAGGAACTCCAGGATGGCATTGGGCAGCGGCAAACTGTTGTCAGAACATTGAATGCAACTGGGGAAGAAATAATTCAGCAATCCTCAAAAACAGATGCCAGTATTCTACAGGAAAAATTGGGAAGCCTGAATCTGCGGTGGCAGGAGGTCTGCAAACAGCTGTCAGACAGAAAAAAGAGGCTAGAAGAACAAAAGTGGCAGAGAAAAATAGATGAGACCCTTGAAAGACTCCGGGAACTTCAAGAGGCCACGGATGAGCTGGACCTCAAGCTGCGCCAAGCTGAGGTGATCAAGGGATCCTGGCAGCCCGTGGGCGATCTCCTCATTGACTCTCTCCAAGATCACCTCGAGAAAGTCAAGGCACTTCGAGGAGAAATTGCGCCTCTGAAAGAGAACGTGAGCCACGTCAATGACCTTGCTCGCCAGCTTACCACTTTGGGCATTCAGCTCTCACCGTATAACCTCAGCACTCTGGAAGACCTGAACACCAGATGGAAGCTTCTGCAGGTGGCCGTCGAGGACCGAGTCAGGCAGCTGCATGAAGCCCACAGGGACTTTGGTCCAGCATCTCAGCACTTTCTTTCCACGTCTGTCCAGGGTCCCTGGGAGAGAGCCATCTCGCCAAACAAAGTGCCCTACTATATCAACCACGAGACTCAAACAACTTGCTGGGACCATCCCAAAATGACAGAGCTCTACCAGTCTTTAGCTGACCTGAATAATGTCAGATTCTCAGCTTATAGGACTGCCATGAAACTCCGAAGACTGCAGAAGGCCCTTTGCTTGGATCTCTTGAGCCTGTCAGCTGCATGTGATGCCTTGGACCAGCACAACCTCAAGCAAAATGACCAGCCCATGGATATCCTGCAGATTATTAATTGTTTGACCACTATTTATGACCGCCTGGAGCAAGAGCACAACAATTTGGTCAACGTCCCTCTCTGCGTGGATATGTGTCTGAACTGGCTGCTGAATGTTTATGATACGGGACGAACAGGGAGGATCCGTGTCCTGTCTTTTAAAACTGGCATCATTTCCCTGTGTAAAGCACATTTGGAAGACAAGTACAGATACCTTTTCAAGCAAGTGGCAAGTTCAACAGGATTTTGTGACCAGCGCAGGCTGGGCCTCCTTCTGCATGATTCTATCCAAATTCCAAGACAGTTGGGTGAAGTTGCATCCTTTGGGGGCAGTAACATTGAGCCAAGTGTCCGGAGCTGCTTCCAATTTGCTAATAATAAGCCAGAGATCGAAGCGGCCCTCTTCCTAGACTGGATGAGACTGGAACCCCAGTCCATGGTGTGGCTGCCCGTCCTGCACAGAGTGGCTGCTGCAGAAACTGCCAAGCATCAGGCCAAATGTAACATCTGCAAAGAGTGTCCAATCATTGGATTCAGGTACAGGAGTCTAAAGCACTTTAATTATGACATCTGCCAAAGCTGCTTTTTTTCTGGTCGAGTTGCAAAAGGCCATAAAATGCACTATCCCATGGTGGAATATTGCACTCCGACTACATCAGGAGAAGATGTTCGAGACTTTGCCAAGGTACTAAAAAACAAATTTCGAACCAAAAGGTATTTTGCGAAGCATCCCCGAATGGGCTACCTGCCAGTGCAGACTGTCTTAGAGGGGGACAACATGGAAACTCCC

SEQ ID NO: 23 - DNA, 3711 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 24 - DNA, 3900 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 25 - DNA, 4263 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 26 - DNA, 4293 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 27 - DNA, 4296 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 28 - DNA, 4344 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 29 - DNA, 4659 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 30 - DNA, 4074 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 31 - DNA, 3831 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagtggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagaccccc

SEQ ID NO: 32 - DNA, 3711 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 33 - DNA, 3900 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccc

SEQ ID NO: 34 - DNA, 4263 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagtggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccc

SEQ ID NO: 35 - DNA, 4293 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacctgagcctgcaggagctgctggtgtggctgcagctgaaggacgacgagctgagccggcaggcccccatcggcggcgacttccccgccgtgcagaagcagaacgacgtgcaccgggccttcaagcgggagctgaagaccaaggagcccgtgatcatgagcaccctggagaccgtgcggatcttcctgaccgagcagcccctggagggcctggagaagctgtaccaggagccccgggagctgccccccgaggagcgggcccagaacgtgacccggctgctgcggaagcaggccgaggaggtgaacaccgagtgggagaagctgaacctgcacagcgccgactggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccccgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 36 - DNA, 4296 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccccgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 37 - DNA, 4344 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagtggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccccgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 38 - DNA, 4659 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagtggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccccgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 39 - DNA, 4074 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagtggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 40 - protein, 325 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAP

SEQ ID NO: 41 - protein, 126 aa, Homo sapiens
EDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMD

SEQ ID NO: 42 - protein, 105 aa, Homo sapiens
LQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQD

SEQ ID NO: 43 - protein, 118 aa, Homo sapiens
ILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQP

SEQ ID NO: 44 - protein, 123 aa, Artificial Sequence
  Synthetic
HTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQG

SEQ ID NO: 45 - protein, 113 aa, Homo sapiens
RFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQK

SEQ ID NO: 46 - protein, 327 aa, Artificial Sequence
  Synthetic
RFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQ

SEQ ID NO: 47 - protein, 114 aa, Artificial Sequence
  Synthetic
HLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSAD

SEQ ID NO: 48 - protein, 121 aa, Homo sapiens
WQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDF

SEQ ID NO: 49 - protein, 364 aa, Homo sapiens
GPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETP

SEQ ID NO: 50 - protein, 81 aa, Homo sapiens
VTLINFWPVDSAPASSPQLSHDDTHSRIEHYASRLAEMENSNGSYLNDSISPNESIDDEHLLIQHYCQSLNQDSPLSQPRS

SEQ ID NO: 51 - protein, 63 aa, Homo sapiens
PAQILISLESEERGELERILADLEEENRNLQAEYDRLKQQHEHKGLSPLPSPPEMMPTSPQSP

SEQ ID NO: 52 - protein, 132 aa, Homo sapiens
RDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 53 - DNA, 975 bp, Homo sapiens
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcct

SEQ ID NO: 54 - DNA, 975 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggccccc

SEQ ID NO: 55 - DNA, 378 bp, Homo sapiens
gaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggat

SEQ ID NO: 56 - DNA, 378 bp, Artificial Sequence
  Synthetic
gaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggac

SEQ ID NO: 57 - DNA, 315 bp, Homo sapiens
ctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagac

SEQ ID NO: 58 - DNA, 315 bp, Artificial Sequence
  Synthetic
ctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggac

SEQ ID NO: 59 - DNA, 369 bp, Artificial Sequence
  Synthetic
cacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaaggg

SEQ ID NO: 60 - DNA, 369 bp, Artificial Sequence
  Synthetic
cacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggc

SEQ ID NO: 61 - DNA, 339 bp, Homo sapiens
cgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaag

SEQ ID NO: 62 - DNA, 339 bp, Artificial Sequence
  Synthetic
cggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaag

SEQ ID NO: 63 - DNA, 981 bp, Homo sapiens
cgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcag

SEQ ID NO: 64 - DNA, 342 bp, Artificial Sequence
  Synthetic
cacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgac

SEQ ID NO: 65 - DNA, 342 bp, Artificial Sequence
  Synthetic
cacctgagcctgcaggagctgctggtgtggctgcagctgaaggacgacgagctgagccggcaggcccccatcggcggcgacttccccgccgtgcagaagcagaacgacgtgcaccgggccttcaagcgggagctgaagaccaaggagcccgtgatcatgagcaccctggagaccgtgcggatcttcctgaccgagcagcccctggagggcctggagaagctgtaccaggagccccgggagctgccccccgaggagcgggcccagaacgtgacccggctgctgcggaagcaggccgaggaggtgaacaccgagtgggagaagctgaacctgcacagcgccgac

SEQ ID NO: 66 - DNA, 363 bp, Homo sapiens
tggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggacttt

SEQ ID NO: 67 - DNA, 363 bp, Artificial Sequence
  Synthetic
tggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttc

SEQ ID NO: 68 - DNA, 1092 bp, Homo sapiens
ggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactccc

SEQ ID NO: 69 - DNA, 1092 bp, Artificial Sequence
  Synthetic
ggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagaccccc

SEQ ID NO: 70 - DNA, 243 bp, Homo sapiens
gttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 71 - DNA, 243 bp, Artificial Sequence
  Synthetic
gtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 72 - DNA, 189 bp, Homo sapiens
cctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 73 - DNA, 189 bp, Artificial Sequence
  Synthetic
cccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccc

SEQ ID NO: 74 - DNA, 396 bp, Homo sapiens
cgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 75 - DNA, 396 bp, Artificial Sequence
  Synthetic
cgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 76 - DNA, 106 bp, Artificial Sequence
  Synthetic
tctagaaaatctgaaaggcatagccccatatatcagtgatataaatagaacctgcagcaggctctggtaaatgatgactacaaggtggactgggaggcagcccggc

SEQ ID NO: 77 - DNA, 278 bp, Artificial Sequence
  Synthetic
aggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaccatgg

SEQ ID NO: 78 - DNA, 380 bp, Artificial Sequence
  Synthetic
tctagaaaatctgaaaggcatagccccatatatcagtgatataaatagaacctgcagcaggctctggtaaatgatgactacaaggtggactgggaggcagcccggcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccacc

SEQ ID NO: 79 - DNA, 205 bp, Artificial Sequence
  Synthetic
tctagaaaatctgaaaggcatagccccatatatcagtgatataaatagaacctgcagcaggctctggtaaatgatgactacaaggtggactgggaggcagcccggcaatctgaaaggcatagccccatatatcagtgatataaatagaacctgcagcaggctctggtaaatgatgactacaaggtggactgggaggcagcccggc

SEQ ID NO: 80 - DNA, 479 bp, Artificial Sequence
  Synthetic
tctagaaaatctgaaaggcatagccccatatatcagtgatataaatagaacctgcagcaggctctggtaaatgatgactacaaggtggactgggaggcagcccggcaatctgaaaggcatagccccatatatcagtgatataaatagaacctgcagcaggctctggtaaatgatgactacaaggtggactgggaggcagcccggcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccacc

SEQ ID NO: 81 - protein, 324 aa, Homo sapiens
RSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQ

SEQ ID NO: 82 - protein, 123 aa, Artificial Sequence
  Synthetic
ASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSAD

SEQ ID NO: 83 - DNA, 972 bp, Homo sapiens
agatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcag

SEQ ID NO: 84 - DNA, 369 bp, Artificial Sequence
  Synthetic
gccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgac

SEQ ID NO: 85 - DNA, 369 bp, Artificial Sequence
  Synthetic
gccagcagcgaccagtggaagcggctgcacctgagcctgcaggagctgctggtgtggctgcagctgaaggacgacgagctgagccggcaggcccccatcggcggcgacttccccgccgtgcagaagcagaacgacgtgcaccgggccttcaagcgggagctgaagaccaaggagcccgtgatcatgagcaccctggagaccgtgcggatcttcctgaccgagcagcccctggagggcctggagaagctgtaccaggagccccgggagctgccccccgaggagcgggcccagaacgtgacccggctgctgcggaagcaggccgaggaggtgaacaccgagtgggagaagctgaacctgcacagcgccgac

SEQ ID NO: 86 - protein, 1260 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETP

SEQ ID NO: 87 - protein, 1283 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 88 - protein, 1220 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 89 - protein, 1404 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSP

SEQ ID NO: 90 - protein, 1415 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 91 - protein, 1536 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 92 - DNA, 3780 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactccc

SEQ ID NO: 93 - DNA, 3849 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 94 - DNA, 3660 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 95 - DNA, 4212 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccc

SEQ ID NO: 96 - DNA, 4245 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 97 - DNA, 4608 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagtggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 98 - protein, 1440 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 99 - DNA, 4320 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacgccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatg

SEQ ID NO: 100 - DNA, 141 bp, Homo sapiens
cagcctgacctagctcctggactgaccactattggagcctctcctactcagactgttactctggtgacacaacctgtggttactaaggaaactgccatctccaaactagaaatgccatcttccttgatgttggaggtacct

SEQ ID NO: 101 - DNA, 141 bp, Artificial Sequence
  Synthetic
cagcccgacctggcccccggcctgaccaccatcggcgccagccccacccagaccgtgaccctggtgacccagcccgtggtgaccaaggagaccgccatcagcaagctggagatgcccagcagcctgatgctggaggtgccc

SEQ ID NO: 102 - DNA, 315 bp, Homo sapiens
aaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttg

SEQ ID NO: 103 - DNA, 315 bp, Artificial Sequence
  Synthetic
aaggacagcacccagtggctggaggccaaggaggaggccgagcaggtgctgggccaggcccgggccaagctggagagctggaaggagggcccctacaccgtggacgccatccagaagaagatcaccgagaccaagcagctggccaaggacctgcggcagtggcagaccaacgtggacgtggccaacgacctggccctgaagctgctgcgggactacagcgccgacgacacccggaaggtgcacatgatcaccgagaacatcaacgccagctggcggagcatccacaagcgggtgagcgagcgggaggccgccctg

SEQ ID NO: 104 - DNA, 378 bp, Artificial Sequence
  Synthetic
gaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctg

SEQ ID NO: 105 - DNA, 378 bp, Artificial Sequence
  Synthetic
gaggagacccaccggctgctgcagcagttccccctggacctggagaagttcctggcctggctgaccgaggccgagaccaccgccaacgtgctgcaggacgccacccggaaggagcggctgctggaggacagcaagggcgtgaaggagctgatgaagcagtggcaggacctgcagggcgagatcgaggcccacaccgacgtgtaccacaacctggacgagaacagccagaagatcctgcggagcctggagggcagcgacgacgccgtgctgctgcagcggcggctggacaacatgaacttcaagtggagcgagctgcggaagaagagcctgaacatccggagccacctggaggccagcagcgaccagtggaagcggctg

SEQ ID NO: 106 - protein, 1287 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKQPDLAPGLTTIGASPTQTVTLVTQPVVTKETAISKLEMPSSLMLEVPKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 107 - protein, 1182 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLS

SEQ ID NO: 108 - protein, 1363 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 109 - DNA, 3861 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagcagcctgacctagctcctggactgaccactattggagcctctcctactcagactgttactctggtgacacaacctgtggttactaaggaaactgccatctccaaactagaaatgccatcttccttgatgttggaggtacctaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 110 - DNA, 3861 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggaccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagcagcccgacctggcccccggcctgaccaccatcggcgccagccccacccagaccgtgaccctggtgacccagcccgtggtgaccaaggagaccgccatcagcaagctggagatgcccagcagcctgatgctggaggtgcccaaggacagcacccagtggctggaggccaaggaggaggccgagcaggtgctgggccaggcccgggccaagctggagagctggaaggagggcccctacaccgtggacgccatccagaagaagatcaccgagaccaagcagctggccaaggacctgcggcagtggcagaccaacgtggacgtggccaacgacctggccctgaagctgctgcgggactacagcgccgacgacacccggaaggtgcacatgatcaccgagaacatcaacgccagctggcggagcatccacaagcgggtgagcgagcgggaggccgccctggaggagacccaccggctgctgcagcagttccccctggacctggagaagttcctggcctggctgaccgaggccgagaccaccgccaacgtgctgcaggacgccacccggaaggagcggctgctggaggacagcaagggcgtgaaggagctgatgaagcagtggcaggacctgcagggcgagatcgaggcccacaccgacgtgtaccacaacctggacgagaacagccagaagatcctgcggagcctggagggcagcgacgacgccgtgctgctgcagcggcggctggacaacatgaacttcaagtggagcgagctgcggaagaagagcctgaacatccggagccacctggaggccagcagcgaccagtggaagcggctgggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 111 - DNA, 3546 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaaggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtcc

SEQ ID NO: 112 - DNA, 3546 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggaccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaaggaggagacccaccggctgctgcagcagttccccctggacctggagaagttcctggcctggctgaccgaggccgagaccaccgccaacgtgctgcaggacgccacccggaaggagcggctgctggaggacagcaagggcgtgaaggagctgatgaagcagtggcaggacctgcagggcgagatcgaggcccacaccgacgtgtaccacaacctggacgagaacagccagaagatcctgcggagcctggagggcagcgacgacgccgtgctgctgcagcggcggctggacaacatgaacttcaagtggagcgagctgcggaagaagagcctgaacatccggagccacctggaggccagcagcgaccagtggaagcggctgggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagc

SEQ ID NO: 113 - DNA, 4089 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 114 - DNA, 4089 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggaccacaccgtgcgggaggagaccatgatggtgatgaccgaggacatgcccctggagatcagctacgtgcccagcacctacctgaccgagatcacccacgtgagccaggccctgctggaggtggagcagctgctgaacgcccccgacctgtgcgccaaggacttcgaggacctgttcaagcaggaggagagcctgaagaacatcaaggacagcctgcagcagagcagcggccggatcgacatcatccacagcaagaagaccgccgccctgcagagcgccacccccgtggagcgggtgaagctgcaggaggccctgagccagctggacttccagtgggagaaggtgaacaagatgtacaaggaccggcagggccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagaaggacagcacccagtggctggaggccaaggaggaggccgagcaggtgctgggccaggcccgggccaagctggagagctggaaggagggcccctacaccgtggacgccatccagaagaagatcaccgagaccaagcagctggccaaggacctgcggcagtggcagaccaacgtggacgtggccaacgacctggccctgaagctgctgcgggactacagcgccgacgacacccggaaggtgcacatgatcaccgagaacatcaacgccagctggcggagcatccacaagcgggtgagcgagcgggaggccgccctggaggagacccaccggctgctgcagcagttccccctggacctggagaagttcctggcctggctgaccgaggccgagaccaccgccaacgtgctgcaggacgccacccggaaggagcggctgctggaggacagcaagggcgtgaaggagctgatgaagcagtggcaggacctgcagggcgagatcgaggcccacaccgacgtgtaccacaacctggacgagaacagccagaagatcctgcggagcctggagggcagcgacgacgccgtgctgctgcagcggcggctggacaacatgaacttcaagtggagcgagctgcggaagaagagcctgaacatccggagccacctggaggccagcagcgaccagtggaagcggctgggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 115 - protein, 1287 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKQPDLAPGLTTIGASPTQTVTLVTQPVVTKETAISKLEMPSSLMLEVPEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRS

SEQ ID NO: 116 - DNA, 3861 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagcagcctgacctagctcctggactgaccactattggagcctctcctactcagactgttactctggtgacacaacctgtggttactaaggaaactgccatctccaaactagaaatgccatcttccttgatgttggaggtacctgaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagt

SEQ ID NO: 117 - DNA, 3861 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggaccggttcgaccggagcgtggagaagtggcggcggttccactacgacatcaagatcttcaaccagtggctgaccgaggccgagcagttcctgcggaagacccagatccccgagaactgggagcacgccaagtacaagtggtacctgaaggagctgcaggacggcatcggccagcggcagaccgtggtgcggaccctgaacgccaccggcgaggagatcatccagcagagcagcaagaccgacgccagcatcctgcaggagaagctgggcagcctgaacctgcggtggcaggaggtgtgcaagcagctgagcgaccggaagaagcggctggaggagcagaagcagcccgacctggcccccggcctgaccaccatcggcgccagccccacccagaccgtgaccctggtgacccagcccgtggtgaccaaggagaccgccatcagcaagctggagatgcccagcagcctgatgctggaggtgcccgaggagacccaccggctgctgcagcagttccccctggacctggagaagttcctggcctggctgaccgaggccgagaccaccgccaacgtgctgcaggacgccacccggaaggagcggctgctggaggacagcaagggcgtgaaggagctgatgaagcagtggcaggacctgcagggcgagatcgaggcccacaccgacgtgtaccacaacctggacgagaacagccagaagatcctgcggagcctggagggcagcgacgacgccgtgctgctgcagcggcggctggacaacatgaacttcaagtggagcgagctgcggaagaagagcctgaacatccggagccacctggaggccagcagcgaccagtggaagcggctgggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagc

SEQ ID NO: 118 - DNA, 4320 bp, Artificial Sequence
  Synthetic
atgctgtggtgggaggaggtggaggactgctacgagcgggaggacgtgcagaagaagaccttcaccaagtgggtgaacgcccagttcagcaagttcggcaagcagcacatcgagaacctgttcagcgacctgcaggacggccggcggctgctggacctgctggagggcctgaccggccagaagctgcccaaggagaagggcagcacccgggtgcacgccctgaacaacgtgaacaaggccctgcgggtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgcggcagagcacccggaactacccccaggtgaacgtgatcaacttcaccaccagctggagcgacggcctggccctgaacgccctgatccacagccaccggcccgacctgttcgactggaacagcgtggtgtgccagcagagcgccacccagcggctggagcacgccttcaacatcgcccggtaccagctgggcatcgagaagctgctggaccccgaggacgtggacaccacctaccccgacaagaagagcatcctgatgtacatcaccagcctgttccaggtgctgccccagcaggtgagcatcgaggccatccaggaggtggagatgctgccccggccccccaaggtgaccaaggaggagcacttccagctgcaccaccagatgcactacagccagcagatcaccgtgagcctggcccagggctacgagcggaccagcagccccaagccccggttcaagagctacgcctacacccaggccgcctacgtgaccaccagcgaccccacccggagccccttccccagccagcacctggaggcccccgaggacaagagcttcggcagcagcctgatggagagcgaggtgaacctggaccggtaccagaccgccctggaggaggtgctgagctggctgctgagcgccgaggacaccctgcaggcccagggcgagatcagcaacgacgtggaggtggtgaaggaccagttccacacccacgagggctacatgatggacctgaccgcccaccagggccgggtgggcaacatcctgcagctgggcagcaagctgatcggcaccggcaagctgagcgaggacgaggagaccgaggtgcaggagcagatgaacctgctgaacagccggtgggagtgcctgcgggtggccagcatggagaagcagagcaacctgcaccgggtgctgatggacctgcagaaccagaagctgaaggagctgaacgactggctgaccaagaccgaggagcggacccggaagatggaggaggagcccctgggccccgacctggaggacctgaagcggcaggtgcagcagcacaaggtgctgcaggaggacctggagcaggagcaggtgcgggtgaacagcctgacccacatggtggtggtggtggacgagagcagcggcgaccacgccaccgccgccctggaggagcagctgaaggtgctgggcgaccggtgggccaacatctgccggtggaccgaggaccggtgggtgctgctgcaggacgccagcagcgaccagtggaagcggctgcacctgagcctgcaggagctgctggtgtggctgcagctgaaggacgacgagctgagccggcaggcccccatcggcggcgacttccccgccgtgcagaagcagaacgacgtgcaccgggccttcaagcgggagctgaagaccaaggagcccgtgatcatgagcaccctggagaccgtgcggatcttcctgaccgagcagcccctggagggcctggagaagctgtaccaggagccccgggagctgccccccgaggagcgggcccagaacgtgacccggctgctgcggaagcaggccgaggaggtgaacaccgagtgggagaagctgaacctgcacagcgccgactggcagcggaagatcgacgagaccctggagcggctgcaggagctgcaggaggccaccgacgagctggacctgaagctgcggcaggccgaggtgatcaagggcagctggcagcccgtgggcgacctgctgatcgacagcctgcaggaccacctggagaaggtgaaggccctgcggggcgagatcgcccccctgaaggagaacgtgagccacgtgaacgacctggcccggcagctgaccaccctgggcatccagctgagcccctacaacctgagcaccctggaggacctgaacacccggtggaagctgctgcaggtggccgtggaggaccgggtgcggcagctgcacgaggcccaccgggacttcggccccgccagccagcacttcctgagcaccagcgtgcagggcccctgggagcgggccatcagccccaacaaggtgccctactacatcaaccacgagacccagaccacctgctgggaccaccccaagatgaccgagctgtaccagagcctggccgacctgaacaacgtgcggttcagcgcctaccggaccgccatgaagctgcggcggctgcagaaggccctgtgcctggacctgctgagcctgagcgccgcctgcgacgccctggaccagcacaacctgaagcagaacgaccagcccatggacatcctgcagatcatcaactgcctgaccaccatctacgaccggctggagcaggagcacaacaacctggtgaacgtgcccctgtgcgtggacatgtgcctgaactggctgctgaacgtgtacgacaccggccggaccggccggatccgggtgctgagcttcaagaccggcatcatcagcctgtgcaaggcccacctggaggacaagtaccggtacctgttcaagcaggtggccagcagcaccggcttctgcgaccagcggcggctgggcctgctgctgcacgacagcatccagatcccccggcagctgggcgaggtggccagcttcggcggcagcaacatcgagcccagcgtgcggagctgcttccagttcgccaacaacaagcccgagatcgaggccgccctgttcctggactggatgcggctggagccccagagcatggtgtggctgcccgtgctgcaccgggtggccgccgccgagaccgccaagcaccaggccaagtgcaacatctgcaaggagtgccccatcatcggcttccggtaccggagcctgaagcacttcaactacgacatctgccagagctgcttcttcagcggccgggtggccaagggccacaagatgcactaccccatggtggagtactgcacccccaccaccagcggcgaggacgtgcgggacttcgccaaggtgctgaagaacaagttccggaccaagcggtacttcgccaagcacccccggatgggctacctgcccgtgcagaccgtgctggagggcgacaacatggagacccccgtgaccctgatcaacttctggcccgtggacagcgcccccgccagcagcccccagctgagccacgacgacacccacagccggatcgagcactacgccagccggctggccgagatggagaacagcaacggcagctacctgaacgacagcatcagccccaacgagagcatcgacgacgagcacctgctgatccagcactactgccagagcctgaaccaggacagccccctgagccagccccggagccccgcccagatcctgatcagcctggagagcgaggagcggggcgagctggagcggatcctggccgacctggaggaggagaaccggaacctgcaggccgagtacgaccggctgaagcagcagcacgagcacaagggcctgagccccctgcccagcccccccgagatgatgcccaccagcccccagagcccccgggacgccgagctgatcgccgaggccaagctgctgcggcagcacaagggccggctggaggcccggatgcagatcctggaggaccacaacaagcagctggagagccagctgcaccggctgcggcagctgctggagcagccccaggccgaggccaaggtgaacggcaccaccgtgagcagccccagcaccagcctgcagcggagcgacagcagccagcccatgctgctgcgggtggtgggcagccagaccagcgacagcatgggcgaggaggacctgctgagccccccccaggacaccagcaccggcctggaggaggtgatggagcagctgaacaacagcttccccagcagccggggccggaacacccccggcaagcccatgcgggaggacaccatg

SEQ ID NO: 119 - protein, 47 aa, Homo sapiens
QPDLAPGLTTIGASPTQTVTLVTQPVVTKETAISKLEMPSSLMLEVP

SEQ ID NO: 120 - protein, 105 aa, Homo sapiens
KDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAAL

SEQ ID NO: 121 - protein, 126 aa, Artificial Sequence
  Synthetic
EETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRL